SEQUENCE LISTING (217 sequences)

SEQ ID NO: 1 - DNA, 6525 bp, Homo sapiens
  unsure 4449: n=unknown, may be a or g or c or t
aaaatgttgatattttctcttagcaggctgtcaaccaggttaggttcaggtcataagtttctacccacattctttgaactgtagttgtcattttagtttatttttcaaaaacttttgcagtacctttttggtctgtcttgtgtgtgccttgcagtgaacagtctggatttggacagtggtctgtctgttagttcagtttctcaagcctttgtcacactaataggattggatttatgtatgtccagcttgggaattattacaggaattaaaaacaactttttagagtgctttcctgagctctctttctatttgttcccccttctactttttgcttccctgtggctgctgtttctatcctccagccagagagctagtgtttattttctccattgtgttacacacttgtgcagctgcaaccaccatatccagggcccaatggtaggaggtagagaagaaaagcaaaagggattggcctcatcctcttacaacgatagttccattgaatagagagaaaggttttcctgcctcagagtgttggctgcactaggcttttgttactgtagtctggccctgttaccatgggattgcttgcatgtggggatacaggagaattcagaaaagaaaaaaagatttgctatttctacattctccctgagcattaagacctcccttgcccattcctcaattcaaagctaaggcttcttctggagctgcctctgtgggcggttcgggagataccaaaggagaaaaagtaccactgttgatatggtggtatttcaaattctggtctaccctatttcacatgccttgtttacttttcagagctgacagattgctgctccatgcattctgtccagtttcctaagagagacagcttggagtatgcttaatccatcttacctgggactgaaacagctgcttattttgccgttaaaaattacatgcagtttactgcgtggctccgggtttgtttgtttgtttttcctctttaataggtttattcagaaaacatgtccactgcaattagggaggtaggagtttggagacagaccagaacacttctactgaagaattacttaattaaatgcagaaccaaaaagagtagtgttcaggaaattctttttccactattttttttattttggttaatattaattagcatgatgcatccaaataagaaatatgaagaagtgcctaatatagaactcaatcctatggacaagtttactctttctaatctaattcttggatatactccagtgactaatattacaagcagcatcatgcagaaagtgtctactgatcatctacctgatgtcataattactgaagaatatacaaatgaaaaagaaatgttaacatccagtctctctaagccgagcaactttgtaggtgtggttttcaaagactccatgtcctatgaacttcgtttttttcctgatatgattccagtatcttctatttatatggattcaagagctggctgttcaaaatcatgtgaggctgctcagtactggtcctcaggtttcacagttttacaagcatccatagatgctgccattatacagttgaagaccaatgtttctctttggaaggagctggagtcaactaaagctgttattatgggagaaactgctgttgtagaaatagatacctttccccgaggagtaattttaatatacctagttatagcattttcaccttttggatactttttggcaattcatatcgtagcagaaaaagaaaaaaaaataaaagaatttttaaagataatgggacttcatgatactgccttttggctttcctgggttcttctatatacaagtttaatttttcttatgtcccttcttatggcagtcattgcgacagcttctttgttatttcctcaaagtagcagcattgtgatatttctgctttttttcctttatggattatcatctgtattttttgctttaatgctgacacctctttttaaaaaatcaaaacatgtgggaatagttgaattttttgttactgtggcttttggatttattggccttatgataatcctcatagaaagttttcccaaatcgttagtgtggcttttcagtcctttctgtcactgtacttttgtgattggtattgcacaggtcatgcatttagaagattttaatgaaggtgcttcattttcaaatttgactgcaggcccatatcctctaattattacaattatcatgctcacacttaatagtatattctatgtcctcttggctgtctatcttgatcaagtcattccaggggaatttggcttacggagatcatctttatattttctgaagccttcatattggtcaaagagtaaaagaaattatgaggagttatcagagggcaatgttaatggaaatattagttttagtgaaattattgagccagtttcttcagaatttgtaggaaaagaagccataagaattagtggtattcagaagacatacagaaagaagggtgaaaatgtggaggctttgagaaatttgtcatttgacatatatgagggtcagattactgccttacttggccacagtggaacaggaaagagtacattgatgaatattctttgtggactctgcccaccttctgatgggtttgcatctatatatggacacagagtctcagaaatagatgaaatgtttgaagcaagaaaaatgattggcatttgtccacagttagatatacactttgatgttttgacagtagaagaaaatttatcaattttggcttcaatcaaagggataccagccaacaatataatacaagaagtgcagaaggttttactagatttagacatgcagactatcaaagataaccaagctaaaaaattaagtggtggtcaaaaaagaaagctgtcattaggaattgctgttcttgggaacccaaagatactgctgctagatgaaccaacagctggaatggacccctgttctcgacatattgtatggaatcttttaaaatacagaaaagccaatcgggtgacagtgttcagtactcatttcatggatgaagctgacattcttgcagataggaaagctgtgatatcacaaggaatgctgaaatgtgttggttcttcaatgttcctcaaaagtaaatgggggatcggctaccgcctgagcatgtacatagacaaatattgtgccacagaatctctttcttcactggttaaacaacatatacctggagctactttattacaacagaatgaccaacaacttgtgtatagcttgcctttcaaggacatggacaaattttcaggtttgttttctgccctagacagtcattcaaatttgggtgtcatttcttatggtgtttccatgacgactttggaagacgtatttttaaagctagaagttgaagcagaaattgaccaagcagattatagtgtatttactcagcagccactggaggaagaaatggattcaaaatcttttgatgaaatggaacagagcttacttattctttctgaaaccaaggcttctctagtgagcaccatgagcctttggaaacaacagatgtatacaatagcaaagtttcatttctttaccttgaaacgtgaaagtaaatcagtgagatcagtgttgcttctgcttttaatttttttcacagttcagatttttatgtttttggttcatcactcttttaaaaatgctgtggttcccatcaaacttgttccagacttatattttctaaaacctggagacaaaccacataaatacaaaacaagtctgcttcttcaaaattctgctgactcagatatcagtgatcttattagctttttcacaagccagaacataatggtgacgatgattaatgacagtgactatgtatccgtggctccccatagtgcggctttaaatgtgatgcattcagaaaaggactatgtttttgcagctgttttcaacagtactatggtttattctttacctatattagtgaatatcattagtaactactatctttatcatttaaatgtgactgaaaccatccagatctggagtaccccattctttcaagaaattactgatatagtttttaaaattgagctgtattttcaagcagctttgcttggaatcattgttactgcaatgccaccttactttgccatggaaaatgcagagaatcataagatcaaagcttatactcaacttaaactttcaggtcttttgccatctgcatattggattggacaagctgttgttgatatccccttattttttatcattcttattttgatgctaggaagcttactggcatttcattatggattatatttttatactgtaaagttccttgctgtggttttttgccttattggttatgttccatcagttattctgttcacttatattgcttctttcacctttaagaaaattttaaataccaaagaattttggtcatttatctattctgtggcagcgttgncttgtattgcaatcactgaaataactttctttatgggatacacaattgcaactattcttcattatgccttttgtatcatcattccaatctatccacttctaggttgcctgatttctttcataaagatttcttggaagaatgtacgaaaaaatgtggacacctataatccatgggataggctttcagtagctgttatatcgccttacctgcagtgtgtactgtggattttcctcttacaatactatgagaaaaaatatggaggcagatcaataagaaaagatccctttttcagaaacctttcaacgaagtctaaaaataggaagcttccagaaccaccagacaatgaggatgaagatgaagatgtcaaagctgaaagactaaaggtcaaagagctgatgggttgccagtgttgtgaggagaaaccatccattatggtcagcaatttgcataaagaatatgatgacaagaaagattttcttctttcaagaaaagtaaagaaagtggcaactaaatacatctctttctgtgtgaaaaaaggagagatcttaggactattgggtccaaatggtgctggcaaaagcacaattattaatattctggttggtgatattgaaccaacttcaggccaggtatttttaggagattattcttcagagacaagtgaagatgatgattcactgaagtgtatgggttactgtcctcagataaaccctttgtggccagatactacattgcaggaacattttgaaatttatggagctgtcaaaggaatgagtgcaagtgacatgaaagaagtcataagtcgaataacacatgcacttgatttaaaagaacatcttcagaagactgtaaagaaactacctgcaggaatcaaacgaaagttgtgttttgctctaagtatgctagggaatcctcagattactttgctagatgaaccatctacaggtatggatcccaaagccaaacagcacatgtggcgagcaattcgaactgcatttaaaaacagaaagcgggctgctattctgaccactcactatatggaggaggcagaggctgtctgtgatcgagtagctatcatggtgtctgggcagttaagatgtatcggaacagtacaacatctaaagagtaaatttggaaaaggctactttttggaaattaaattgaaggactggatagaaaacctagaagtagaccgccttcaaagagaaattcagtatattttcccaaatgcaagccgtcaggaaagtttttcttctattttggcttataaaattcctaaggaagatgttcagtccctttcacaatctttttttaagctggaagaagctaaacatgcttttgccattgaagaatatagcttttctcaagcaacattggaacaggtttttgtagaactcactaaagaacaagaggaggaagataatagttgtggaactttaaacagcacactttggtgggaacgaacacaagaagatagagtagtattttgaatttgtattgttcggtctgcttactgggacttctttctttttcacttaattttaactttggtttaaaaagttttttattggaatggtaactggagaaccaagaacgcacttgaaatttttctaagctccttaattgaaatgctgtggttgtgtgttttgcttttctttaaataaaacgtatgtataattaagtgaagctgcatgtttgtattgaagtatattgaactatatagtttgtatgtcatctttttcaccattcagaaacagtgcttctgaatttgtgatttaaaggaattgtaatagaatagttttatttttaagttatctttaagtttatgccatcttcttaaataagtacgtaatgttccaatctaaataaaaaactaatacataactaatgcatagaaaagatacataaagcaatgtgaaagtttcttgcttctcctttttaatttctaaaaaagccactttgaatggaagttgtcatccgtaaaagctgaagtgtaagcactaggaaatctcaatatagagatttgaggaaagttatatccactaggtggcagtcattgatcataataagtgaaat

SEQ ID NO: 2 - DNA, 5296 bp, Homo sapiens
ctgctggagtaggcacccatttaaagaaaaaatgaagaagcagcaataaagaagttgtaatcgttacctagacaaacagagaactggttttgacagtgtttctagagtgctttttattattttcctgacagttgtgttccaccatgattactttctccttcagcgaataggctaaatgaatatgaaacagaaaagcgtgtatcagcaaaccaaagcacttctgtgcaagaattttcttaagaaatggaggatgaaaagagagagcttattggaatggggcctctcaatacttctaggactgtgtattgctctgttttccagttccatgagaaatgtccagtttcctggaatggctcctcagaatctgggaagggtagataaatttaatagctcttctttaatggttgtgtatacaccaatatctaatttaacccagcagataatgaataaaacagcacttgctcctcttttgaaaggaacaagtgtcattggggcaccaaataaaacacacatggacgaaatacttctggaaaatttaccatatgctatgggaatcatctttaatgaaactttctcttataagttaatatttttccagggatataacagtccactttggaaagaagatttctcagctcattgctgggatggatatggtgagttttcatgtacattgaccaaatactggaatagaggatttgtggctttacaaacagctattaatactgccattatagaaatcacaaccaatcaccctgtgatggaggagttgatgtcagttactgctataactatgaagacattacctttcataactaaaaatcttcttcacaatgagatgtttattttattcttcttgcttcatttctccccacttgtatattttatatcactcaatgtaacaaaagagagaaaaaagtctaagaatttgatgaaaatgatgggtctccaagattcagcattctggctctcctggggtctaatctatgctggcttcatctttattatttccatattcattacaattatcataacattcacccaaattatagtcatgactggcttcatggtcatatttatactcttttttttatatggcttatctttggtagctttggtgttcctgatgagtgtgctgttaaagaaagctgtcctcaccaatttggttgtgtttctccttaccctcttttggggatgtctgggattcactgtattttatgaacaacttccttcatctctggagtggattttgaatatttgtagcccttttgcctttactactggaatgattcagattatcaaactggattataacttgaatggtgtaatttttcctgacccttcaggagactcatatacaatgatagcaactttttctatgttgcttttggatggtctcatctacttgctattggcattatactttgacaaaattttaccctatggagatgagcgccattattctcctttatttttcttgaattcatcatcttgtttccaacaccaaaggactaatgctaaggttattgagaaagaaatcgatgctgagcatccctctgatgattattttgaaccagtagctcctgaattccaaggaaaagaagccatcagaatcagaaatgttaagaaggaatataaaggaaaatctggaaaagtggaagcattgaaaggcttgctctttgacatatatgaaggtcaaatcacggcaatcctgggtcacagtggagctggcaaatcttcactgctaaatattcttaatggattgtctgttccaacagaaggatcagttaccatctataataaaaatctctctgaaatgcaagacttggaggaaatcagaaagataactggcgtctgtcctcaattcaatgttcaatttgacatactcaccgtgaaggaaaacctcagcctgtttgctaaaataaaagggattcatctaaaggaagtggaacaagaggtacaacgaatattattggaattggacatgcaaaacattcaagataaccttgctaaacatttaagtgaaggacagaaaagaaagctgacttttgggattaccattttaggagatcctcaaattttgcttttagatgaaccaactactggattggatcccttttccagagatcaagtgtggagcctcctgagagagcgtagagcagatcatgtgatccttttcagtacccagtccatggatgaggctgacatcctggctgatagaaaagtgatcatgtccaatgggagactgaagtgtgcaggttcttctatgtttttgaaaagaaggtggggtcttggatatcacctaagtttacataggaatgaaatatgtaacccagaacaaataacatccttcattactcatcacatccccgatgctaaattaaaaacagaaaacaaagaaaagcttgtatatactttgccactggaaaggacaaatacatttccagatcttttcagtgatctggataagtgttctgaccagggagtgacaggttatgacatttccatgtcaactctaaatgaagtctttatgaaactggaaggacagtcaactatcgaacaagatttcgaacaagtggagatgataagagactcagaaagcctcaatgaaatggagctggctcactcttccttctctgaaatgcagacagctgtgagtgacatgggcctctggagaatgcaagtctttgccatggcacggctccgtttcttaaagttaaaacgtcaaactaaagtgttattgaccctattattggtatttggaatcgcaatattccctttgattgttgaaaatataatatatgctatgttaaatgaaaagatcgattgggaatttaaaaacgaattgtattttctctctcctggacaacttccccaggaaccccgtaccagcctgttgatcatcaataacacagaatcaaatattgaagattttataaaatcactgaagcatcaaaatatacttttggaagtagatgactttgaaaacagaaatggtactgatggcctctcatacaatggagctatcatagtttctggtaaacaaaaggattatagattttcagttgtgtgtaataccaagagattgcactgttttccaattcttatgaatattatcagcaatgggctacttcaaatgtttaatcacacacaacatattcgaattgagtcaagcccatttcctcttagccacataggactctggactgggttgccggatggttcctttttcttatttttggttctatgtagcatttctccttatatcaccatgggcagcatcagtgattacaagaaaaatgctaagtcccagctatggatttcaggcctctacacttctgcttactggtgtgggcaggcactagtggacgtcagcttcttcattttaattctccttttaatgtatttaattttctacatagaaaacatgcagtaccttcttattacaagccaaattgtgtttgctttggttatagttactcctggttatgcagcttctcttgtcttcttcatatatatgatatcatttatttttcgcaaaaggagaaaaaacagtggcctttggtcattttacttcttttttgcctccaccatcatgttttccatcactttaatcaatcattttgacctaagtatattgattaccaccatggtattggttccttcatataccttgcttggatttaaaacttttttggaagtgagagaccaggagcactacagagaatttccagaggcaaattttgaattgagtgccactgattttctagtctgcttcataccctactttcagactttgctattcgtttttgttctaagatgcatggaactaaaatgtggaaagaaaagaatgcgaaaagatcctgttttcagaatttccccccaaagtagagatgctaagccaaatccagaagaacccatagatgaagatgaagatattcaaacagaaagaataagaacagccactgctctgaccacttcaatcttagatgagaaacctgttataattgccagctgtctacacaaagaatatgcaggccagaagaaaagttgcttttcaaagaggaagaagaaaatagcagcaagaaatatctctttctgtgttcaagaaggtgaaattttgggattgctaggacccagtggtgctggaaaaagttcatctattagaatgatatctgggatcacaaagccaactgctggagaggtggaactgaaaggctgcagttcagttttgggccacctggggtactgccctcaagagaacgtgctgtggcccatgctgacgttgagggaacacctggaggtgtatgctgccgtcaaggggctcaggaaagcggacgcgaggctcgccatcgcaagattagtgagtgctttcaaactgcatgagcagctgaatgttcctgtgcagaaattaacagcaggaatcacgagaaagttgtgttttgtgctgagcctcctgggaaactcacctgtcttgctcctggatgaaccatctacgggcatagaccccacagggcagcagcaaatgtggcaggcaatccaggcagtcgttaaaaacacagagagaggtgtcctcctgaccacccataacctggctgaggcggaagccttgtgtgaccgtgtggccatcatggtgtctggaaggcttagatgcattggctccatccaacacctgaaaaacaaacttggcaaggattacattctagagctaaaagtgaaggaaacgtctcaagtgactttggtccacactgagattctgaagcttttcccacaggctgcagggcaggaaaggtattcctctttgttaacctataagctgcccgtggcagacgtttaccctctatcacagacctttcacaaattagaagcagtgaagcataactttaacctggaagaatacagcctttctcagtgcacactggagaaggtattcttagagctttctaaagaacaggaagtaggaaattttgatgaagaaattgatacaacaatgagatggaaactcctccctcattcagatgaaccttaaaacctcaaacctagtaattttttgttgatctcctataaacttatgttttatgtaataattaatagtatgtttaattttaaagatcatttaaaattaacatcaggtatattttgtaaatttagttaacaaatacataaattttaaaattattcttcctctcaaacataggggtgatagcaaacctgtgataaaggcaatacaaaatattagtaaagtcacccaaagagtcaggcactgggtattgtggaaataaaactatataaactt

SEQ ID NO: 3 - DNA, 5981 bp, Homo sapiens
attcacaatgaatgtgaaattaaaagcatgatgtagtagtgacccaaaaggaatgtgaattctcctccagaacatgcagagacccatggatgaactgtgtttctagatttttcctccagctttcctgagagaaacaggtcaaaatgagcaagagacgcatgagcgtgggtcagcaaacatgggctcttctctgcaagaactgtctcaaaaaatggagaatgaaaagacagaccttgttggaatggctcttttcatttcttctggtactgtttctgtacctatttttctccaatttacatcaagttcatgacactcctcaaatgtcttcaatggatctgggacgtgtagatagttttaatgatactaattatgttattgcatttgcacctgaatccaaaactacccaagagataatgaacaaagtggcttcagccccattcctaaaaggaagaacaatcatggggtggcctgatgaaaaaagcatggatgaattggatttgaactattcaatagacgcagtgagagtcatctttactgataccttctcctaccatttgaagttttcttggggacatagaatccccatgatgaaagagcacagagaccattcagctcactgtcaagcagtgaatgaaaaaatgaagtgtgaaggttcagagttctgggagaaaggctttgtagcttttcaagctgccattaatgctgctatcatagaaatcgcaacaaatcattcagtgatggaacagctgatgtcagttactggtgtacatatgaagatattaccttttgttgcccaaggaggagttgcaactgattttttcattttcttttgcattatttctttttctacatttatatactatgtatcagtcaatgttacacaagaaagacaatacattacgtcattgatgacaatgatgggactccgagagtcagcattctggctttcctggggtttgatgtatgctggcttcatccttatcatggccactttaatggctcttattgtaaaatctgcacaaattgtcgtcctgactggttttgtgatggtcttcaccctctttctcctctatggcctgtctttgataactttagctttcctgatgagtgtgttgataaagaaacctttccttacgggcttggttgtgtttctccttattgtcttttgggggatcctgggattcccagcattgtatacacatcttcctgcatttttggaatggactttgtgtcttcttagcccctttgccttcactgttgggatggcccagcttatacatttggactatgatgtgaattctaatgcccacttggattcttcacaaaatccatacctcataatagctactcttttcatgttggtttttgacacccttctgtatttggtattgacattatattttgacaaaattttgcccgctgaatatggacatcgatgttctcccttgtttttcctgaaatcctgtttttggtttcaacacggaagggctaatcatgtggtccttgagaatgaaacagattctgatcctacacctaatgactgttttgaaccagtgtctccagaattctgtgggaaggaagccatcagaatcaaaaatcttaaaaaagaatatgcagggaagtgtgagagagtagaagctttgaaaggtgtggtgtttgacatatatgaaggccagatcactgccctccttggtcacagtggagctggaaaaactaccctgttaaacatacttagtgggttgtcagttccaacatcaggttcagtcactgtctataatcacacactttcaagaatggctgatatagaaaatatcagcaagttcactggattttgtccacaatccaatgtgcaatttggatttctcactgtgaaagaaaacctcaggctgtttgctaaaataaaagggattttgccacatgaagtggagaaagaggtacaacgagttgtacaggaattagaaatggaaaatattcaagacatccttgctcaaaacttaagtggtggacaaaataggaaactaacttttgggattgccattttaggagatcctcaagttttgctattggatgaaccgactgctggattggatcctctttcaaggcaccgaatatggaatctcctgaaagaggggaaatcagacagagtaattctcttcagcacccagtttatagatgaggctgacattctggcggacaggaaggtgttcatatccaatgggaagctgaagtgtgcaggctcttctctgttccttaagaagaaatggggcataggctaccatttaagtttgcatctgaatgaaaggtgtgatccagagagtataacatcactggttaagcagcacatctctgatgccaaattgacagcacaaagtgaagaaaaacttgtatatattttgcctttggaaaggacaaacaaatttccagaactttacagggatcttgatagatgttctaaccaaggcattgaggattatggtgtttccataacaactttgaatgaggtgtttctgaaattagaaggaaaatcaactattgatgaatcagatattggaatttggggacaattacaaactgatggggcaaaagatataggaagccttgttgagctggaacaagttttgtcttccttccacgaaacaaggaaaacaatcagtggcgtggcgctctggaggcagcaggtctgtgcaatagcaaaagttcgcttcctaaagttaaagaaagaaagaaaaagcctgtggactatattattgctttttggtattagctttatccctcaacttttggaacatctattctacgagtcatatcagaaaagttacccgtgggaactgtctccaaatacatacttcctctcaccaggacaacaaccacaggatcctctgacccatttactggtcatcaataagacagggtcaaccattgataactttttacattcactgaggcgacagaacatagctatagaagtggatgcctttggaactagaaatggcacagatgacccatcttacaatggtgctatcattgtgtcaggtgatgaaaaggatcacagattttcaatagcatgtaatacaaaacggctgaattgctttcctgtcctcctggatgtcattagcaatggactacttggaatttttaattcgtcagaacacattcagactgacagaagcacattttttgaagagcatatggattatgagtatgggtaccgaagtaacaccttcttctggataccgatggcagcctctttcactccatacattgcaatgagcagcattggtgactacaaaaaaaaagctcattcccagctacggatttcaggcctctacccttctgcatactggtttggccaagcactggtggatgtttccctgtactttttgatcctcctgctaatgcaaataatggattatatttttagcccagaggagattatatttataattcaaaacctgttaattcaaatcctgtgtagtattggctatgtctcatctcttgttttcttgacatatgtgatttcattcatttttcgcaatgggagaaaaaatagtggcatttggtcatttttcttcttaattgtggtcatcttctcgatagttgctactgatctaaatgaatatggatttctagggctattttttggcaccatgttaatacctcccttcacattgattggctctctattcattttttctgagatttctcctgattccatggattacttaggagcttcagaatctgaaattgtatacctggcactgctaataccttaccttcattttctcatttttcttttcattctgcgatgcctagaaatgaactgcaggaagaaactaatgagaaaggatcctgtgttcagaatttctccaagaagcaacgctatttttccaaacccagaagagcctgaaggagaggaggaagatatccagatggaaagaatgagaacagtgaatgctatggctgtgcgagactttgatgagacacccgtcatcattgccagctgtctacggaaggaatatgcaggcaaaaagaaaaattgcttttctaaaaggaagaaaacaattgccacaagaaatgtctctttttgtgttaaaaaaggtgaagttataggactgttaggacacaatggagctggtaaaagtacaactattaagatgataactggagacacaaaaccaactgcaggacaggtgattttgaaagggagcggtggaggggaacccctgggcttcctggggtactgccctcaggagaatgcgctgtggcccaacctgacagtgaggcagcacctggaggtgtacgctgccgtgaaaggtctcaggaaaggggacgcaatgatcgccatcacacggttagtggatgcgctcaagctgcaggaccagctgaaggctcccgtgaagaccttgtcagagggaataaagcgaaagctgcgctttgtgctgagcatcctggggaacccgtcagtggtgcttctggatgagccgtcgaccgggatggaccccgaggggcagcagcaaatgtggcaggtgattcgggccacctttagaaacacggagaggggcgccctcctgaccacccactacatggcagaggctgaggcggtgtgtgaccgagtggccatcatggtgtcaggaaggctgagatgtattggttccatccaacacctgaaaagcaaatttggcaaagactacctgctggagatgaagctgaagaacctggcacaaatggagcccctccatgcagagatcctgaggcttttcccccaggctgctcagcaggaaaggttctcctccctgatggtctataagttgcctgttgaggatgtgcgacctttatcacaggctttcttcaaattagagatagttaaacagagtttcgacctggaggagtacagcctctcacagtctaccctggagcaggttttcctggagctctccaaggagcaggagctgggtgatcttgaagaggactttgatccctcggtgaagtggaaactcctcctgcaggaagagccttaaagctccaaataccctatatctttctttaatcctgtgactcttttaaagataatattttatagccttaatatgccttatatcagaggtggtacaaaatgcatttgaaactcatgcaataattatcctcagtagtatttcttacagtgagacaacaggcaatgtcagtgagggcgatcgtagggcataagcctaagccataccatgcagcctttgtgccagcaaccaaatcccatgtttcctactgtgttaagtttaaaaatgcatttattatagaattgtctacatttctgaggatgtcatggagaatgcttaattttctttctctgaacttcaaaatattaaatattttcttatttttttgattaaagtataaattaagacaccctattgacttccgggtaaggggagtcaattgattacccagcagcacagtatttgctttttataattccctttttaaatacttgttcttaattgactggttttccttttctgtcatttttcagagtttagattgtgagtccatgttttgtctgttgtgcctataaaggaaatttgaaatctgtatcattctactataaagacacatgcacacgtatgtttattgcagcactgtttacaatagcaaagacttggaaccaaccaaaatacccacaaatgatagaccggataaagaaaacgtgacacatatacaccatggaatactatgcagccatagaaaaggatgagttcatattcttcacagggacatggatgaagctggaaaccatcatcctcagcaaactaacacaggaacagaaaaccaaacaccgcatgttctcactcataagtgggaattgaacaatgagaatacatggacacagggaggggaacaccacaccctggggcctgttggggggatgggggctaggggagggatagcattaggagaaatacctgatgtagatgatgggttgatgggtg

SEQ ID NO: 4 - DNA, 6181 bp, Homo sapiens
  unsure 1420: n=unknown, may be a or g or c or t
aattaattttacttaggataagtgttgttattattgtttttattgttgttctgttagttactcaaaacttcattctaattgtgccctgagtttgttaaaataccatactgtatttttgtgtaacatgtaaataggcattaatttttgagaaatagaaatgtttatccttaatgtatttttaatttgctaacattgattttttattttctttcctgaaatagcttatttcctaaaatgaaagaatttattctcagatgaataatttttatatcagctattcttatcagagcaataaacaaataccaatgatgcgctcagccaacaattcattacactctctgaagagtaactggacaaggagaaaaacatagggaaaaaaccaacagaatttgttggcatgttctacacacagaccatggcttttcagaagccaagctgaataaaaacagttttaaaagaggcaaccatttgtagaggagtccttgaaggattcttcattgttttcttggacaaaaagagaccagtggatccaagtgcttcaaatacttctctcttattttcttaactctattgctctgcaatatttactttaccctgttaatgaacaggacaaaatggttaaaaaagagataagcgtgcgtcaacaaattcaggctcttctgtacaagaattttcttaaaaaatggagaataaaaagagagtttattggaatggacaataacattgtttctagggctatatttgtgcatcttttcggaacacttcagagctacccgttttcctgaacaacctcctaaagtcctgggaagcgtggatcagtttaatgactctggcctggtagtggcatatacaccagtcagtaacataacacaaaggataatgaataagatggccttggcttcctttatgaaaggaagaacagtcattgggacaccagatgaagagaccatggatatagaacttccaaaaaaataccatgaaatggtgggagttatatttagtgatactttctcatatcgcctgaagtttaattggggatatagaatcccagttataaaggagcactctgaatacacagaacactgttgggccatgcatggtgaaattttttgttacttggcaaagtactggctaaaagggtttgtagcttttcaagctgcaattaatgctgcaattatagaagtcacaacaaatcattctgtaatggaggagttgacatcagttattggaataaatatgaagataccacctttcatttctaagggagaaattatgaatgaatggtttcattttacttgcttagtttctttctcttcttttatatactttgcatcattaaatgttgcaagggaaagaggaaaatttaagaaactgatgacagtaatgggtctccgagagtcagcattctggctctcctggngattgacatacatttgcttcatcttcattatgtccatttttatggctctggtcataacatcaatctcaattgtatttcatactggcttcatggtgatattcacactctatagcttatatggcctttctttgatagcattggctttcctcatgagtgttttaataaggaaacctatgctcgctggtttggctggatttctcttcactgtattttggggatgtctgggattcactgtgttatacagacaacttcctttatctttgggatgggtattaagtcttcttagcccttttgccttcactgctggaatggcccaggttacacacctggataattacttaagtggtgttatttttcctgatccctctggggattcatacaaaatgatagccacttttttcattttggcatttgatactcttttctatttgatattcacattatattttgagcgagttttacctgataaagatggccatggggattctccattatttttccttaagtcctcattttggtccaaacatcaaaatactcatcatgaaatctttgagaatgaaataaatcctgagcattcctctgatgattcttttgaaccggtgtctccagaattccatggaaaagaagccataagaatcagaaatgttataaaagaatataatggaaagactggaaaagtagaagcattgcaaggcatattttttgacatatatgaaggacagatcactgcaatacttgggcataatggagctggtaaatcaacactgctaaacattcttagtggattgtctgtttctacagaaggatcagccactatttataatactcaactctctgaaataactgacatggaagaaattagaaagaatattggattttgtccacagttcaattttcaatttgacttcctcactgtgagagaaaacctcagggtatttgctaaaataaaagggattcagccaaaggaagtggaacaagaggtaaaaagaattataatggaattagacatgcaaagcattcaagacattattgctaaaaaattaagtggtgggcagaagagaaaactaacactagggattgccatcttaggagatcctcaggttttgctgctagatgaaccaactgctggattggatcccttttcaagacaccgagtgtggagcctcctgaaggagcataaagtagaccgacttatcctcttcagtacccaattcatggatgaggctgacatcttggctgataggaaagtatttctgtctaatgggaagttgaaatgtgcaggatcatctttgtttctgaagcgaaagtggggtattggatatcatttaagtttacacaggaatgaaatgtgtgacacagaaaaaatcacatcccttattaagcagcacattcctgatgccaagttaacaacagaaagtgaagaaaaacttgtatatagtttgcctttggaaaaaacgaacaaatttccagatctttacagtgaccttgataagtgttctgaccagggcataaggaattatgctgtttcagtgacatctctgaatgaagtattcttgaacctagaaggaaaatcagcaattgatgaaccagattttgacattgggaaacaagagaaaatacatgtgacaagaaatactggagatgagtctgaaatggaacaggttctttgttctcttcctgaaacaagaaaggctgtcagtagtgcagctctctggagacgacaaatctatgcagtggcaacacttcgcttcttaaagttaaggcgtgaaaggagagctcttttgtgtttgttactagtacttggaattgcttttatccccatcattctagagaagataatgtataaagtaactcgtgaaactcattgttgggagttttcacccagtatgtatttcctttctctggaacaaatcccgaagacgcctcttaccagcctgttaatcgttaataatacaggatcaaatattgaagacctcgtgcattcactgaagtgtcaggatatagttttggaaatagatgactttagaaacagaaatggctcagatgatccctcctacaatggagccatcatagtgtctggtgaccagaaggattacagattttctgttgcgtgtaataccaagaaattgaattgttttcctgttcttatgggaattgttagcaatgcccttatgggaatttttaacttcacggagcttattcaaacggagagcacttcattttctcgtgatgacatagtgctggatcttggttttatagatgggtccatatttttgttgttgatcacaaactgcgtttctccttttatcggcatgagcagcatcagcgattataaaaaaaatgttcaatcccagttatggatttcaggcctctggccttcagcatactggtgtggacaggctctggtggacattccattatacttcttgattctcttttcaatacatttaatttactacttcatatttctgggattccagctttcatgggaactcatgtttgttttggtggtatgcataattggttgtgcagtttctcttatattcctcacatatgtgctttcattcatctttcgcaagtggagaaaaaataatggcttttggtcttttggcttttttattatcttaatatgtgtatccacaattatggtatcaactcaatatgaaaaactcaacttaattttgtgcatgattttcataccttccttcactttgctggggtatgtcatgttattgatccagctcgactttatgagaaacttggacagtctggacaatagaataaatgaagtcaataaaaccattcttttaacaaccttaataccataccttcagagtgttattttcctttttgtcataaggtgtctggaaatgaagtatggaaatgaaataatgaataaagacccagttttcagaatctctccacggagtagagaaactcatcccaatccggaagagcccgaagaagaagatgaagatgttcaagctgaaagagtccaagcagcaaatgcactcactgctccaaacttggaggaggaaccagtcataactgcaagctgtttacacaaggaatattatgagacaaagaaaagttgcttttcaacaagaaagaagaaaatagccatcagaaatgtttccttttgtgttaaaaaaggtgaagttttgggattactaggacacaatggagctggtaaaagtacttccattaaaatgataactgggtgcacaaagccaactgcaggagtggtggtgttacaaggcagcagagcatcagtaaggcaacagcatgacaacagcctcaagttcttggggtactgccctcaggagaactcactgtggcccaagcttacaatgaaagagcacttggagttgtatgcagctgtgaaaggactgggcaaagaagatgctgctctcagtatttcacgattggtggaagctcttaagctccaggaacaacttaaggctcctgtgaaaactctatcagagggaataaagagaaagctgtgctttgtgctgagcatcctggggaacccatcagtggtgcttctagatgagccgttcaccgggatggaccccgaggggcagcagcaaatgtggcagatacttcaggctaccgttaaaaacaaggagaggggcaccctcttgaccacccattacatgtcagaggctgaggctgtgtgtgaccgtatggccatgatggtgtcaggaacgctaaggtgtattggttccattcaacatctgaaaaacaagtttggtagagattatttactagaaataaaaatgaaagaacctacccaggtggaagctctccacacagagattttgaagcttttcccacaggctgcttggcaggaaagatattcctctttaatggcgtataagttacctgtggaggatgtccaccctctatctcgggcctttttcaagttagaggcgatgaaacagaccttcaacctggaggaatacagcctctctcaggctaccttggagcaggtattcttagaactctgtaaagagcaggagctgggaaatgttgatgataaaattgatacaacagttgaatggaaacttctcccacaggaagacccttaaaatgaagaacctcctaacattcaattttaggtcctactacattgttagtttccataattctacaagaatgtttccttttacttcagttaacaaaagaaaacatttaataaacattcaataatgattacagttttcatttttaaaaatttaggatgaaggaaacaaggaaatatagggaaaagtagtagacaaaattaacaaaatcagacatgttattcatccccaacatgggtctattttgtgcttaaaaataatttaaaaatcatacaatattaggttggttttcggttattatcaataaagctaacactgagaacattttacaaataaaaatatgagttttttagcctgaacttcaaatgtatcagctatttttaaacattatttactcggattctaatttaatgtgacattgactataagaaggtctgataaactgatgaaatggcacagcataacatttaattataatgacattctgattataaaataaatgcatgtgaattttagtacatattgaagttatatggaagaagatagccataatctgtaagaaagtaccgcagttaatattttctttagccaacttatattcaatgtattttttatggatcctttttcaaaggtagtatcagtaggcatagtcattttctgtatcttttcacctcac

SEQ ID NO: 5 - protein, 1642 aa, Homo sapiens
  UNSURE 1147: Xaa=unknown, may be any amino acid
MSTAIREVGVWRQTRTLLLKNYLIKCRTKKSSVQEILFPLFFLFWLILISMMHPNKKYEEVPNIELNPMDKFTLSNLILGYTPVTNITSSIMQKVSTDHLPDVIITEEYTNEKEMLTSSLSKPSNFVGVVFKDSMSYELRFFPDMIPVSSIYMDSRAGCSKSCEAAQYWSSGFTVLQASIDAAIIQLKTNVSLWKELESTKAVIMGETAVVEIDTFPRGVILIYLVIAFSPFGYFLAIHIVAEKEKKIKEFLKIMGLHDTAFWLSWVLLYTSLIFLMSLLMAVIATASLLFPQSSSIVIFLLFFLYGLSSVFFALMLTPLFKKSKHVGIVEFFVTVAFGFIGLMIILIESFPKSLVWLFSPFCHCTFVIGIAQVMHLEDFNEGASFSNLTAGPYPLIITIIMLTLNSIFYVLLAVYLDQVIPGEFGLRRSSLYFLKPSYWSKSKRNYEELSEGNVNGNISFSEIIEPVSSEFVGKEAIRISGIQKTYRKKGENVEALRNLSFDIYEGQITALLGHSGTGKSTLMNILCGLCPPSDGFASIYGHRVSEIDEMFEARKMIGICPQLDIHFDVLTVEENLSILASIKGIPANNIIQEVQKVLLDLDMQTIKDNQAKKLSGGQKRKLSLGIAVLGNPKILLLDEPTAGMDPCSRHIVWNLLKYRKANRVTVFSTHFMDEADILADRKAVISQGMLKCVGSSMFLKSKWGIGYRLSMYIDKYCATESLSSLVKQHIPGATLLQQNDQQLVYSLPFKDMDKFSGLFSALDSHSNLGVISYGVSMTTLEDVFLKLEVEAEIDQADYSVFTQQPLEEEMDSKSFDEMEQSLLILSETKASLVSTMSLWKQQMYTIAKFHFFTLKRESKSVRSVLLLLLIFFTVQIFMFLVHHSFKNAVVPIKLVPDLYFLKPGDKPHKYKTSLLLQNSADSDISDLISFFTSQNIMVTMINDSDYVSVAPHSAALNVMHSEKDYVFAAVFNSTMVYSLPILVNIISNYYLYHLNVTETIQIWSTPFFQEITDIVFKIELYFQAALLGIIVTAMPPYFAMENAENHKIKAYTQLKLSGLLPSAYWIGQAVVDIPLFFIILILMLGSLLAFHYGLYFYTVKFLAVVFCLIGYVPSVILFTYIASFTFKKILNTKEFWSFIYSVAALXCIAITEITFFMGYTIATILHYAFCIIIPIYPLLGCLISFIKISWKNVRKNVDTYNPWDRLSVAVISPYLQCVLWIFLLQYYEKKYGGRSIRKDPFFRNLSTKSKNRKLPEPPDNEDEDEDVKAERLKVKELMGCQCCEEKPSIMVSNLHKEYDDKKDFLLSRKVKKVATKYISFCVKKGEILGLLGPNGAGKSTIINILVGDIEPTSGQVFLGDYSSETSEDDDSLKCMGYCPQINPLWPDTTLQEHFEIYGAVKGMSASDMKEVISRITHALDLKEHLQKTVKKLPAGIKRKLCFALSMLGNPQITLLDEPSTGMDPKAKQHMWRAIRTAFKNRKRAAILTTHYMEEAEAVCDRVAIMVSGQLRCIGTVQHLKSKFGKGYFLEIKLKDWIENLEVDRLQREIQYIFPNASRQESFSSILAYKIPKEDVQSLSQSFFKLEEAKHAFAIEEYSFSQATLEQVFVELTKEQEEEDNSCGTLNSTLWWERTQEDRVVF

SEQ ID NO: 6 - protein, 1617 aa, Homo sapiens [sequence omitted]

SEQ ID NO: 7 - protein, 1624 aa, Homo sapiens [sequence omitted]

SEQ ID NO: 8 - protein, 1543 aa, Homo sapiens
  UNSURE 181: Xaa=unknown, may be any amino acid
MNKMALASFMKGRTVIGTPDEETMDIELPKKYHEMVGVIFSDTFSYRLKFNWGYRIPVIKEHSEYTEHCWAMHGEIFCYLAKYWLKGFVAFQAAINAAIIEVTTNHSVMEELTSVIGINMKIPPFISKGEIMNEWFHFTCLVSFSSFIYFASLNVARERGKFKKLMTVMGLRESAFWLSWXLTYICFIFIMSIFMALVITSISIVFHTGFMVIFTLYSLYGLSLIALAFLMSVLIRKPMLAGLAGFLFTVFWGCLGFTVLYRQLPLSLGWVLSLLSPFAFTAGMAQVTHLDNYLSGVIFPDPSGDSYKMIATFFILAFDTLFYLIFTLYFERVLPDKDGHGDSPLFFLKSSFWSKHQNTHHEIFENEINPEHSSDDSFEPVSPEFHGKEAIRIRNVIKEYNGKTGKVEALQGIFFDIYEGQITAILGHNGAGKSTLLNILSGLSVSTEGSATIYNTQLSEITDMEEIRKNIGFCPQFNFQFDFLTVRENLRVFAKIKGIQPKEVEQEVKRIIMELDMQSIQDIIAKKLSGGQKRKLTLGIAILGDPQVLLLDEPTAGLDPFSRHRVWSLLKEHKVDRLILFSTQFMDEADILADRKVFLSNGKLKCAGSSLFLKRKWGIGYHLSLHRNEMCDTEKITSLIKQHIPDAKLTTESEEKLVYSLPLEKTNKFPDLYSDLDKCSDQGIRNYAVSVTSLNEVFLNLEGKSAIDEPDFDIGKQEKIHVTRNTGDESEMEQVLCSLPETRKAVSSAALWRRQIYAVATLRFLKLRRERRALLCLLLVLGIAFIPIILEKIMYKVTRETHCWEFSPSMYFLSLEQIPKTPLTSLLIVNNTGSNIEDLVHSLKCQDIVLEIDDFRNRNGSDDPSYNGAIIVSGDQKDYRFSVACNTKKLNCFPVLMGIVSNALMGIFNFTELIQTESTSFSRDDIVLDLGFIDGSIFLLLITNCVSPFIGMSSISDYKKNVQSQLWISGLWPSAYWCGQALVDIPLYFLILFSIHLIYYFIFLGFQLSWELMFVLVVCIIGCAVSLIFLTYVLSFIFRKWRKNNGFWSFGFFIILICVSTIMVSTQYEKLNLILCMIFIPSFTLLGYVMLLIQLDFMRNLDSLDNRINEVNKTILLTTLIPYLQSVIFLFVIRCLEMKYGNEIMNKDPVFRISPRSRETHPNPEEPEEEDEDVQAERVQAANALTAPNLEEEPVITASCLHKEYYETKKSCFSTRKKKIAIRNVSFCVKKGEVLGLLGHNGAGKSTSIKMITGCTKPTAGVVVLQGSRASVRQQHDNSLKFLGYCPQENSLWPKLTMKEHLELYAAVKGLGKEDAALSISRLVEALKLQEQLKAPVKTLSEGIKRKLCFVLSILGNPSVVLLDEPFTGMDPEGQQQMWQILQATVKNKERGTLLTTHYMSEAEAVCDRMAMMVSGTLRCIGSIQHLKNKFGRDYLLEIKMKEPTQVEALHTEILKLFPQAAWQERYSSLMAYKLPVEDVHPLSRAFFKLEAMKQTFNLEEYSLSQATLEQVFLELCKEQELGNVDDKIDTTVEWKLLPQEDP

SEQ ID NO: 9 - DNA, 130 bp, Homo sapiens
ctgctggagtaggcacccatttaaagaaaaaatgaagaagcagcaataaagaagttgtaatcgttacctagacaaacagagaactggttttgacagtgtttctagagtgctttttattattttcctgaca

SEQ ID NO: 10 - DNA, 141 bp, Homo sapiens
gttgtgttccaccatgattactttctccttcagcgaataggctaaatgaatatgaaacagaaaagcgtgtatcagcaaaccaaagcacttctgtgcaagaattttcttaagaaatggaggatgaaaagagagagcttattg

SEQ ID NO: 11 - DNA, 205 bp, Homo sapiens
gaatggggcctctcaatacttctaggactgtgtattgctctgttttccagttccatgagaaatgtccagtttcctggaatggctcctcagaatctgggaagggtagataaatttaatagctcttctttaatggttgtgtatacaccaatatctaatttaacccagcagataatgaataaaacagcacttgctcctcttttgaaag

SEQ ID NO: 12 - DNA, 159 bp, Homo sapiens
gaacaagtgtcattggggcaccaaataaaacacacatggacgaaatacttctggaaaatttaccatatgctatgggaatcatctttaatgaaactttctcttataagttaatatttttccagggatataacagtccactttggaaagaagatttctcag

SEQ ID NO: 13 - DNA, 104 bp, Homo sapiens
ctcattgctgggatggatatggtgagttttcatgtacattgaccaaatactggaatagaggatttgtggctttacaaacagctattaatactgccattatagaa

SEQ ID NO: 14 - DNA, 227 bp, Homo sapiens
atcacaaccaatcaccctgtgatggaggagttgatgtcagttactgctataactatgaagacattacctttcataactaaaaatcttcttcacaatgagatgtttattttattcttcttgcttcatttctccccacttgtatattttatatcactcaatgtaacaaaagagagaaaaaagtctaagaatttgatgaaaatgatgggtctccaagattcagcattctg

SEQ ID NO: 15 - DNA, 142 bp, Homo sapiens
gctctcctggggtctaatctatgctggcttcatctttattatttccatattcattacaattatcataacattcacccaaattatagtcatgactggcttcatggtcatatttatactcttttttttatatggcttatctttg

SEQ ID NO: 16 - DNA, 186 bp, Homo sapiens
gtagctttggtgttcctgatgagtgtgctgttaaagaaagctgtcctcaccaatttggttgtgtttctccttaccctcttttggggatgtctgggattcactgtattttatgaacaacttccttcatctctggagtggattttgaatatttgtagcccttttgcctttactactggaatgattcag

SEQ ID NO: 17 - DNA, 148 bp, Homo sapiens
attatcaaactggattataacttgaatggtgtaatttttcctgacccttcaggagactcatatacaatgatagcaactttttctatgttgcttttggatggtctcatctacttgctattggcattatactttgacaaaattttaccct

SEQ ID NO: 18 - DNA, 169 bp, Homo sapiens
atggagatgagcgccattattctcctttatttttcttgaattcatcatcttgtttccaacaccaaaggactaatgctaaggttattgagaaagaaatcgatgctgagcatccctctgatgattattttgaaccagtagctcctgaattccaaggaaaagaagccatcag

SEQ ID NO: 19 - DNA, 59 bp, Homo sapiens
aatcagaaatgttaagaaggaatataaaggaaaatctggaaaagtggaagcattgaaag

SEQ ID NO: 20 - DNA, 111 bp, Homo sapiens
gcttgctctttgacatatatgaaggtcaaatcacggcaatcctgggtcacagtggagctggcaaatcttcactgctaaatattcttaatggattgtctgttccaacagaag

SEQ ID NO: 21 - DNA, 176 bp, Homo sapiens
gatcagttaccatctataataaaaatctctctgaaatgcaagacttggaggaaatcagaaagataactggcgtctgtcctcaattcaatgttcaatttgacatactcaccgtgaaggaaaacctcagcctgtttgctaaaataaaagggattcatctaaaggaagtggaacaagag

SEQ ID NO: 22 - DNA, 120 bp, Homo sapiens
gtacaacgaatattattggaattggacatgcaaaacattcaagataaccttgctaaacatttaagtgaaggacagaaaagaaagctgacttttgggattaccattttaggagatcctcaa

SEQ ID NO: 23 - DNA, 139 bp, Homo sapiens
attttgcttttagatgaaccaactactggattggatcccttttccagagatcaagtgtggagcctcctgagagagcgtagagcagatcatgtgatccttttcagtacccagtccatggatgaggctgacatcctggctg

SEQ ID NO: 24 - DNA, 91 bp, Homo sapiens
atagaaaagtgatcatgtccaatgggagactgaagtgtgcaggttcttctatgtttttgaaaagaaggtggggtcttggatatcacctaag

SEQ ID NO: 25 - DNA, 140 bp, Homo sapiens
tttacataggaatgaaatatgtaacccagaacaaataacatccttcattactcatcacatccccgatgctaaattaaaaacagaaaacaaagaaaagcttgtatatactttgccactggaaaggacaaatacatttccag

SEQ ID NO: 26 - DNA, 117 bp, Homo sapiens
atcttttcagtgatctggataagtgttctgaccagggagtgacaggttatgacatttccatgtcaactctaaatgaagtctttatgaaactggaaggacagtcaactatcgaacaag

SEQ ID NO: 27 - DNA, 184 bp, Homo sapiens
atttcgaacaagtggagatgataagagactcagaaagcctcaatgaaatggagctggctcactcttccttctctgaaatgcagacagctgtgagtgacatgggcctctggagaatgcaagtctttgccatggcacggctccgtttcttaaagttaaaacgtcaaactaaagtgttattgaccct

SEQ ID NO: 28 - DNA, 167 bp, Homo sapiens
attattggtatttggaatcgcaatattccctttgattgttgaaaatataatatatgctatgttaaatgaaaagatcgattgggaatttaaaaacgaattgtattttctctctcctggacaacttccccaggaaccccgtaccagcctgttgatcatcaataacacag

SEQ ID NO: 29 - DNA, 134 bp, Homo sapiens
aatcaaatattgaagattttataaaatcactgaagcatcaaaatatacttttggaagtagatgactttgaaaacagaaatggtactgatggcctctcatacaatggagctatcatagtttctggtaaacaaaag

SEQ ID NO: 30 - DNA, 138 bp, Homo sapiens
gattatagattttcagttgtgtgtaataccaagagattgcactgttttccaattcttatgaatattatcagcaatgggctacttcaaatgtttaatcacacacaacatattcgaattgagtcaagcccatttcctctt

SEQ ID NO: 31 - DNA, 108 bp, Homo sapiens
agccacataggactctggactgggttgccggatggttcctttttcttatttttggttctatgtagcatttctccttatatcaccatgggcagcatcagtgattacaag

SEQ ID NO: 32 - DNA, 174 bp, Homo sapiens
aaaaatgctaagtcccagctatggatttcaggcctctacacttctgcttactggtgtgggcaggcactagtggacgtcagcttcttcattttaattctccttttaatgtatttaattttctacatagaaaacatgcagtaccttcttattacaagccaaattgtgtttgctttg

SEQ ID NO: 33 - DNA, 114 bp, Homo sapiens
gttatagttactcctggttatgcagcttctcttgtcttcttcatatatatgatatcatttatttttcgcaaaaggagaaaaaacagtggcctttggtcattttacttctttttt

SEQ ID NO: 34 - DNA, 120 bp, Homo sapiens
gcctccaccatcatgttttccatcactttaatcaatcattttgacctaagtatattgattaccaccatggtattggttccttcatataccttgcttggatttaaaacttttttggaagtg

SEQ ID NO: 35 - DNA, 78 bp, Homo sapiens
agagaccaggagcactacagagaatttccagaggcaaattttgaattgagtgccactgattttctagtctgcttcata

SEQ ID NO: 36 - DNA, 92 bp, Homo sapiens
ccctactttcagactttgctattcgtttttgttctaagatgcatggaactaaaatgtggaaagaaaagaatgcgaaaagatcctgttttcag

SEQ ID NO: 37 - DNA, 121 bp, Homo sapiens
aatttccccccaaagtagagatgctaagccaaatccagaagaacccatagatgaagatgaagatattcaaacagaaagaataagaacagccactgctctgaccacttcaatcttagatgag

SEQ ID NO: 38 - DNA, 118 bp, Homo sapiens
aaacctgttataattgccagctgtctacacaaagaatatgcaggccagaagaaaagttgcttttcaaagaggaagaagaaaatagcagcaagaaatatctctttctgtgttcaagaag

SEQ ID NO: 39 - DNA, 92 bp, Homo sapiens
gtgaaattttgggattgctaggacccagtggtgctggaaaaagttcatctattagaatgatatctgggatcacaaagccaactgctggagag

SEQ ID NO: 40 - DNA, 155 bp, Homo sapiens
gtggaactgaaaggctgcagttcagttttgggccacctggggtactgccctcaagagaacgtgctgtggcccatgctgacgttgagggaacacctggaggtgtatgctgccgtcaaggggctcaggaaagcggacgcgaggctcgccatcgcaag

SEQ ID NO: 41 - DNA, 76 bp, Homo sapiens
attagtgagtgctttcaaactgcatgagcagctgaatgttcctgtgcagaaattaacagcaggaatcacgagaaag

SEQ ID NO: 42 - DNA, 95 bp, Homo sapiens
ttgtgttttgtgctgagcctcctgggaaactcacctgtcttgctcctggatgaaccatctacgggcatagaccccacagggcagcagcaaatgtg

SEQ ID NO: 43 - DNA, 120 bp, Homo sapiens
gcaggcaatccaggcagtcgttaaaaacacagagagaggtgtcctcctgaccacccataacctggctgaggcggaagccttgtgtgaccgtgtggccatcatggtgtctggaaggcttag

SEQ ID NO: 44 - DNA, 141 bp, Homo sapiens
atgcattggctccatccaacacctgaaaaacaaacttggcaaggattacattctagagctaaaagtgaaggaaacgtctcaagtgactttggtccacactgagattctgaagcttttcccacaggctgcagggcaggaaag

SEQ ID NO: 45 - DNA, 80 bp, Homo sapiens
gtattcctctttgttaacctataagctgcccgtggcagacgtttaccctctatcacagacctttcacaaattagaagcag

SEQ ID NO: 46 - DNA, 56 bp, Homo sapiens
tgaagcataactttaacctggaagaatacagcctttctcagtgcacactggagaag

SEQ ID NO: 47 - DNA, 369 bp, Homo sapiens
gtattcttagagctttctaaagaacaggaagtaggaaattttgatgaagaaattgatacaacaatgagatggaaactcctccctcattcagatgaaccttaaaacctcaaacctagtaattttttgttgatctcctataaacttatgttttatgtaataattaatagtatgtttaattttaaagatcatttaaaattaacatcaggtatattttgtaaatttagttaacaaatacataaattttaaaattattcttcctctcaaacataggggtgatagcaaacctgtgataaaggcaatacaaaatattagtaaagtcacccaaagagtcaggcactgggtattgtggaaataaaactatataaactt

SEQ ID NO: 48 - DNA, 130 bp, Homo sapiens
attcacaatgaatgtgaaattaaaagcatgatgtagtagtgacccaaaaggaatgtgaattctcctccagaacatgcagagacccatggatgaactgtgtttctagatttttcctccagctttcctgaga

SEQ ID NO: 49 - DNA, 109 bp, Homo sapiens
gaaacaggtcaaaatgagcaagagacgcatgagcgtgggtcagcaaacatgggctcttctctgcaagaactgtctcaaaaaatggagaatgaaaagacagaccttgttg

SEQ ID NO: 50 - DNA, 208 bp, Homo sapiens
gaatggctcttttcatttcttctggtactgtttctgtacctatttttctccaatttacatcaagttcatgacactcctcaaatgtcttcaatggatctgggacgtgtagatagttttaatgatactaattatgttattgcatttgcacctgaatccaaaactacccaagagataatgaacaaagtggcttcagccccattcctaaaag

SEQ ID NO: 51 - DNA, 165 bp, Homo sapiens
gaagaacaatcatggggtggcctgatgaaaaaagcatggatgaattggatttgaactattcaatagacgcagtgagagtcatctttactgataccttctcctaccatttgaagttttcttggggacatagaatccccatgatgaaagagcacagagaccattcag

SEQ ID NO: 52 - DNA, 104 bp, Homo sapiens
ctcactgtcaagcagtgaatgaaaaaatgaagtgtgaaggttcagagttctgggagaaaggctttgtagcttttcaagctgccattaatgctgctatcatagaa

SEQ ID NO: 53 - DNA, 227 bp, Homo sapiens
atcgcaacaaatcattcagtgatggaacagctgatgtcagttactggtgtacatatgaagatattaccttttgttgcccaaggaggagttgcaactgattttttcattttcttttgcattatttctttttctacatttatatactatgtatcagtcaatgttacacaagaaagacaatacattacgtcattgatgacaatgatgggactccgagagtcagcattctg

SEQ ID NO: 54 - DNA, 142 bp, Homo sapiens
gctttcctggggtttgatgtatgctggcttcatccttatcatggccactttaatggctcttattgtaaaatctgcacaaattgtcgtcctgactggttttgtgatggtcttcaccctctttctcctctatggcctgtctttg

SEQ ID NO: 55 - DNA, 186 bp, Homo sapiens
ataactttagctttcctgatgagtgtgttgataaagaaacctttccttacgggcttggttgtgtttctccttattgtcttttgggggatcctgggattcccagcattgtatacacatcttcctgcatttttggaatggactttgtgtcttcttagcccctttgccttcactgttgggatggcccag

SEQ ID NO: 56 - DNA, 148 bp, Homo sapiens
cttatacatttggactatgatgtgaattctaatgcccacttggattcttcacaaaatccatacctcataatagctactcttttcatgttggtttttgacacccttctgtatttggtattgacattatattttgacaaaattttgcccg

SEQ ID NO: 57 - DNA, 169 bp, Homo sapiens
ctgaatatggacatcgatgttctcccttgtttttcctgaaatcctgtttttggtttcaacacggaagggctaatcatgtggtccttgagaatgaaacagattctgatcctacacctaatgactgttttgaaccagtgtctccagaattctgtgggaaggaagccatcag

SEQ ID NO: 58 - DNA, 59 bp, Homo sapiens
aatcaaaaatcttaaaaaagaatatgcagggaagtgtgagagagtagaagctttgaaag

SEQ ID NO: 59 - DNA, 111 bp, Homo sapiens
gtgtggtgtttgacatatatgaaggccagatcactgccctccttggtcacagtggagctggaaaaactaccctgttaaacatacttagtgggttgtcagttccaacatcag

SEQ ID NO: 60 - DNA, 176 bp, Homo sapiens
gttcagtcactgtctataatcacacactttcaagaatggctgatatagaaaatatcagcaagttcactggattttgtccacaatccaatgtgcaatttggatttctcactgtgaaagaaaacctcaggctgtttgctaaaataaaagggattttgccacatgaagtggagaaagag

SEQ ID NO: 61 - DNA, 120 bp, Homo sapiens
gtacaacgagttgtacaggaattagaaatggaaaatattcaagacatccttgctcaaaacttaagtggtggacaaaataggaaactaacttttgggattgccattttaggagatcctcaa

SEQ ID NO: 62 - DNA, 139 bp, Homo sapiens
gttttgctattggatgaaccgactgctggattggatcctctttcaaggcaccgaatatggaatctcctgaaagaggggaaatcagacagagtaattctcttcagcacccagtttatagatgaggctgacattctggcgg

SEQ ID NO: 63 - DNA, 91 bp, Homo sapiens
acaggaaggtgttcatatccaatgggaagctgaagtgtgcaggctcttctctgttccttaagaagaaatggggcataggctaccatttaag

SEQ ID NO: 64 - DNA, 140 bp, Homo sapiens
tttgcatctgaatgaaaggtgtgatccagagagtataacatcactggttaagcagcacatctctgatgccaaattgacagcacaaagtgaagaaaaacttgtatatattttgcctttggaaaggacaaacaaatttccag

SEQ ID NO: 65 - DNA, 120 bp, Homo sapiens
aactttacagggatcttgatagatgttctaaccaaggcattgaggattatggtgtttccataacaactttgaatgaggtgtttctgaaattagaaggaaaatcaactattgatgaatcag

SEQ ID NO: 66 - DNA, 199 bp, Homo sapiens
atattggaatttggggacaattacaaactgatggggcaaaagatataggaagccttgttgagctggaacaagttttgtcttccttccacgaaacaaggaaaacaatcagtggcgtggcgctctggaggcagcaggtctgtgcaatagcaaaagttcgcttcctaaagttaaagaaagaaagaaaaagcctgtggactat

SEQ ID NO: 67 - DNA, 167 bp, Homo sapiens
attattgctttttggtattagctttatccctcaacttttggaacatctattctacgagtcatatcagaaaagttacccgtgggaactgtctccaaatacatacttcctctcaccaggacaacaaccacaggatcctctgacccatttactggtcatcaataagacag

SEQ ID NO: 68 - DNA, 134 bp, Homo sapiens
ggtcaaccattgataactttttacattcactgaggcgacagaacatagctatagaagtggatgcctttggaactagaaatggcacagatgacccatcttacaatggtgctatcattgtgtcaggtgatgaaaag

SEQ ID NO: 69 - DNA, 138 bp, Homo sapiens
gatcacagattttcaatagcatgtaatacaaaacggctgaattgctttcctgtcctcctggatgtcattagcaatggactacttggaatttttaattcgtcagaacacattcagactgacagaagcacattttttgaa

SEQ ID NO: 70 - DNA, 108 bp, Homo sapiens
gagcatatggattatgagtatgggtaccgaagtaacaccttcttctggataccgatggcagcctctttcactccatacattgcaatgagcagcattggtgactacaaa

SEQ ID NO: 71 - DNA, 174 bp, Homo sapiens
aaaaaagctcattcccagctacggatttcaggcctctacccttctgcatactggtttggccaagcactggtggatgtttccctgtactttttgatcctcctgctaatgcaaataatggattatatttttagcccagaggagattatatttataattcaaaacctgttaattcaa

SEQ ID NO: 72 - DNA, 114 bp, Homo sapiens
atcctgtgtagtattggctatgtctcatctcttgttttcttgacatatgtgatttcattcatttttcgcaatgggagaaaaaatagtggcatttggtcatttttcttcttaatt

SEQ ID NO: 73 - DNA, 120 bp, Homo sapiens
gtggtcatcttctcgatagttgctactgatctaaatgaatatggatttctagggctattttttggcaccatgttaatacctcccttcacattgattggctctctattcattttttctgag

SEQ ID NO: 74 - DNA, 69 bp, Homo sapiens
atttctcctgattccatggattacttaggagcttcagaatctgaaattgtatacctggcactgctaata

SEQ ID NO: 75 - DNA, 92 bp, Homo sapiens
ccttaccttcattttctcatttttcttttcattctgcgatgcctagaaatgaactgcaggaagaaactaatgagaaaggatcctgtgttcag

SEQ ID NO: 76 - DNA, 121 bp, Homo sapiens
aatttctccaagaagcaacgctatttttccaaacccagaagagcctgaaggagaggaggaagatatccagatggaaagaatgagaacagtgaatgctatggctgtgcgagactttgatgag

SEQ ID NO: 77 - DNA, 118 bp, Homo sapiens
acacccgtcatcattgccagctgtctacggaaggaatatgcaggcaaaaagaaaaattgcttttctaaaaggaagaaaacaattgccacaagaaatgtctctttttgtgttaaaaaag

SEQ ID NO: 78 - DNA, 92 bp, Homo sapiens
gtgaagttataggactgttaggacacaatggagctggtaaaagtacaactattaagatgataactggagacacaaaaccaactgcaggacag

SEQ ID NO: 79 - DNA, 161 bp, Homo sapiens
gtgattttgaaagggagcggtggaggggaacccctgggcttcctggggtactgccctcaggagaatgcgctgtggcccaacctgacagtgaggcagcacctggaggtgtacgctgccgtgaaaggtctcaggaaaggggacgcaatgatcgccatcacacg

SEQ ID NO: 80 - DNA, 76 bp, Homo sapiens
gttagtggatgcgctcaagctgcaggaccagctgaaggctcccgtgaagaccttgtcagagggaataaagcgaaag

SEQ ID NO: 81 - DNA, 95 bp, Homo sapiens
ctgcgctttgtgctgagcatcctggggaacccgtcagtggtgcttctggatgagccgtcgaccgggatggaccccgaggggcagcagcaaatgtg

SEQ ID NO: 82 - DNA, 120 bp, Homo sapiens
gcaggtgattcgggccacctttagaaacacggagaggggcgccctcctgaccacccactacatggcagaggctgaggcggtgtgtgaccgagtggccatcatggtgtcaggaaggctgag

SEQ ID NO: 83 - DNA, 141 bp, Homo sapiens
atgtattggttccatccaacacctgaaaagcaaatttggcaaagactacctgctggagatgaagctgaagaacctggcacaaatggagcccctccatgcagagatcctgaggcttttcccccaggctgctcagcaggaaag

SEQ ID NO: 84 - DNA, 80 bp, Homo sapiens
gttctcctccctgatggtctataagttgcctgttgaggatgtgcgacctttatcacaggctttcttcaaattagagatag

SEQ ID NO: 85 - DNA, 56 bp, Homo sapiens
ttaaacagagtttcgacctggaggagtacagcctctcacagtctaccctggagcag

SEQ ID NO: 86 - DNA, 1062 bp, Homo sapiens
gttttcctggagctctccaaggagcaggagctgggtgatcttgaagaggactttgatccctcggtgaagtggaaactcctcctgcaggaagagccttaaagctccaaataccctatatctttctttaatcctgtgactcttttaaagataatattttatagccttaatatgccttatatcagaggtggtacaaaatgcatttgaaactcatgcaataattatcctcagtagtatttcttacagtgagacaacaggcaatgtcagtgagggcgatcgtagggcataagcctaagccataccatgcagcctttgtgccagcaaccaaatcccatgtttcctactgtgttaagtttaaaaatgcatttattatagaattgtctacatttctgaggatgtcatggagaatgcttaattttctttctctgaacttcaaaatattaaatattttcttatttttttgattaaagtataaattaagacaccctattgacttccgggtaaggggagtcaattgattacccagcagcacagtatttgctttttataattccctttttaaatacttgttcttaattgactggttttccttttctgtcatttttcagagtttagattgtgagtccatgttttgtctgttgtgcctataaaggaaatttgaaatctgtatcattctactataaagacacatgcacacgtatgtttattgcagcactgtttacaatagcaaagacttggaaccaaccaaaatacccacaaatgatagaccggataaagaaaacgtgacacatatacaccatggaatactatgcagccatagaaaaggatgagttcatattcttcacagggacatggatgaagctggaaaccatcatcctcagcaaactaacacaggaacagaaaaccaaacaccgcatgttctcactcataagtgggaattgaacaatgagaatacatggacacagggaggggaacaccacaccctggggcctgttggggggatgggggctaggggagggatagcattaggagaaatacctgatgtagatgatgggttgatgggtg

SEQ ID NO: 87 - DNA, 287 bp, Homo sapiens
aattaattttacttaggataagtgttgttattattgtttttattgttgttctgttagttactcaaaacttcattctaattgtgccctgagtttgttaaaataccatactgtatttttgtgtaacatgtaaataggcattaatttttgagaaatagaaatgtttatccttaatgtatttttaatttgctaacattgattttttattttctttcctgaaatagcttatttcctaaaatgaaagaatttattctcagatgaataatttttatatcagctattcttatcag

SEQ ID NO: 88 - DNA, 280 bp, Homo sapiens
agcaataaacaaataccaatgatgcgctcagccaacaattcattacactctctgaagagtaactggacaaggagaaaaacatagggaaaaaaccaacagaatttgttggcatgttctacacacagaccatggcttttcagaagccaagctgaataaaaacagttttaaaagaggcaaccatttgtagaggagtccttgaaggattcttcattgttttcttggacaaaaagagaccagtggatccaagtgcttcaaatacttctctcttattttcttaact

SEQ ID NO: 89 - DNA, 141 bp, Homo sapiens
ctattgctctgcaatatttactttaccctgttaatgaacaggacaaaatggttaaaaaagagataagcgtgcgtcaacaaattcaggctcttctgtacaagaattttcttaaaaaatggagaataaaaagagagtttattg

SEQ ID NO: 90 - DNA, 205 bp, Homo sapiens
gaatggacaataacattgtttctagggctatatttgtgcatcttttcggaacacttcagagctacccgttttcctgaacaacctcctaaagtcctgggaagcgtggatcagtttaatgactctggcctggtagtggcatatacaccagtcagtaacataacacaaaggataatgaataagatggccttggcttcctttatgaaag

SEQ ID NO: 91 - DNA, 165 bp, Homo sapiens
gaagaacagtcattgggacaccagatgaagagaccatggatatagaacttccaaaaaaataccatgaaatggtgggagttatatttagtgatactttctcatatcgcctgaagtttaattggggatatagaatcccagttataaaggagcactctgaatacacag

SEQ ID NO: 92 - DNA, 104 bp, Homo sapiens
aacactgttgggccatgcatggtgaaattttttgttacttggcaaagtactggctaaaagggtttgtagcttttcaagctgcaattaatgctgcaattatagaa

SEQ ID NO: 93 - DNA, 227 bp, Homo sapiens
gtcacaacaaatcattctgtaatggaggagttgacatcagttattggaataaatatgaagataccacctttcatttctaagggagaaattatgaatgaatggtttcattttacttgcttagtttctttctcttcttttatatactttgcatcattaaatgttgcaagggaaagaggaaaatttaagaaactgatgacagtaatgggtctccgagagtcagcattctg

SEQ ID NO: 94 - DNA, 142 bp, Homo sapiens
  unsure 11: n=unknown, may be a or g or c or t
gctctcctggngattgacatacatttgcttcatcttcattatgtccatttttatggctctggtcataacatcaatctcaattgtatttcatactggcttcatggtgatattcacactctatagcttatatggcctttctttg

SEQ ID NO: 95 - DNA, 186 bp, Homo sapiens
atagcattggctttcctcatgagtgttttaataaggaaacctatgctcgctggtttggctggatttctcttcactgtattttggggatgtctgggattcactgtgttatacagacaacttcctttatctttgggatgggtattaagtcttcttagcccttttgccttcactgctggaatggcccag

SEQ ID NO: 96 - DNA, 148 bp, Homo sapiens
gttacacacctggataattacttaagtggtgttatttttcctgatccctctggggattcatacaaaatgatagccacttttttcattttggcatttgatactcttttctatttgatattcacattatattttgagcgagttttacctg

SEQ ID NO: 97 - DNA, 169 bp, Homo sapiens
ataaagatggccatggggattctccattatttttccttaagtcctcattttggtccaaacatcaaaatactcatcatgaaatctttgagaatgaaataaatcctgagcattcctctgatgattcttttgaaccggtgtctccagaattccatggaaaagaagccataag

SEQ ID NO: 98 - DNA, 59 bp, Homo sapiens
aatcagaaatgttataaaagaatataatggaaagactggaaaagtagaagcattgcaag

SEQ ID NO: 99 - DNA, 111 bp, Homo sapiens
gcatattttttgacatatatgaaggacagatcactgcaatacttgggcataatggagctggtaaatcaacactgctaaacattcttagtggattgtctgtttctacagaag

SEQ ID NO: 100 - DNA, 176 bp, Homo sapiens
gatcagccactatttataatactcaactctctgaaataactgacatggaagaaattagaaagaatattggattttgtccacagttcaattttcaatttgacttcctcactgtgagagaaaacctcagggtatttgctaaaataaaagggattcagccaaaggaagtggaacaagag

SEQ ID NO: 101 - DNA, 120 bp, Homo sapiens
gtaaaaagaattataatggaattagacatgcaaagcattcaagacattattgctaaaaaattaagtggtgggcagaagagaaaactaacactagggattgccatcttaggagatcctcag

SEQ ID NO: 102 - DNA, 139 bp, Homo sapiens
gttttgctgctagatgaaccaactgctggattggatcccttttcaagacaccgagtgtggagcctcctgaaggagcataaagtagaccgacttatcctcttcagtacccaattcatggatgaggctgacatcttggctg

SEQ ID NO: 103 - DNA, 91 bp, Homo sapiens
ataggaaagtatttctgtctaatgggaagttgaaatgtgcaggatcatctttgtttctgaagcgaaagtggggtattggatatcatttaag

SEQ ID NO: 104 - DNA, 140 bp, Homo sapiens
tttacacaggaatgaaatgtgtgacacagaaaaaatcacatcccttattaagcagcacattcctgatgccaagttaacaacagaaagtgaagaaaaacttgtatatagtttgcctttggaaaaaacgaacaaatttccag

SEQ ID NO: 105 - DNA, 120 bp, Homo sapiens
atctttacagtgaccttgataagtgttctgaccagggcataaggaattatgctgtttcagtgacatctctgaatgaagtattcttgaacctagaaggaaaatcagcaattgatgaaccag

SEQ ID NO: 106 - DNA, 199 bp, Homo sapiens
attttgacattgggaaacaagagaaaatacatgtgacaagaaatactggagatgagtctgaaatggaacaggttctttgttctcttcctgaaacaagaaaggctgtcagtagtgcagctctctggagacgacaaatctatgcagtggcaacacttcgcttcttaaagttaaggcgtgaaaggagagctcttttgtgttt

SEQ ID NO: 107 - DNA, 167 bp, Homo sapiens
gttactagtacttggaattgcttttatccccatcattctagagaagataatgtataaagtaactcgtgaaactcattgttgggagttttcacccagtatgtatttcctttctctggaacaaatcccgaagacgcctcttaccagcctgttaatcgttaataatacag

SEQ ID NO: 108 - DNA, 134 bp, Homo sapiens
gatcaaatattgaagacctcgtgcattcactgaagtgtcaggatatagttttggaaatagatgactttagaaacagaaatggctcagatgatccctcctacaatggagccatcatagtgtctggtgaccagaag

SEQ ID NO: 109 - DNA, 138 bp, Homo sapiens
gattacagattttctgttgcgtgtaataccaagaaattgaattgttttcctgttcttatgggaattgttagcaatgcccttatgggaatttttaacttcacggagcttattcaaacggagagcacttcattttctcgt

SEQ ID NO: 110 - DNA, 108 bp, Homo sapiens
gatgacatagtgctggatcttggttttatagatgggtccatatttttgttgttgatcacaaactgcgtttctccttttatcggcatgagcagcatcagcgattataaa

SEQ ID NO: 111 - DNA, 171 bp, Homo sapiens
aaaaatgttcaatcccagttatggatttcaggcctctggccttcagcatactggtgtggacaggctctggtggacattccattatacttcttgattctcttttcaatacatttaatttactacttcatatttctgggattccagctttcatgggaactcatgtttgttttg

SEQ ID NO: 112 - DNA, 114 bp, Homo sapiens
gtggtatgcataattggttgtgcagtttctcttatattcctcacatatgtgctttcattcatctttcgcaagtggagaaaaaataatggcttttggtcttttggcttttttatt

SEQ ID NO: 113 - DNA, 120 bp, Homo sapiens
atcttaatatgtgtatccacaattatggtatcaactcaatatgaaaaactcaacttaattttgtgcatgattttcataccttccttcactttgctggggtatgtcatgttattgatccag

SEQ ID NO: 114 - DNA, 81 bp, Homo sapiens
ctcgactttatgagaaacttggacagtctggacaatagaataaatgaagtcaataaaaccattcttttaacaaccttaata

SEQ ID NO: 115 - DNA, 92 bp, Homo sapiens
ccataccttcagagtgttattttcctttttgtcataaggtgtctggaaatgaagtatggaaatgaaataatgaataaagacccagttttcag

SEQ ID NO: 116 - DNA, 121 bp, Homo sapiens
aatctctccacggagtagagaaactcatcccaatccggaagagcccgaagaagaagatgaagatgttcaagctgaaagagtccaagcagcaaatgcactcactgctccaaacttggaggag

SEQ ID NO: 117 - DNA, 118 bp, Homo sapiens
gaaccagtcataactgcaagctgtttacacaaggaatattatgagacaaagaaaagttgcttttcaacaagaaagaagaaaatagccatcagaaatgtttccttttgtgttaaaaaag

SEQ ID NO: 118 - DNA, 92 bp, Homo sapiens
gtgaagttttgggattactaggacacaatggagctggtaaaagtacttccattaaaatgataactgggtgcacaaagccaactgcaggagtg

SEQ ID NO: 119 - DNA, 179 bp, Homo sapiens
gtggtgttacaaggcagcagagcatcagtaaggcaacagcatgacaacagcctcaagttcttggggtactgccctcaggagaactcactgtggcccaagcttacaatgaaagagcacttggagttgtatgcagctgtgaaaggactgggcaaagaagatgctgctctcagtatttcacg

SEQ ID NO: 120 - DNA, 76 bp, Homo sapiens
attggtggaagctcttaagctccaggaacaacttaaggctcctgtgaaaactctatcagagggaataaagagaaag

SEQ ID NO: 121 - DNA, 95 bp, Homo sapiens
ctgtgctttgtgctgagcatcctggggaacccatcagtggtgcttctagatgagccgttcaccgggatggaccccgaggggcagcagcaaatgtg

SEQ ID NO: 122 - DNA, 120 bp, Homo sapiens
gcagatacttcaggctaccgttaaaaacaaggagaggggcaccctcttgaccacccattacatgtcagaggctgaggctgtgtgtgaccgtatggccatgatggtgtcaggaacgctaag

SEQ ID NO: 123 - DNA, 141 bp, Homo sapiens
gtgtattggttccattcaacatctgaaaaacaagtttggtagagattatttactagaaataaaaatgaaagaacctacccaggtggaagctctccacacagagattttgaagcttttcccacaggctgcttggcaggaaag

SEQ ID NO: 124 - DNA, 80 bp, Homo sapiens
atattcctctttaatggcgtataagttacctgtggaggatgtccaccctctatctcgggcctttttcaagttagaggcga

SEQ ID NO: 125 - DNA, 56 bp, Homo sapiens
tgaaacagaccttcaacctggaggaatacagcctctctcaggctaccttggagcag

SEQ ID NO: 126 - DNA, 769 bp, Homo sapiens
gtattcttagaactctgtaaagagcaggagctgggaaatgttgatgataaaattgatacaacagttgaatggaaacttctcccacaggaagacccttaaaatgaagaacctcctaacattcaattttaggtcctactacattgttagtttccataattctacaagaatgtttccttttacttcagttaacaaaagaaaacatttaataaacattcaataatgattacagttttcatttttaaaaatttaggatgaaggaaacaaggaaatatagggaaaagtagtagacaaaattaacaaaatcagacatgttattcatccccaacatgggtctattttgtgcttaaaaataatttaaaaatcatacaatattaggttggttttcggttattatcaataaagctaacactgagaacattttacaaataaaaatatgagttttttagcctgaacttcaaatgtatcagctatttttaaacattatttactcggattctaatttaatgtgacattgactataagaaggtctgataaactgatgaaatggcacagcataacatttaattataatgacattctgattataaaataaatgcatgtgaattttagtacatattgaagttatatggaagaagatagccataatctgtaagaaagtaccgcagttaatattttctttagccaacttatattcaatgtattttttatggatcctttttcaaaggtagtatcagtaggcatagtcattttctgtatcttttcacctcac

SEQ ID NO: 127 - DNA, 19 bp, Homo sapiens
cagtgactatgtatccgtg

SEQ ID NO: 128 - DNA, 19 bp, Homo sapiens
gatggtttctcctcacaac

SEQ ID NO: 129 - DNA, 19 bp, Homo sapiens
caccagacaatgaggatga

SEQ ID NO: 130 - DNA, 19 bp, Homo sapiens
gctatattcttcaatggca

SEQ ID NO: 131 - DNA, 19 bp, Homo sapiens
cctagaagtagaccgcctt

SEQ ID NO: 132 - DNA, 19 bp, Homo sapiens
gttgtgaggagaaaccatc

SEQ ID NO: 133 - DNA, 19 bp, Homo sapiens
ctggatggtttcagtcaca

SEQ ID NO: 134 - DNA, 19 bp, Homo sapiens
cagaaaagccaatcgggtg

SEQ ID NO: 135 - DNA, 23 bp, Homo sapiens
ccaggtatatgttgtttaaccag

SEQ ID NO: 136 - DNA, 20 bp, Homo sapiens
gggtcagattactgccttac

SEQ ID NO: 137 - DNA, 20 bp, Homo sapiens
gaacattgaagaaccaacac

SEQ ID NO: 138 - DNA, 20 bp, Homo sapiens
gtaaggcagtaatctgaccc

SEQ ID NO: 139 - DNA, 18 bp, Homo sapiens
ggaaactggacagaatgc

SEQ ID NO: 140 - DNA, 19 bp, Homo sapiens
ctaccctatttcacatgcc

SEQ ID NO: 141 - DNA, 20 bp, Homo sapiens
gtttctcccataataacagc

SEQ ID NO: 142 - DNA, 20 bp, Homo sapiens
gctgttattatgggagaaac

SEQ ID NO: 143 - DNA, 30 bp, Homo sapiens
agactacagtaacaaaagcctagtgcagcc

SEQ ID NO: 144 - DNA, 30 bp, Homo sapiens
atccaatcctattagtgtgacaaaggcttg

SEQ ID NO: 145 - DNA, 20 bp, Homo sapiens
tcagcaaaccaaagcacttc

SEQ ID NO: 146 - DNA, 27 bp, Homo sapiens
caagtgctgttttattcattatctgct

SEQ ID NO: 147 - DNA, 28 bp, Homo sapiens
gtacatgaaaactcaccatatccatccc

SEQ ID NO: 148 - DNA, 20 bp, Homo sapiens
tcattgctgggatggatatg

SEQ ID NO: 149 - DNA, 19 bp, Homo sapiens
ccctgtgatggaggagttg

SEQ ID NO: 150 - DNA, 21 bp, Homo sapiens
tgacatcaactcctccatcac

SEQ ID NO: 151 - DNA, 20 bp, Homo sapiens
gaatgctgaatcttggagac

SEQ ID NO: 152 - DNA, 20 bp, Homo sapiens
gattcagattatcaaactgg

SEQ ID NO: 153 - DNA, 21 bp, Homo sapiens
tggtgtaatttttcctgaccc

SEQ ID NO: 154 - DNA, 22 bp, Homo sapiens
aagggtcaggaaaaattacacc

SEQ ID NO: 155 - DNA, 19 bp, Homo sapiens
ggaattcaggagctactgg

SEQ ID NO: 156 - DNA, 22 bp, Homo sapiens
gattgtctgttccaacagaagg

SEQ ID NO: 157 - DNA, 22 bp, Homo sapiens
ccacttcctttagatgaatccc

SEQ ID NO: 158 - DNA, 22 bp, Homo sapiens
aagtggaacaagaggtacaacg

SEQ ID NO: 159 - DNA, 21 bp, Homo sapiens
atggtaatcccaaaagtcagc

SEQ ID NO: 160 - DNA, 22 bp, Homo sapiens
ggggatgtgatgagtaatgaag

SEQ ID NO: 161 - DNA, 22 bp, Homo sapiens
cttcattactcatcacatcccc

SEQ ID NO: 162 - DNA, 19 bp, Homo sapiens
acaacttccccaggaaccc

SEQ ID NO: 163 - DNA, 19 bp, Homo sapiens
gatcaacaggctggtacgg

SEQ ID NO: 164 - DNA, 22 bp, Homo sapiens
caagaaaaatgctaagtcccag

SEQ ID NO: 165 - DNA, 19 bp, Homo sapiens
tgcccacaccagtaagcag

SEQ ID NO: 166 - DNA, 22 bp, Homo sapiens
gaaaatcagtggcactcaattc

SEQ ID NO: 167 - DNA, 22 bp, Homo sapiens
tgccactgattttctagtctgc

SEQ ID NO: 168 - DNA, 19 bp, Homo sapiens
ctgggatcacaaagccaac

SEQ ID NO: 169 - DNA, 20 bp, Homo sapiens
cctttcagttccacctctcc

SEQ ID NO: 170 - DNA, 21 bp, Homo sapiens
tccacactgagattctgaagc

SEQ ID NO: 171 - DNA, 19 bp, Homo sapiens
aatacctttcctgccctgc

SEQ ID NO: 172 - DNA, 19 bp, Homo sapiens
gcctgactctttgggtgac

SEQ ID NO: 173 - DNA, 19 bp, Homo sapiens
tgagcgtgggtcagcaaac

SEQ ID NO: 174 - DNA, 20 bp, Homo sapiens
gcaactcctccttgggcaac

SEQ ID NO: 175 - DNA, 19 bp, Homo sapiens
tttgttgcccaaggaggag

SEQ ID NO: 176 - DNA, 22 bp, Homo sapiens
ggaaaaacaagggagaacatcg

SEQ ID NO: 177 - DNA, 19 bp, Homo sapiens
gcccacttggattcttcac

SEQ ID NO: 178 - DNA, 22 bp, Homo sapiens
ccacacctttcaaagcttctac

SEQ ID NO: 179 - DNA, 22 bp, Homo sapiens
atgtggtccttgagaatgaaac

SEQ ID NO: 180 - DNA, 22 bp, Homo sapiens
actgtgaaagaaaacctcaggc

SEQ ID NO: 181 - DNA, 19 bp, Homo sapiens
cttcatgtggcaaaatccc

SEQ ID NO: 182 - DNA, 20 bp, Homo sapiens
tgtgctgtcaatttggcatc

SEQ ID NO: 183 - DNA, 20 bp, Homo sapiens
aagaagaaatggggcatagg

SEQ ID NO: 184 - DNA, 22 bp, Homo sapiens
tgtatttggagacagttcccac

SEQ ID NO: 185 - DNA, 19 bp, Homo sapiens
aacaatcagtggcgtggcg

SEQ ID NO: 186 - DNA, 22 bp, Homo sapiens
gacatccaggaggacaggaaag

SEQ ID NO: 187 - DNA, 21 bp, Homo sapiens
gcagcctctttcactccatac

SEQ ID NO: 188 - DNA, 22 bp, Homo sapiens
cattgtgtcaggtgatgaaaag

SEQ ID NO: 189 - DNA, 20 bp, Homo sapiens
ttcatttctaggcatcgcag

SEQ ID NO: 190 - DNA, 22 bp, Homo sapiens
cattagcaggaggatcaaaaag

SEQ ID NO: 191 - DNA, 21 bp, Homo sapiens
tctagggctattttttggcac

SEQ ID NO: 192 - DNA, 19 bp, Homo sapiens
cgctccctttcaaaatcac

SEQ ID NO: 193 - DNA, 21 bp, Homo sapiens
tgcgagactttgatgagacac

SEQ ID NO: 194 - DNA, 20 bp, Homo sapiens
agaccatcagggaggagaac

SEQ ID NO: 195 - DNA, 18 bp, Homo sapiens
tgtgccagcaaccaaatc

SEQ ID NO: 196 - DNA, 22 bp, Homo sapiens
gctggagatgaagctgaagaac

SEQ ID NO: 197 - DNA, 18 bp, Homo sapiens
tttccacttcaccgaggg

SEQ ID NO: 198 - DNA, 21 bp, Homo sapiens
ccatgttttgtctgttgtgcc

SEQ ID NO: 199 - DNA, 22 bp, Homo sapiens
cacccatcaacccatcatctac

SEQ ID NO: 200 - DNA, 22 bp, Homo sapiens
aggcacaacagacaaaacatgg

SEQ ID NO: 201 - DNA, 22 bp, Homo sapiens
aagcatgatgtagtagtgaccc

SEQ ID NO: 202 - DNA, 25 bp, Homo sapiens
cttgggtagttttggattcaggtgc

SEQ ID NO: 203 - DNA, 27 bp, Homo sapiens
agatccattgaagacatttgaggagtg

SEQ ID NO: 204 - DNA, 20 bp, Homo sapiens
gattgacatacatttgcttc

SEQ ID NO: 205 - DNA, 20 bp, Homo sapiens
tacagtgaagagaaatccag

SEQ ID NO: 206 - DNA, 18 bp, Homo sapiens
tggaattagacatgcaaa

SEQ ID NO: 207 - DNA, 19 bp, Homo sapiens
tgaagaggataagtcggtc

SEQ ID NO: 208 - DNA, 18 bp, Homo sapiens
tataatcgctgatgctgc

SEQ ID NO: 209 - DNA, 26 bp, Homo sapiens
accaggccagagtcattaaactgatc

SEQ ID NO: 210 - DNA, 25 bp, Homo sapiens
ccgaaaagatgcacaaatatagccc

SEQ ID NO: 211 - DNA, 26 bp, Homo sapiens
ctcaaaacttcattctaattgtgccc

SEQ ID NO: 212 - DNA, 18 bp, Homo sapiens
agataagcgtgcgtcaac

SEQ ID NO: 213 - DNA, 20 bp, Homo sapiens
tcttatgggaattgttagca

SEQ ID NO: 214 - DNA, 19 bp, Homo sapiens
ttatgactggttcctcctc

SEQ ID NO: 215 - DNA, 18 bp, Homo sapiens
tcatcaacatttcccagc

SEQ ID NO: 216 - DNA, 21 bp, Homo sapiens
gaaatactggagatgagtctg

SEQ ID NO: 217 - DNA, 19 bp, Homo sapiens
gagcttaagagcttccacc

(19) United States
(12) Patent Application Publication
(10) Pub. No.: US 2003/0044895 A1
(43) Pub. Date: Mar. 6, 2003

(54) Nucleic acids of the human ABCA5, ABCA6, ABCA9, AND ABCA10 Genes, vectors containing such nucleic acids, and uses thereof

(76) Inventors: Patrice Denefle, Saint Maur (FR); Marie-Francoise Rosier-Montus, Antony (FR); Catherine Prades, Thiais (FR); Isabelle Arnould-Reguigne, Chennevieres Sur Marne (FR); Nicolas Duverger, Paris (FR); Rando Allikmets, Cornwall-on Hudson, NY (US); Michael Dean, Frederick, MD (US)

Correspondence Address: Finnegan, Henderson, Farabow,, Garrett & Dunner, L.L.P, 1300 I Street, N.W., Washington, DC, 20005, US

(21) Appl. No.: 10/005,338

(22) Filed: Dec. 7, 2001

Related U.S. Application Data
(60) Provisional application No. 60/263,231, filed on Jan. 23, 2001.

(30) Foreign Application Priority Data
Dec. 7, 2000 (FR) 00403440.1

(51) Int. Cl. C12Q 1/68; C07H 21/04; C12P 21/02; C12N 5/06; C07K 14/435

(52) U.S. Cl. 435/69.1; 435/320.1; 435/6; 435/325; 530/350; 536/23.5

(57) ABSTRACT

The present invention relates to nucleic acids corresponding to various exons of ABCA5, ABCA6, ABCA9, and ABCA10 genes as well as cDNAs encoding the novel full length of ABCA5, ABCA6, ABCA9, and ABCA10 proteins. The invention also relates to means for the detection of polymorphisms in general, and of mutations in particular, in the ABCA5, ABCA6, ABCA9, and ABCA10 genes or in the corresponding protein produced by the allelic form of the ABCA5, ABCA6, ABCA9, and ABCA10 genes.

Under the provisions of Section 119 of 35 U.S.C., his application claims priority to French application No. 00403440.1, filed Dec. 7, 2000. This application also claims the benefit of U.S. Provisional Application No. 60/263,231, filed Jan. 23, 2001, which is incorporated herein in its entirety.

The present invention relates to novel nucleic acids corresponding to the ABCA5, ABCA6, ABCA9, and ABCA10 genes, and cDNAs encoding novel ABCA5, ABCA6, ABCA9, and ABCA10 proteins. The invention also relates to means for the detection of polymorphisms in general, and mutations in particular in the ABCA5, ABCA6, ABCA9, and ABCA10 genes or corresponding proteins produced by the allelic forms of the ABCA5, ABCA6, ABCA9, and ABCA10 genes.

The genes of the ABC (ATP-binding cassette transporter) superfamily encode active transporter proteins, which are extremely well conserved during evolution, from bacteria to humans (Ames and Lecar, FASEB J., 1992, 6, 2660-2666). The ABC proteins are involved in extra- and intracellular membrane transport of various substrates, for example, ions, amino acids, peptides, sugars, vitamins, and steroid hormones. Among the 40 characterized humans members of the ABC superfamily, 11 members have been described as associated with human disease, such as, inter alia, ABCA1, ABCA4 (ABCR), and ABCC7 (CFTR), which are thought to be involved in Tangier disease (Bodzioch M et al., Nat. Genet., 1999, 22(4); 347-351; Brooks-Wilson et al., Nat Genet,1999, 22(4), 336-345; Rust S et al., Nat. Genet., 1999, 22, 352-355; Remaley A T et al., ), Stargardt disease (Lewis R A et al., Am. J. Hum. Genet., 1999, 64, 422-434), and cystic fibrosis (Riordan J M et al., Science, 1989, 245, 1066-1073), respectively. These associations reveal the importance of ABC gene family function. The discovery of new family gene members should provide insights into the physiopathology of additional human diseases.

The prototype ABC protein binds ATP and uses the energy from ATP hydrolysis to drive the transport of various molecules across cell membranes. The prototype protein contains two ATP-binding domains (nucleotide binding fold, NBF) and two transmembrane (TM) domains. The genes are typically organized as full transporters containing two of each domain, or half transporters with only one of each domain. Most full transporters are arranged in a TM-NBF-TM-NBF fashion (Dean et al., Curr Opin Genet, 1995, 5, 79-785).

Analysis of amino acids sequence alignments of the ATP-binding domains has allowed the ABC genes to be separated into sub-families (Allikmets et al., Hum Mol Genet, 1996, 5, 1649-1655). Currently, according to the recent HUGO classification, seven ABC gene sub-families named ABC (A to G) have been described in the human genome (ABC1, CFTR/MRP, MDR, ABC8, ALD, GCN20, OABP) with all except one (OABP) containing multiple members. For the most part, these sub-families contain genes that also display considerable conservation in the transmembrane domain sequences and have similar gene organization. However, ABC proteins transport very varied substrates, and some members of different sub-families have been shown to share more similarity in substrate recognition than do proteins within the same sub-family. Five of the sub-families also are represented in the yeast genome, indicating that these groups have been retained from an early time in the evolution of eukaryotes (Decottignies et al., Nat Genet, 1997, 137-45; Michaelis et al., 1995, Cold Spring Harbor Laboratory Press).

Several ABC transport proteins that have been identified in humans are associated with diseases. For example, cystic fibrosis is caused by mutations in the CFTR (cystic fibrosis transmembrane conductance regulator) gene (Riordan J M et al., Science, 1989, 245,1066-1073). Moreover, some multiple drug resistance phenotypes in tumor cells have been associated with the gene encoding the MDR (multi-drug resistance) protein, which also has an ABC transporter structure (Anticancer Drug Des. 1999 Apr14(2):115-31.). Other ABC transporters have been associated with neuronal and tumor conditions (U.S. Pat. No. 5,858,719) or potentially involved in diseases caused by impairment of the homeostasis of metals (Biochim. Biophys. Acta. 1999 Dec 6;1461(2):18-404. ). Likewise, another ABC transporter, designated PFIC2, appears to be involved in a progressive familial intrahepatic cholestasia form, this protein potentially being responsible, in humans, for the export of bile salts (Strautnieks S S et al, Nat Genet, 1998, 20, 233-238).

Among the ABC sub-families, the ABCA gene subfamily is probably the most evolutionarily complex. The ABCA genes and OABP are the only two sub-families of ABC genes that do not have identifiable orthologs in the yeast genome (Decottignies and Goffeau, 1997; Michaelis and Berkower, 1995). There is, however, at least one ABCA-related gene in C. elegans (ced-7) and several in Drosophila. Thus the ABCA genes appear to have diverged after eukaryotes became multicellular and developed more sophisticated transport requirements. To date, eleven members of the human ABCA sub-family have been described, making it the largest such group.

Full sequences of four genes of the ABCA sub-family have been described, revealing a complex exon-intron structure. The best characterized ABCA genes are ABCA4, and ABCA1. In mammals, the ABCA1 gene is highly expressed in macrophages and monocytes and is associated with the engulfment of apoptotic cells (Luciani et al, Genomics (1994) 21,150-9; Moynault et al., Biochem Soc Trans (1998) 26, 629-35; Wu et al., Cell (1998) 93, 951-60). The ced-7 gene, the ortholog of ABCA1 in C. elegans, also plays a role in the recognition and engulfment of apoptotic cells, suggesting a conserved function. Recently ABCA1 was demonstrated to be the gene responsible for Tangier disease, a disorder characterized by high levels of cholesterol in peripheral tissues and a very low level of HDLs, and for familial hypoalphalipoproteinemia (FHD) (Bodzioch et al., Nat Genet (1999) 22, 347-51; Brooks-Wilson et al., Nat Genet (1999) 336-45; Rust et al., Nat Genet (1999) 22, 352-5; Marcil et al., The Lancet (1999) 354,1341-46). The ABCA1 protein is proposed to function in the reverse transport of cholesterol from peripheral tissues via an interaction with the apolipoprotein 1 (ApoA-1) of HDL (Wang et al., J. Biol. Chem. (2000)).

The ABCA2 gene is highly expressed in the brain and ABCA3 in the lung, but no function has been ascribed to these loci. The ABCA4 gene is exclusively expressed in the rod photoreceptors of the retina, and mutations thereof are responsible for several pathologies of human eyes, such as retinal degenerative disorders (Allikmets et al., Science (1997) 277, 1805-1807; Allikmets et al., Nat Genet (1997) 15, 236-246; Sun et al., J Biol Chem (1999) 8269-81; Weng et al., Cell (1999) 98, 13-23; Cremers et al., Hum Mol Genet (1998) 7, 355-362; Martinez-Mir et al., Genomics (1997) 40, 142-146). ABCA4 is believed to transport retinal and/or retinal-phospholipid complexes from the rod photoreceptor outer segment disks to the cytoplasm, thereby facilitating phototransduction.

Characterization of new genes from the ABCA subfamily is likely to yield biologically important transporters that may have an translocase activity for membrane lipid transport and may play a major role in human pathologies.

Lipids are water-insoluble organic biomolecules, which are essential components with diverse biological functions, including the storage, transport, and metabolism of energy, and membrane structure and fluidity. Lipids are derived from two sources in humans and other animals: some lipids are ingested as dietary fats and oils and other lipids are biosynthesized by the human or animal. In mammals, at least 10% of the body weight is lipid, the bulk of which is in the form of triacylglycerols.

Triacylglycerols, also known as triglycerides and triacylglycerides, are made up of three fatty acids esterified to glycerol. Dietary triacylglycerols are stored in adipose tissues as a source of energy or hydrolyzed in the digestive tract by triacylglycerols lipases, the most important of which is pancreatic lipase. Triacylglycerols are transported between tissues in the form of lipoproteins.

Lipoproteins are micelle-like assemblies found in plasma and contain varying proportions of different types of lipids and proteins (called apoproteins). There are five main classes of plasma lipoproteins, the major function of which is lipid transport. These classes are, in order of increasing density, chylomicrons, very low density lipoproteins (VLDL), intermediate-density lipoproteins (IDL), low density lipoproteins (LDL), and high density lipoproteins (HDL). Although many types of lipids are found associated with each lipoprotein class, each class transports predominantly one type of lipid: triacylglycerols are transported in chylomicrons, VLDL, and IDL, while phospholipids and cholesterol esters are transported in HDL and LDL, respectively.

Phospholipids are di-fatty acid esters of glycerol phosphate, also containing a polar group coupled to the phosphate. Phospholipids are important structural components of cellular membranes. Phospholipids are hydrolyzed by enzymes called phospholipases. Phosphatidylcholine, an exemplary phospholipid, is a major component of most eukaryotic cell membranes.

Cholesterol is the metabolic precursor of steroid hormones and bile acids, as well as an essential constituent of cell membranes. In humans and other animals, cholesterol is ingested in the diet and also synthesized by the liver and other tissues. Cholesterol is transported between tissues in the form of cholesteryl esters in LDLs and other lipoproteins.

Membranes surround every living cell and serve as a barrier between the intracellular and extracellular compartments. Membranes also enclose the eukaryotic nucleus, make up the endoplasmic reticulum, and serve specialized functions such as in the myelin sheath that surrounds axons. A typical membrane contains about 40% lipid and 60% protein, but there is considerable variation. The major lipid components are phospholipids, specifically phosphatidylcholine and phosphatidylethanolamine, and cholesterol. The physicochemical properties of membranes, such as fluidity, can be changed by modification of either the fatty acid profiles of the phospholipids or the cholesterol content. Modulating the composition and organization of membrane lipids also modulates membrane-dependent cellular functions, such as receptor activity, endocytosis, and cholesterol flux.

High-density lipoproteins (HDL) are one of the five major classes of lipoproteins circulating in blood plasma. These lipoproteins are involved in various metabolic pathways such as lipid transport, the formation of bile acids, steroidogenesis, cell proliferation, and, in addition, interfere with the plasma proteinase systems.

HDLs are perfect free cholesterol acceptors and, in combination with enzymatic activities such as that of the cholesterol ester transfer protein (CETP), the lipoprotein lipase (LPL), the hepatic lipase (HL), and the lecithin:cholesterol acyltransferase (LCAT), play a major role in the reverse transport of cholesterol, i.e., the transport of excess cholesterol in peripheral cells to the liver for its elimination from the body in the form of bile acid. It has been demonstrated that the HDLs play a central role in the transport of cholesterol from peripheral tissues to the liver.

Various diseases linked to HDL deficiency have been described, including Tangiers disease, FHD disease, and LCAT deficiency. In addition, HDL-cholesterol deficiencies have been observed in patients suffering from malaria and diabetes (Kittl et al., 1992. Wein Klin Wochenschr 104 :21-4; Nilsson et al., 1990, J. Intern. Med., 227:151-5; Djoumessi, 1989, Pathol Biol., 37:909-11; Mohanty et al., 1992. Ann Trop Med Parasitol., 86 :601-6; Maurois et al., 1985, Biochimie, 67 :227-39; Grellier et al., 1997. Vox Sang. 72 :211-20; Agbedana et al., 1990, Ann Trop Med Parasitol., 84 :529-30; Erel et al., 1998, Haematologia, Budap, 29 :207-12; Cuisinier et al., 1990, Med Trop, 50 :91-5; Chander et al., 1998, Indian J Exp Biol., 36 :371-4; Efthimiou et al., 1992, Wein Klin Wochenschr., 104 :705-6; Baptista et al., 1996. Parasite, 3:335-40; Davis et al., 1993, J. Infect. 26 :279-85; Davis et al., 1995, J. Infect. 31:181-8; Pirich et al., 1993, Semin Thromb Hemost., 19:138-43; Tomlinson and Raper, 1996, Nat. Biotechnol., 14:717-21; Hager and Hajduk, 1997, Nature 385:823-6; Kwiterovich, 1995, Ann NY Acad Sci., 748 :313-30 ; Syvanne et al. 1995, Circulation, 92:364-70; and Syvanne et al., 1995, J. Lipid Res., 36:573-82). The deficiency involved in Tangier and/or FHD disease is linked to a cellular defect in the translocation of cellular cholesterol that causes a degradation of HDLs and leads to a disruption in lipoprotein metabolism.

Atherosclerosis is defined in histological terms by deposits (lipid or fibrolipid plaques) of lipids and of other blood derivatives in blood vessel walls, especially the large arteries (aorta, coronary arteries, carotid). These plaques, which are more or less calcified according to the degree of progression of the atherosclerosis process, may be coupled with lesions and are associated with the accumulation in the vessels of fatty deposits consisting essentially of cholesterol esters. Development of these plaques is accompanied by a thickening of the vessel wall, hypertrophy of the smooth muscle, appearance of foam cells (lipid-laden cells resulting from uncontrolled uptake of cholesterol by recruited macrophages), and accumulation of fibrous tissue. The atheromatous plaque protrudes markedly from the wall, endowing it with a stenosing character responsible for vascular occlusions by atheroma, thrombosis, or embolism, which occur in those patients who are most severely affected. These lesions can lead to serious cardiovascular pathologies, such as myocardial infarction, sudden death, cardiac insufficiency, and stroke.

Mutations within genes that play a role in lipoprotein metabolism have been identified. Specifically, several mutations in the apolipoprotein apoA-I gene have been characterized. These mutations are rare and may lead to a lack of production of apoA-I. Mutations in the genes encoding LPL or its activator apoC-II are associated with severe hypertriglyceridemias and substantially reduced HDL-C levels. Mutations in the gene encoding the enzyme LCAT also are associated with severe HDL deficiency.

In addition, dysfunctions in the reverse transport of cholesterol may be induced by physiological deficiencies affecting one or more of the steps in the transport of stored cholesterol, from the intracellular vesicles to the membrane surface where it is accepted by the HDLs.

Therefore, a need exists to identify genes involved in any of the steps in the metabolism of cholesterol and/or lipoproteins, and, in particular, genes associated with dysfunctions in the reverse transport of cholesterol from peripheral cells to the liver.

Applicants have discovered and characterized a gene cluster containing 4 new genes belonging to the ABCA protein sub-family, which have been designated ABCA5, ABCA6, ABCA9, and ABCA10. These new genes appear to be closely related to other ABCA subfamily members such as ABCA1 and ABCA8, particularly in the ATP-binding domain and in the C-terminal ATP binding domains. The newly discovered genes also show considerable conservation of amino acid sequence, particularly within the transmembrane region (TM) and the ATP-binding regions (NBD), and have a similar gene organization.

Surprisingly, Applicants have found these genes to be organized in a single large cluster on chromosome 17q24, in a head-to-tail fashion, with a similar intron/exon organization, suggesting that they have arisen from tandem duplication and that they may form a distinct functional group with the ABCA subfamily.

Furthermore, each of the newly discovered genes is transcribed with a tissue-specific distribution and presents a heterogenous pattern of expression, suggesting a regional and probably functional specialization of the corresponding proteins.

### SUMMARY OF THE INVENTION

The present invention relates to nucleic acids corresponding to the various human ABCA5, ABCA6, ABCA9, and ABCA10 genes, which are likely to be involved in the reverse transport of cholesterol, as well as in the membrane transport of lipophilic molecules, in particular, inflammation-mediating substances such as prostaglandins and prostacyclins, or in any pathology whose candidate chromosomal region is situated on chromosome 17, more precisely on the 17q arm and, still more precisely, in the 17q24 locus.

Thus, a first subject of the invention is a nucleic acid comprising a nucleotide sequence of any one of SEQ ID NOs: 1-4, and 9-126, or a complementary nucleotide sequence thereof.

The invention also relates to a nucleic acid comprising at least 8 consecutive nucleotides of a nucleotide sequence of any one of SEQ ID NOs: 1-4, and 9-126 or a complementary nucleotide sequence thereof.

The invention also relates to a nucleic acid having at least 80% nucleotide identity with a nucleic acid comprising a nucleotide sequence of any one of SEQ ID NOs: 1-4, and 9-126 or a complementary nucleotide sequence thereof.

The invention also relates to a nucleic acid having at least 85%, at least 90%, at least 95%, or at least 98% nucleotide identity with a nucleic acid comprising a nucleotide sequence of any one of SEQ ID NOs: 1-4 and 9-126 or a complementary nucleotide sequence thereof.

The invention also relates to a nucleic acid hybridizing, under high stringency conditions, with a nucleotide sequence of any one of SEQ ID NOs: 1-4 and 9-126 or a complementary nucleotide sequence thereof.

The invention also relates to nucleic acids, particularly cDNA molecules, which encode the full length human ABCA5, ABCA6, ABCA9, or ABCA10 proteins. Thus, the invention relates to a nucleic acid comprising a nucleotide sequence of any one of SEQ ID NOS: 1-4 or of a complementary nucleotide sequence.

The invention also relates to a nucleic acid comprising a nucleotide sequence as depicted in SEQ ID NOS: 1-4 or a complementary nucleotide sequence.

According to the invention, a nucleic acid comprising a nucleotide sequence of SEQ ID NO: 1 encodes a full length ABCA5 polypeptide of 1642 amino acids comprising the amino acid sequence of SEQ ID NO: 5.

According to the invention, a nucleic acid comprising a nucleotide sequence of SEQ ID NO: 2 encodes a full length ABCA6 polypeptide of 1617 amino acids comprising the amino acid sequence of SEQ ID NO: 6.

According to the invention, a nucleic acid comprising a nucleotide sequence of SEQ ID NO: 3 encodes a full length ABCA9 polypeptide of 1624 amino acids comprising the amino acid sequence of SEQ ID NO: 7.

According to the invention, a nucleic acid comprising a nucleotide sequence of SEQ ID NO: 4 encodes a full length ABCA10 polypeptide of 1543 amino acids comprising the amino acid sequence of SEQ ID NO: 8.

Thus, the invention also relates to a nucleic acid encoding a polypeptide comprising an amino acid sequence of any one of SEQ ID NOS: 5-8.

Thus, the invention also relates to a polypeptide comprising an amino acid sequence of any one of SEQ ID NOS: 5-8.

The invention also relates to a polypeptide comprising an amino acid sequence as depicted in any one of SEQ ID NOS: 5-8.

The invention also relates to a means for detecting polymorphisms in general, and mutations in particular, in the ABCA5, ABCA6, ABCA9, and ABCA10 genes or in the corresponding proteins produced by the allelic form of these genes.

According to another aspect, the invention relates to the nucleotide sequences of the ABCA5, ABCA6, ABCA9, and ABCA10 genes comprising at least one biallelic polymorphism such as, for example, a substitution, addition, or deletion of one or more nucleotides.

The invention also encompasses nucleotide probes and primers hybridizing with a nucleic acid sequence located in the region of any one of the ABCA5, ABCA6, ABCA9, and ABCA10 nucleic acids (genomic DNA, messenger RNA, cDNA), in particular, a nucleic acid sequence comprising any one of the mutations or polymorphisms.

The nucleotide probes or primers according to the invention comprise at least 8 consecutive nucleotides of a nucleic acid comprising any one of SEQ ID NOs: 1-4 and 9-126 or a complementary nucleotide sequence thereof. Nucleotide probes or primers according to the invention may have a length of 10, 12, 15, 18 or 20 to 25, 35, 40, 50, 70, 80, 100, 200, 500, 1000, 1500 consecutive nucleotides of a nucleic acid according to the invention, for example, a nucleic acid comprising any one of SEQ ID NOs: 1-4 and 9-126 or a complementary nucleotide sequence thereof.

Alternatively, a nucleotide probe or primer according to the invention will consist of and/or comprise fragments having a length of 12, 15, 18, 20, 25, 35, 40, 50, 100, 200, 500, 1000, 1500 consecutive nucleotides of a nucleic acid according to the invention, for example, a nucleic acid comprising any one of SEQ ID NOs: 1-4 and 9-126 or a complementary nucleotide sequence thereof.

The definition of a nucleotide probe or primer according to the invention, therefore, encompasses oligonucleotides that hybridize, under high stringency hybridization conditions defined below, with a nucleic acid comprising any one of SEQ ID NOs: 1-4 and 9-126 or a complementary nucleotide sequence thereof.

The probes and primers according to the invention may also comprise all or part of a nucleotide sequence comprising any one of SEQ ID NOs: 127-217 or a complementary nucleotide sequence thereof.

Nucleotide primers according to the invention may be used to amplify any one of the nucleic acids according to the invention, for example, a nucleic acid comprising a nucleotide sequence of any one of SEQ ID NOs: 1-4 and 9-126 or a complementary nucleotide sequence thereof.

According to the invention, some nucleotide primers specific for an ABCA5 gene, may be used to amplify a nucleic acid comprising SEQ ID NO: 1 and comprise a nucleotide sequence of any one of SEQ ID NOs: 127-150, or a complementary nucleotide sequence thereof.

The invention also relates to nucleotide primers that are specific for an ABCA6 gene, which may be used to amplify a nucleic acid comprising any one of SEQ ID NOs: 2 and 9-47 and comprise a nucleotide sequence of any one of SEQ ID NOs: 151-177 or a complementary nucleotide sequence.

The invention is further directed to nucleotide primers specific for an ABCA9 gene, which may be used to amplify a nucleic acid comprising any one of SEQ ID NOs: 3, and 48-86 and comprise a nucleotide sequence of any one of SEQ ID NOs: 178-209 or a complementary nucleotide sequence.

The present invention is further directed to nucleotide primers specific for an ABCA10 gene, which may be used to amplify a nucleic acid comprising any one of SEQ ID NOs: 4, and 87-126 and comprise a nucleotide sequence of any one of SEQ ID NOs: 210-217 or a complementary nucleotide sequence.

Another subject of the invention relates to a method of amplifying a nucleic acid according to the invention, for example, a nucleic acid comprising a) any one of SEQ ID NOs: 1-4 and 9-126 or a complementary nucleotide sequence thereof, or b) as depicted in any one of SEQ ID NOs: 1-4 and 9-126 or a complementary nucleotide sequence thereof, contained in a sample, said method comprising:

a) bringing the sample in which the presence of the target nucleic acid is suspected into contact with a pair of nucleotide primers whose hybridization position is located, respectively, on the 5′ side and on the 3′ side of the region of the target nucleic acid whose amplification is sought, in the presence of the reagents necessary for the amplification reaction;

b) performing an amplification reaction; and, optionally,

c) detecting the amplified nucleic acids.

The present invention also relates to a method of detecting the presence of a nucleic acid comprising a nucleotide sequence of any one of SEQ ID NOs: 1-4 and 9-126, or a complementary nucleotide sequence, or a nucleic acid fragment or variant of any one of SEQ ID NOs: 1-4 and 9-126 , or a complementary nucleotide sequence in a sample, said method comprising:

1) bringing one or more nucleotide probes according to the invention into contact with the sample to be tested; and

2) detecting the complex that may have formed between the probe(s) and the nucleic acid present in the sample.

According to an embodiment of the method of detection according to the invention, the oligonucleotide probes are immobilized on a support.

According to another embodiment, the oligonucleotide probes comprise a detectable marker.

Another subject of the invention is a box or kit for amplifying all or part of a nucleic acid comprising a) any one of SEQ ID NOs: 1-4 and 9-126 or a complementary nucleotide sequence thereof or b) any of the sequences as depicted in any one of SEQ ID NOs: 1-4 and 9-126 or of a complementary nucleotide sequence thereof, said box or kit comprising:

1) a pair of nucleotide primers in accordance with the invention, whose hybridization position is located, respectively, on the 5′ side and on the 3′ side of the target nucleic acid whose amplification is sought; and, optionally,

2) reagents necessary for an amplification reaction.

Such an amplification box or kit will preferably comprise at least one pair of nucleotide primers as described above.

The invention also relates to a box or kit for detecting the presence of a nucleic acid according to the invention in a sample, said box or kit comprising:

a) one or more nucleotide probes according to the invention;

b) appropriate reagents necessary for a hybridisation reaction.

According to one aspect, the detection box or kit is characterized in that the nucleotide probe(s) and primer(s)are immobilized on a support.

According to another aspect, the detection box or kit is characterized in that the nucleotide probe(s) and primer(s) comprise a detectable marker.

According to an embodiment of the detection kit described above, such a kit will comprise a plurality of oligonucleotide probes and/or primers in accordance with the invention that may be used to detect target nucleic acids of interest or, alternatively, to detect mutations in the coding and/or the non-coding regions of the nucleic acids according to the invention. According to another embodiment of the invention, the target nucleic acid comprises a nucleotide sequence of any one of SEQ ID NOs: 1-4 and 9-126 or of a complementary nucleic acid sequence. Alternatively, the target nucleic acid is a nucleic acid fragment or variant of a nucleic acid comprising any one of SEQ ID NOs: 1-4 and 9-126 or of a complementary nucleotide sequence.

According to another embodiment, a primer according to the invention comprises all or part of any one of SEQ ID NOs: 1-4, and 9-217 or a complementary sequence.

The invention also relates to a recombinant vector comprising a nucleic acid according to the invention. Such a recombinant vector may comprise a nucleic acid selected from:

a) a nucleic acid comprising a nucleotide sequence of any one of SEQ ID NOs: 1-4 and 9-126 or a complementary nucleotide sequence thereof,

b) a nucleic acid comprising a nucleotide sequence as depicted in any one of SEQ ID NOs: 1-4 and 9-126,or a complementary nucleotide sequence thereof,

c) a nucleic acid having at least eight consecutive nucleotides of a nucleic acid comprising a nucleotide sequence of any one of SEQ ID NOs: 1-4 and 9-126 or a complementary nucleotide sequence thereof;

d) a nucleic acid having at least 80% nucleotide identity with a nucleic acid comprising a nucleotide sequence of any one of SEQ ID NOs: 1-4 and 9-126 or a complementary nucleotide sequence thereof;

e) a nucleic acid having at least 85%, at least 90%, at least 95%, or at least 98% nucleotide identity with a nucleic acid comprising a nucleotide sequence of any one of SEQ ID NOs: 1-4 and 9-126 or a complementary nucleotide sequence thereof;

f) a nucleic acid hybridizing, under high stringency hybridization conditions, with a nucleic acid comprising a nucleotide sequence of any one of SEQ ID NOs: 1-4 and 9-126 or a complementary nucleotide sequence; and

g) a nucleic acid encoding a polypeptide comprising an amino acid sequence of any one of SEQ ID NOS: 5-8.

According to one embodiment, a recombinant vector according to the invention is used to amplify a nucleic acid inserted therein, following transformation or transfection of a desired cellular host.

According to another embodiment, a recombinant vector according to the invention is an expression vector comprising, in addition to a nucleic acid in accordance with the invention, a regulatory signal or nucleotide sequence that directs or controls transcription and/or translation of the nucleic acid and its encoded mRNA.

According to yet another embodiment, a recombinant vector according to the invention may comprise, for example, the following components:

(1) an element or signal for regulating the expression of the nucleic acid to be inserted, such as a promoter and/or enhancer sequence;

(2) a nucleotide coding region comprised within a nucleic acid according to the invention to be inserted into such a vector, said coding region being placed in phase with the regulatory element or signal described in (1); and

(3) an appropriate nucleic acid for initiation and termination of transcription of the nucleotide coding region of the nucleic acid described in (2).

The present invention also relates to a defective recombinant virus comprising a cDNA encoding any one of the ABCA5, ABCA6, ABCA9, and ABCA10 polypeptides involved in the transport of lipophilic substances, for example, mediators of inflammation, or in any pathology whose candidate chromosomal region is situated on chromosome 17, more precisely on the 17q arm, and, still more precisely, in the 17q24 locus.

In another embodiment of the invention, the defective recombinant virus comprises a genomic DNA (gDNA) encoding any one of the ABCA5, ABCA6, ABCA9, and ABCA10 polypeptides involved in the transport of lipophilic substances, inflammatory lipophilic substances. The ABCA5, ABCA6, ABCA9, and ABCA10 polypeptides may comprise amino acid sequences selected from SEQ ID NOS: 5-8, respectively.

In another embodiment, the invention relates to a defective recombinant virus comprising a nucleic acid encoding any one of the ABCA5, ABCA6, ABCA9, and ABCA10 polypeptides involved in the transport of inflammatory lipophilic substances, under the control of a promoter chosen from (Rous sarcoma virus (RSV)-LTR or the cytomegalovirus (CMV) early promoter.

According to an embodiment of the invention, a method of introducing a nucleic acid according to the invention into a host cell, for example, a host cell obtained from a mammal, in vivo, comprises a step during which a preparation comprising a pharmaceutically compatible vector and a “naked” nucleic acid according to the invention, placed under the control of appropriate regulatory sequences, is introduced by local injection at the site of the chosen tissue, for example, a smooth muscle tissue, the “naked” nucleic acid being absorbed by the cells of this tissue.

According to another embodiment of the invention, a composition is provided for the in vivo production of any one of the ABCA5, ABCA6, ABCA9, and ABCA10 proteins. This composition comprises a nucleic acid encoding the ABCA5, ABCA6, ABCA9, or ABCA10 polypeptide placed under the control of appropriate regulatory sequences in solution in a physiologically-acceptable vehicle and/or excipient.

Therefore, the present invention also relates to a composition comprising a nucleic acid encoding any one of the ABCA5, ABCA6, ABCA9, ABCA10 polypeptides, wherein the polypeptide comprises an amino acid sequence selected from SEQ ID NOS: 5-8, and wherein the nucleic acid is placed under the control of appropriate regulatory elements.

Consequently, the invention also relates to a pharmaceutical composition intended for the prevention of or treatment of a patient or subject affected by a dysfunction in the reverse transport of cholesterol or in the transport of inflammatory lipophilic substances, wherein the composition comprises a nucleic acid encoding any one of ABCA5, ABCA6, ABCA9, and ABCA10 proteins, in combination with one or more physiologically compatible excipients.

Such a composition may comprise a nucleic acid comprising a nucleotide sequence of any one of SEQ ID NOs: 1-4 and 9-126, wherein the nucleic acid is placed under the control of an appropriate regulatory element or signal.

In addition, the present invention is directed to a pharmaceutical composition intended for the prevention of or treatment of a patient or a subject affected by a dysfunction in the reverse transport of cholesterol or in the transport of liphophilic substances mediating inflammation, comprising a recombinant vector according to the invention in combination with one or more physiologically-compatible excipients.

The invention also relates to the use of a nucleic acid according to the invention encoding any one of ABCA5, ABCA6, ABCA9, and ABCA10 proteins for the manufacture of a medicament intended for the prevention of atherosclerosis, for example, for the treatment of subjects affected by a dysfunction of cholesterol reverse transport or transport of liphophilic substances mediating inflammation.

The invention also relates to the use of a recombinant vector according to the invention comprising a nucleic acid encoding any one of the ABCA5, ABCA6, ABCA9, and ABCA10 proteins for the manufacture of a medicament intended for the prevention of atherosclerosis in various forms or, for example, for the treatment of subjects affected by a dysfunction of cholesterol reverse transport or transport of liphophilic substances mediating inflammation.

The subject of the invention is therefore also a recombinant vector comprising a nucleic acid according to the invention that encodes any one of the ABCA5, ABCA6, ABCA9 and ABCA10 proteins or polypeptides involved in the metabolism of cholesterol or transport of liphophilic substances mediating inflammation.

The invention also relates to the use of such a recombinant vector for the preparation of a pharmaceutical composition intended for the treatment and/or for the prevention of diseases or conditions associated with deficiency of lipophilic substances signaling inflammation, or deficiency of the reverse transport of cholesterol, or deficiency of the transport of inflammatory lipophilic substances.

The present invention also relates to the use of cells genetically modified ex vivo with a recombinant vector according to the invention or to cells producing a recombinant vector, wherein the cells may be implanted in the body, to allow a prolonged and effective expression in vivo of any one of biologically active ABCA5, ABCA6, ABCA9, or ABCA10 polypeptide.

The invention also relates to the use of a nucleic acid according to the invention encoding any one of the ABCA5, ABCA6, ABCA9 and ABCA10 proteins for the manufacture of a medicament intended for the prevention and/or the treatment of subjects affected by a dysfunction of cholesterol reverse transport or inflammatory lipophilic substances transport.

The invention also relates to the use of a recombinant vector according to the invention comprising a nucleic acid encoding any one of the ABCA5, ABCA6, ABCA9 and ABCA10 polypeptides according to the invention for the manufacture of a medicament intended for the prevention and/or the treatment of subjects affected by a dysfunction of the reverse transport of cholesterol or inflammatory lipophilic substances transport.

The invention also relates to the use of a recombinant host cell according to the invention, comprising a nucleic acid encoding any one of the ABCA5, ABCA6, ABCA9 and ABCA10 polypeptides according to the invention for the manufacture of a medicament intended for the prevention and/or the treatment of subjects affected by a dysfunction of cholesterol reverse transport.

The invention also relates to the use of a recombinant vector according to the invention, for example, a defective recombinant virus, for the preparation of a pharmaceutical composition for the treatment and/or prevention of pathologies linked to the dysfunction of cholesterol reverse transport or inflammatory lipophilic substances transport.

The invention relates to the use of such a recombinant vector or defective recombinant virus for the preparation of a pharmaceutical composition intended for the treatment and/or for the prevention of cardiovascular disease linked to a deficiency in the reverse transport of cholesterol. Thus, the present invention also relates to a pharmaceutical composition comprising one or more recombinant vectors or defective recombinant viruses according to the invention.

The present invention also relates to the use of cells genetically modified ex vivo with a virus according to the invention and to cells producing such viruses, which may be implanted in the body, allowing a prolonged and effective expression in vivo of any one of biologically active of ABCA5, ABCA6, ABCA9 or ABCA10 protein.

The present invention shows that it is possible to incorporate a nucleic acid encoding any one of the ABCA5, ABCA6, ABCA9, and ABCA10 polypeptides according to the invention into a viral vector, and that these vectors make it possible to express a biologically active, mature polypeptide. Moreover, the invention shows that the in vivo expression of any one of the ABCA5, ABCA6, ABCA9, and ABCA10 proteins may be obtained by direct administration of an adenovirus or by implantation of a producing cell or of a cell genetically modified by an adenovirus or by a retrovirus incorporating such a nucleic acid.

In this regard, another subject of the invention is any mammalian cell infected with one or more defective recombinant viruses according to the invention. The invention also encompases any population of human cells infected with these viruses. These may be, for example, of blood origin (totipotent stem cells or precursors), fibroblasts, myoblasts, hepatocytes, keratinocytes, smooth muscle, endothelial cells, glial cells, and the like.

Another subject of the invention is an implant comprising mammalian cells infected with one or more defective recombinant viruses according to the invention or cells producing recombinant viruses and an extracellular matrix. In general, the implants according to the invention comprise 10⁵ to 10¹⁰ cells. In one embodiment, the implants comprise 10⁶ to 10⁸ cells.

In the implants of the invention, the extracellular matrix may additionally comprise a gelling compound and, optionally, a support for the anchorage of the cells.

The invention also relates to a recombinant host cell comprising a nucleic acid of the invention, for example, a nucleic acid comprising any one of SEQ ID NOS: 1-4 and 9-126 or of a complementary nucleotide sequence.

The invention also relates to a recombinant host cell comprising a nucleic acid of the invention, for example, a nucleic acid comprising a nucleotide sequence as depicted in any one SEQ ID NOS: 1-4 and 9-126 or of a complementary nucleotide sequence.

According to another aspect, the invention encompasses a recombinant host cell comprising a recombinant vector according to the invention. Therefore, the invention also relates to a recombinant host cell comprising a recombinant vector comprising any of the nucleic acids of the invention, for example, a nucleic acid comprising any one nucleotide sequence of SEQ ID NOS: 1-4 and 9-126 or a complementary nucleotide sequence thereof.

The invention also relates to a recombinant host cell comprising a recombinant vector comprising a nucleic acid comprising any one of SEQ ID NOs: 1-4 and 9-126 or a complementary nucleotide sequence thereof.

The invention also relates to a recombinant host cell comprising a recombinant vector comprising a nucleic acid comprising a nucleotide sequence as depicted in any one of SEQ ID NOs:1-4 and 9-126 or of a complementary nucleotide sequence thereof.

The invention also relates to a recombinant host cell comprising a recombinant vector comprising a nucleic acid encoding a polypeptide comprising any one amino acid sequence of SEQ ID NOs: 5-8.

The invention also relates to a method for the production of a polypeptide comprising an amino acid sequence of any one of SEQ ID NOs: 1-4 and 9-126 or of a peptide fragment or a variant thereof, said method comprising:

a) inserting a nucleic acid encoding said polypeptide into an appropriate vector;

b) culturing, in an appropriate culture medium under conditions that allow the expression of said polypeptide, a previously transformed host cell or transfecting a host cell with the recombinant vector of step a);

c) recovering the conditioned culture medium or lysing the host cell, for example, by sonication or by osmotic shock;

d) separating and purifying said polypeptide from said culture medium or, alternatively, from the cell lysates obtained in step c); and

e) where appropriate, characterizing the recombinant polypeptide produced.

A polypeptide termed “homologous” to a polypeptide having an amino acid sequence selected from SEQ ID NOS: 5-8 also is part of the invention. Such a homologous polypeptide comprises an amino acid sequence possessing one or more substitutions of an amino acid by an equivalent amino acid.

The ABCA5, ABCA6, ABCA9, ABCA10 polypeptides according to the invention, including 1) a polypeptide comprising an amino acid sequence of any one of SEQ ID NOs: 5-8, 2) a polypeptide fragment or variant of a polypeptide comprising an amino acid sequence of any one of SEQ ID NOs: 5-8, and 3) a polypeptide termed “homologous” to a polypeptide comprising amino acid sequence selected from SEQ ID NOs: 5-8.

In one embodiment, an antibody according to the invention is directed against 1) a polypeptide comprising an amino acid sequence of any one of SEQ ID NOs: 5-8, 2) a polypeptide fragment or variant of a polypeptide comprising an amino acid sequence selected from SEQ ID NOs: 5-8, or 3) a polypeptide termed “homologous” to a polypeptide comprising amino acid sequence selected from SEQ ID NOS: 5-8. Such an antibody may be produced by any known techniques, including the trioma technique or the hybridoma technique described by Kozbor et al. (Immunology Today, (1983) 4:72).

Thus, the subject of the invention is, in addition, a method of detecting the presence of any one of the polypeptides according to the invention in a sample, said method comprising:

a) bringing the sample to be tested into contact with an antibody directed against 1) a polypeptide comprising an amino acid sequence of any one of SEQ ID NOs: 5-8, 2) a polypeptide fragment or variant of a polypeptide comprising an amino acid sequence selected from SEQ ID NOs: 5-8, or 3) a polypeptide termed “homologous” to a polypeptide comprising amino acid sequence of any one of SEQ ID NOS: 5-8; and

b) detecting the antigen/antibody complex formed.

The invention also relates to a box or kit for diagnosis or for detecting the presence of any one of polypeptide in accordance with the invention in a sample, said box comprising:

a) an antibody directed against 1) a polypeptide comprising an amino acid sequence of any one of SEQ ID NOs: 5-8, 2) a polypeptide fragment or variant of a polypeptide comprising an amino acid sequence of any one of SEQ ID NOs: 5-8, or 3) a polypeptide “homologous” to a polypeptide comprising amino acid sequence of SEQ ID NOS: 5-8; and

b) a reagent allowing the detection of the antigen/antibody complexes formed.

The invention also relates to a pharmaceutical composition comprising a nucleic acid according to the invention.

The invention also provides compositions comprising a nucleic acid encoding any one of the ABCA5, ABCA6, ABCA9, and ABCA10 polypeptides according to the invention and compositions comprising any one of the ABCA5, ABCA6, ABCA9, ABCA10 polypeptides according to the invention intended for the treatment of diseases linked to a deficiency of cholesterol reverse transport or inflammatory lipophilic substances transport.

The present invention also relates to a new therapeutic approach for the treatment of pathologies linked to a deficiency of cholesterol reverse transport or inflammatory lipophilic substances transport, comprising transferring and expressing in vivo nucleic acids encoding any one of the ABCA5, ABCA6, ABCA9, and ABCA10 proteins according to the invention.

Thus, the present invention offers a new approach to the treatment and/or prevention of pathologies linked to the abnormalities of cholesterol reverse transport or inflammatory lipophilic substances. Specifically, the present invention provides methods to restore or promote improved cholesterol reverse transport or improved inflammatory lipophilic substances transport in a patient or subject.

Consequently, the invention also relates to a composition intended for the prevention and/or treatment of subjects affected by a dysfunction of cholesterol reverse transport or inflammatory lipophilic substances transport, comprising a nucleic acid encoding any one of the ABCA5, ABCA6, ABCA9, and ABCA10 proteins in combination with one or more physiologically-compatible vehicle and/or excipient.

According to one embodiment of the invention, a composition is provided for the in vivo production of any one of the ABCA5, ABCA6, ABCA9, and ABCA10 proteins. This composition comprises a nucleic acid encoding any one of the ABCA5, ABCA6, ABCA9, and ABCA10 polypeptides placed under the control of appropriate regulatory sequences in solution in a physiologically-compatible vehicle and/or excipient.

Therefore, the present invention also relates to a composition comprising a nucleic acid encoding a polypeptide comprising an amino acid sequence of any one of SEQ ID NOS: 5-8, wherein the nucleic acid is placed under the control of appropriate regulatory elements.

Such a composition may comprise a nucleic acid comprising a nucleotide sequence of any one of SEQ ID NOS: 1-4 and 9-126, placed under the control of appropriate regulatory elements.

The invention also relates to a composition intended for the prevention of or treatment of subjects affected by a dysfunction of cholesterol reverse transport or inflammatory lipophilic substances transport, comprising a recombinant vector according to the invention in combination with one or more physiologically-compatible vehicle and/or excipient.

According to another aspect, the subject of the invention is also a preventive or curative therapeutic method of treating diseases caused by a deficiency of cholesterol reverse transport or inflammatory lipophilic substances transport, such a method comprising administering to a patient a nucleic acid encoding any one of the ABCA5, ABCA6, ABCA9, and ABCA10 polypeptides according to the invention, said nucleic acid being combined with one or more physiologically-appropriate vehicles and/or excipients.

The invention relates to a composition for the prevention and/or treatment of a patient or subject affected by a dysfunction of cholesterol reverse transport or inflammatory lipophilic substances transport, comprising a therapeutically effective quantity of a polypeptide having an amino acid sequence selected from SEQ ID NOS: 5-8 combined with one or more physiologically-appropriate vehicles and/or excipients.

According to one embodiment, a method of introducing at least a nucleic acid according to the invention into a host cell, for example, a host cell obtained from a mammal, in vivo, comprises a step during which a preparation comprising a pharmaceutically compatible vector and a “naked” nucleic acid according to the invention, placed under the control of appropriate regulatory sequences, is introduced by local injection at the site of the chosen tissue, for example, a smooth muscle tissue, the “naked” nucleic acid being absorbed by the cells of this tissue.

According to yet another aspect, the subject of the invention is also a preventive or curative therapeutic method of treating diseases caused by a deficiency of cholesterol reverse transport or inflammatory lipophilic substances transport, such a method comprising administering to a patient a therapeutically effective quantity of at least one the ABCA5, ABCA6, ABCA9, or ABCA10 polypeptides according to the invention, said polypeptide being combined with one or more physiologically-appropriate vehicles and/or excipients.

The invention also provides methods for screening small molecules and compounds that act on any one of the ABCA5, ABCA6, ABCA9, and ABCA10 proteins to identify agonists and antagonists of such polypeptides that can restore or promote improved cholesterol reverse transport or inflammatory lipophilic substances transport to effectively cure and or prevent dysfunctions thereof. These methods are useful for identifying small molecules and compounds for therapeutic use in the treatment of diseases due to a deficiency of cholesterol reverse transport or inflammatory lipophilic substances transport.

The invention also relates to the use of any one of the ABCA5, ABCA6, ABCA9, and ABCA10 polypeptides or a cell expressing any one of the ABCA5, ABCA6, ABCA9, and ABCA10 polypeptides according to the invention for screening active ingredients for the prevention and/or treatment of diseases resulting from a dysfunction cholesterol reverse transport or inflammatory lipophilic substances transport.

The invention also relates to a method of screening a compound or small molecule, an agonist or antagonist of any one of the ABCA5, ABCA6, ABCA9, and ABCA10 polypeptides, said method comprising:

a) preparing a membrane vesicle comprising any one of the ABCA5, ABCA6, ABCA9, and ABCA10 polypeptides and a lipid substrate comprising a detectable marker;

b) incubating the vesicle obtained in step a) with an agonist or antagonist candidate compound;

c) qualitatively and/or quantitatively measuring release of the lipid substrate comprising a detectable marker; and

d) comparing the release measurement obtained in step c) with a measurement of release of a labelled lipid substrate by a vesicle that has not been previously incubated with the agonist or antagonist candidate compound.

In a one embodiment of this method, the ABCA5,ABCA6,ABCA9, and ABCA10 polypeptides comprise SEQ ID NOS: 5-8, respectively.

The invention also relates to a method of screening a compound or small molecule, an agonist or antagonist of any one of ABCA5, ABCA6, ABCA9, and ABCA10 polypeptides, said method comprising:

a) obtaining a cell, for example, a cell line, that, either naturally or after transfecting the cell with any one of the ABCA5, ABCA6, ABCA9, and ABCA10 encoding nucleic acids, expresses the ABCA5, ABCA6, ABCA9, or ABCA10 polypeptide;

b) incubating the cell of step a) in the presence of an anion labelled with a detectable marker;

c) washing the cell of step b) in order to remove the excess of the labelled anion which has not penetrated into these cells;

d) incubating the cell obtained in step c) with an agonist or antagonist candidate compound for the any one of the ABCA5, ABCA6, ABCA9, and ABCA10 polypeptides;

e) measuring efflux of the labelled anion; and

f) comparing the value of efflux of the labelled anion determined in step e) with the value of efflux of a labelled anion measured with a cell that has not been previously incubated in the presence of the agonist or antagonist candidate compound for any one of the ABCA5, ABCA6, ABCA9, and ABCA10 polypeptides.

In one embodiment of this method, the ABCA5, ABCA6, ABCA9, and ABCA10 polypeptides comprise SEQ ID NOS: 5-8, respectively.

The invention also relates to a method of screening a compound or small molecule, an agonist or antagonist of any one of the ABCA5, ABCA6, ABCA9, and ABCA10 polypeptides, said method comprising:

a) culturing cells of a human monocytic line in an appropriate culture medium, in the presence of purified human albumin;

b) incubating the cells of step a) simultaneously in the presence of a compound stimulating the production of IL-1 beta and of the agonist or antagonist candidate compound;

c) incubating the cells obtained in step b) in the presence of an appropriate concentration of ATP;

d) measuring IL-1 beta released into the cell culture supernatant; and

e) comparing the value of the release of the IL-1 beta obtained in step d) with the value of the IL-1 beta released into the culture supernatant of cells that have not been previously incubated in the presence of the agonist or antagonist candidate compound.

### BRIEF DESCRIPTION OF THE DRAWINGS

FIG. 1: represents the Map of the 17q24 region containing the ABCA5, 6, 9, and 10 genes. A physical map of the portion of chromosome 17q24 is shown containing the 5 ABCA genes. Location of the microsatellite marker D17S940 locus is indicated along with the boundaries of BAC clones hRPK.235_I_{—}10 and hRPK.293_K_{—}20 (GenBank accession #s AC005495, AC005922). Gene orientation is indicated by the arrows, and the size and location of the corresponding transcripts is shown below the map. 0 indicates the initiation codon; | represents the stop codon;—symbolizes the working draft sequences.

FIG. 2: represents the alignment of ABC1-like genes. An alignment of the amino acid sequence of the full-length ABCA6, 8, and 9 open reading frames and the partial sequence of ABCA5 is shown as aligned to ABCA1.

FIG. 3: Maximum parsimony tree of ABC1-like genes. Phylogenetic trees were constructed with the alignment of the N- and C-terminal ATP-binding domains' sequence by both neighbor joining and maximum parsimony methods.

FIG. 4: Northern blot analysis of poly(A)+ RNA from 20 human tissues: pancreas (lane 1), kidney (2), skeletal muscle (3), liver (4), lung (5), placenta (6), brain (7), heart (8), leukocyte (9), colon (10), small intestine (11), ovary (12), testis (13), prostate (14), thymus (15), spleen (16), fetal kidney (17), fetal liver (18), fetal lung (19) and fetal brain (20). Hybridization was with a probe specific for either ABCA5 (A), ABCA6 (B), ABCA9 (C), or ABCA10 (D).

FIG. 5: displays the pattern of expression of the gene encoding the ABCA9 protein by in situ hybridization using an antisense ABCA9 RNA probe on a normal renal artery section showing medial smooth muscle (60X).

FIG. 6: displays the pattern of expression of the gene encoding the ABCA9 protein by in situ hybridization using an antisense ABCA9 RNA probe on a normal renal artery section showing adventitial nerve and Schwann cells (60X).

FIG. 7: displays the pattern of expression of the gene encoding the ABCA9 protein by in situ hybridization using an antisense ABCA9 RNA probe on a normal renal artery section showing adjacent ganglions and Schwann cells (60X).

FIG. 8: displays the pattern of expression of the gene encoding the ABCA9 protein by in situ hybridization using an antisense ABCA9 RNA probe on an adjacent kidney section showing a collecting duct epithelium (60X).

FIG. 9: displays the pattern of expression of the gene encoding the ABCA9 protein by in situ hybridization using an antisense ABCA9 RNA probe on an adjacent kidney section showing a renal tubular epithelial (60X).

FIG. 10: displays the pattern of expression of the gene encoding the ABCA9 protein by in situ hybridization using an antisense ABCA9 RNA probe on a section of normal heart showing cardiac myocytes (60X).

FIG. 11: displays the pattern of expression of the gene encoding the ABCA9 protein by in situ hybridization using an antisense ABCA9 RNA probe on a section of normal heart showing interstitial vascular endothelial cells and fibroblasts (60X).

FIG. 12: displays the pattern of expression of the gene encoding the ABCA10 protein by in situ hybridization using an antisense ABCA10 RNA probe on a section of arterial tissues showing an adjacent lymph node, lymphocytes and macrophages (60X).

FIG. 13: displays the pattern of expression of the gene encoding the ABCA10 protein by in situ hybridization using an antisense ABCA10 RNA probe on a section of arterial tissues showing Schwann cells in a nerve (60X).

FIG. 14: displays the pattern of expression of the gene encoding the ABCA10 protein by in situ hybridization using an antisense ABCA10 RNA probe on a myocardial tissue section showing myointimal cells in an atheroma (60X).

FIG. 15: displays the pattern of expression of the gene encoding the ABCA10 protein by in situ hybridization using an antisense ABCA10 RNA probe on an tissue section adjacent to a myocardial tissue, showing a ganglion and Schwann cells (60X).

FIG. 16: displays the pattern of expression of the gene encoding the ABCA10 protein by in situ hybridization using an antisense ABCA10 RNA probe on a skeletal tissue section showing macrophages (60X).

FIG. 17: displays the pattern of expression of the gene encoding the ABCA10 protein by in situ hybridization using an antisense ABCA10 RNA probe on a skeletal tissue section showing Schwann cells in a nerve (60X).

### DETAILED DESCRIPTION OF THE INVENTION

General Definitions

The present invention encompasses the isolation of human genes encoding the ABCA5, ABCA6, ABCA9, and ABCA10 polypeptides of the invention, including full length or naturally occurring forms of ABCA5, ABCA6, ABCA9, and ABCA10 and any antigenic fragments thereof from any animal, including mammals, for example humans, and birds.

In accordance with the present invention, conventional molecular biology, microbiology, and recombinant DNA techniques within the skill of the art are used. Such techniques are fully explained in the literature (Sambrook et al., 1989. Molecular cloning a laboratory manual. 2ed. Cold Spring Harbor Laboratory, Cold Spring Harbor, N.Y.; Glover, 1985, DNA Cloning: A practical approach, volumes I and II oligonucleotide synthesis, MRL Press, LTD., Oxford, U.K.; Hames and Higgins, 1985, Transcription and translation; Hames and Higgins, 1984, Animal Cell Culture; Freshney, 1986, Immobilized Cells And Enzymes, IRL Press; and Perbal, 1984, A practical guide to molecular cloning).

As used herein, the term “gene” refers to an assembly of nucleotides that encode a polypeptide and includes cDNA and genomic DNA nucleic acids.

The term “isolated” for the purposes of the present invention refers to a biological material (nucleic acid or protein) that has been removed from its original environment (the environment in which it is naturally present). For example, a polynucleotide present in the natural state in a plant or in an animal is not isolated. The same polynucleotide separated from the adjacent nucleic acids in which it is naturally inserted in the genome of the plant or animal is considered as being “isolated”.

An “isolated” polynucleotide may be included in a vector and/or such a polynucleotide may be included in a composition and remain nevertheless in the isolated state because of the fact that the vector or the composition does not constitute its natural environment.

The term “purified” does not require the material to be present in a form exhibiting absolute purity exclusive of the presence of other compounds. It is a relative definition. A polynucleotide is in the “purified” state after purification from the starting material or from the natural material by at least one order of magnitude.

For the purposes of the present description, the expression “nucleotide sequence” is used to designate either a polynucleotide or a nucleic acid. The expression “nucleotide sequence” covers the genetic material itself and is therefore not restricted to the information relating to its sequence.

The terms “nucleic acid”, “polynucleotide”, “oligonucleotide” or “nucleotide sequence” encompass RNA, DNA, or cDNA sequences, and RNA/DNA hybrid sequences of more than one nucleotide, either in the single-stranded form or in the duplex, double-stranded form.

A “nucleic acid” is a polymeric compound comprised of covalently-linked subunits called nucleotides. The term “nucleic acid” includes polyribonucleic acid (RNA) and polydeoxyribonucleic acid (DNA), both of which may be single-stranded or double-stranded. DNA includes cDNA, genomic DNA, synthetic DNA, and semi-synthetic DNA. The sequence of nucleotides that encodes a protein is called the sense sequence or coding sequence.

The term “nucleotide” designates both the natural nucleotides (A, T, G, C) as well as modified nucleotides that comprise at least one modification such as (1) an analog of a purine, (2) an analog of a pyrimidine, or (3) an analogous sugar, examples of such modified nucleotides are described, for example, in the PCT application No. WO 95/04 064.

For the purposes of the present invention, a first polynucleotide is considered as being “complementary” to a second polynucleotide when each base of the first nucleotide is paired with the complementary base of the second polynucleotide whose orientation is reversed. The complementary bases are A and T (or A and U), or C and G.

“Heterologous” DNA refers to DNA not naturally located in the cell or in a chromosomal site of the cell. The heterologous DNA may include a gene foreign to the cell.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.) (Reeck et al., 1987, Cell 50 :667)). Such proteins (and their encoding genes) have sequence homology, as reflected by their high degree of sequence similarity.

Accordingly, the term “sequence similarity” in all its grammatical forms refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and not a common evolutionary origin.

For example, two DNA sequences are “substantially homologous” or “substantially similar” when at least about 50% (preferably at least about 75%, and more preferably at least about 90 or 95%) of the nucleotides match over the defined length of the DNA sequences. Sequences that are substantially homologous can be identified by comparing the sequences using standard software available in sequence data banks or in a Southern hybridization experiment under, for example, stringent conditions as defined for that particular system. Defining appropriate hybridization conditions is within the skill of the art. See, e.g., Maniatis et al., supra; Glover et al. (1985. DNA Cloning: A practical approach, volumes I and II oligonucleotide synthesis, MRL Press, Ltd, Oxford, U.K.); Hames and Higgins (1985. Transcription and Translation).

Similarly, two amino acid sequences are “substantially homologous” or “substantially similar” when greater than 30% of the amino acids are identical, or greater than about 60% are similar (functionally identical). Preferably, the similar or homologous sequences are identified by alignment using, for example, the GCG (Genetics Computer Group, Program Manual for the GCG Package, Version 7, Madison, Wis.) pileup program.

The “percentage identity” between two nucleotide or amino acid sequences, for the purposes of the present invention, may be determined by comparing two sequences aligned optimally through a window for comparison.

The portion of the nucleotide or polypeptide sequence in the window for comparison may thus comprise additions or deletions (for example “gaps”) relative to the reference sequence (which does not comprise these additions or these deletions) so as to obtain an optimum alignment of the two sequences.

The percentage identity is calculated by determining the number of positions at which an identical nucleic base or an identical amino acid residue is observed for the two sequences (nucleic or peptide) compared, dividing the number of positions at which there is identity between the two bases or amino acid residues by the total number of positions in the window for comparison, and then multiplying the result by 100 in order to obtain the percentage sequence identity.

The optimum sequence alignment for the comparison may be achieved using a computer with the aid of known algorithms contained in the package from the company Wisconsin Genetics Software Package, Genetics Computer Group (Gcg), 575 Science Doctor, Madison, Wis.

By way of illustration, it will be possible to produce the percentage sequence identity with the aid of the BLAST software (versions BLAST 1.4.9 of March 1996, BLAST 2.0.4 of February 1998 and BLAST 2.0.6 of September 1998), using exclusively the default parameters (Altschul et al, 1990, Mol. Biol., 215:403-410; Altschul et al, 1997, Nucleic Acids Res., 25:3389-3402). Blast searches for sequences similar/homologous to a reference “request” sequence, with the aid of the Altschul et al. algorithm. The request sequence and the databases used may be of the peptide or nucleic types, any combination being possible.

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity and not to the numbering of the amino acid residues or nucleotide bases.

A gene encoding any one of the ABCA5, ABCA6, ABCA9 and ABCA10 polypeptides of the invention, whether genomic DNA or cDNA, can be isolated from any source, for example, from a human cDNA or genomic library. Methods for obtaining genes are well known in the art as described above (see, e.g., Sambrook et al., 1989, Molecular cloning: a laboratory manual 2ed. Cold Spring Harbor Laboratory, Cold Spring Harbor, N.Y.).

Accordingly, any animal cell can serve as the nucleic acid source for the molecular cloning of any one of the ABCA5, ABCA6, ABCA9, and ABCA10 genes. The DNA may be obtained by standard procedures known in the art from cloned DNA (e.g., a DNA “library”) and preferably is obtained from a cDNA library prepared from tissues with high level expression of the protein and/or the transcripts, by chemical synthesis, by cDNA cloning, or by the cloning of genomic DNA, or fragments thereof, purified from the desired cell (See, for example, Sambrook et al., 1989, Molecular cloning: a laboratory manual. 2ed. Cold Spring Harbor Laboratory, Cold Spring Harbor, N.Y.; Glover, 1985, DNA Cloning: A Practical Approach, Volumes I and II Oligonucleotide Synthesis, MRL Press, Ltd., Oxford, U.K).

Clones derived from genomic DNA may contain regulatory and intron DNA regions in addition to coding regions; clones derived from cDNA will not contain intron sequences. Whatever the source, the gene should be molecularly cloned into a suitable vector for propagation of the gene.

In the molecular cloning of the gene from genomic DNA, DNA fragments are generated, some of which will encode the desired gene. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including, but not limited to, agarose and polyacrylamide gel electrophoresis and column chromatography.

Once the DNA fragments are generated, identification of the specific DNA fragment containing the desired ABCA5, ABCA, ABCA9, and ABCA10 genes may be accomplished in a number of ways. For example, if an amount of a portion of one of ABCA5, ABCA6, ABCA9, and ABCA10 genes or its specific RNA, or a fragment thereof, is available and can be purified and labelled, the generated DNA fragments may be screened by nucleic acid hybridization to the labelled probe (Benton and Davis, Science (1977), 196:180; Grunstein et al., Proc.Natl. Acad. Sci. U.S.A. (1975) 72:3961). For example, a set of oligonucleotides corresponding to the partial coding sequence information obtained for the ABCA5, ABCA6, ABCA9, ABCA10 proteins can be prepared and used as probes for DNA encoding any one of the ABCA5, ABCA6, ABCA9 and ABCA10 genes, as was done in a specific example, infra, or as primers for cDNA or mRNA synthesis (e.g., in combination with a poly-T primer for RT-PCR). Preferably, a fragment is selected that is highly unique to one of the ABCA5, ABCA6, ABCA9, and ABCA10 nucleic acids or polypeptides of the invention. Those DNA fragments with substantial homology to the probe will hybridize. As noted above, the greater the degree of homology, the more stringent hybridization conditions can be used. In one embodiment, various stringency hybridization conditions are used to identify homologous ABCA5, ABCA6, ABCA9, and ABCA10 genes.

Further selection can be carried out on the basis of the properties of the gene, e.g., if the gene encodes a protein product having the isoelectric, electrophoretic, amino acid composition, or partial amino acid sequence of one of the ABCA5, ABCA6, ABCA9, and ABCA10 proteins as disclosed herein. Thus, the presence of the gene may be detected by assays based on the physical, chemical, or immunological properties of its expressed product. For example, cDNA clones or DNA clones which hybrid-select the proper mRNAs can be selected which produce a protein that, e.g., has similar or identical electrophoretic migration, isoelectric focusing, or non-equilibrium pH gel electrophoresis behaviour, proteolytic digestion maps, or antigenic properties as known for ABCA5, ABCA6, ABCA9, and ABCA10.

The ABCA5, ABCA6, ABCA9, and ABCA10 genes of the invention may also be identified by mRNA selection, i.e., by nucleic acid hybridization followed by in vitro translation. According to this procedure, nucleotide fragments are used to isolate complementary mRNAs by hybridization. Such DNA fragments may represent available, purified ABCA5, ABCA6, ABCA9, ABCA10 DNAs or may be synthetic oligonucleotides designed from the partial coding sequence information. Immunoprecipitation analysis or functional assays (e.g., tyrosine phosphatase activity) of the in vitro translation products of the products of the isolated mRNAs identifies the mRNA and, therefore, the complementary DNA fragments that contain the desired sequences. In addition, specific mRNAs may be selected by adsorption of polysomes isolated from cells to immobilized antibodies specifically directed against any one of the ABCA5, ABCA6, ABCA9, and ABCA10 polypeptides of the invention.

Radiolabeled ABCA5, ABCA6, ABCA 9, and ABCA10 cDNAs can be synthesized using the selected mRNA (from the adsorbed polysomes) as a template. The radiolabeled mRNA or cDNA may then be used as a probe to identify homologous ABCA5, ABCA6, ABCA9, and ABCA10 DNA fragments from among other genomic DNA fragments.

“Variant” of a nucleic acid according to the invention will be understood to mean a nucleic acid that differs by one or more bases relative to the reference polynucleotide. A variant nucleic acid may be of natural origin, such as an allelic variant which exists naturally, or it may be a nonnatural variant obtained, for example, by mutagenic techniques.

In general, the differences between the reference (generally, wild-type) nucleic acid and the variant nucleic acid are small such that the nucleotide sequences of the reference nucleic acid and of the variant nucleic acid are very similar and, in many regions, identical. The nucleotide modifications present in a variant nucleic acid may be silent, which means that they do not alter the amino acid sequences encoded by said variant nucleic acid. However, the changes in nucleotides in a variant nucleic acid may also result in substitutions, additions, or deletions in the polypeptide encoded by the variant nucleic acid in relation to the polypeptides encoded by the reference nucleic acid. In addition, nucleotide modifications in the coding regions may produce conservative or non-conservative substitutions in the amino acid sequence of the polypeptide.

Preferably, the variant nucleic acids according to the invention encode polypeptides that substantially conserve the same function or biological activity as the polypeptide of the reference nucleic acid or, alternatively, the capacity to be recognized by antibodies directed against the polypeptides encoded by the initial reference nucleic acid. Some variant nucleic acids will thus encode mutated forms of the polypeptides whose systematic study will make it possible to deduce structure-activity relationships of the proteins in question. Knowledge of these variants in relation to the disease studied is essential since it makes it possible to understand the molecular cause of the pathology.

“Fragment” will be understood to mean a nucleotide sequence of reduced length relative to the reference nucleic acid and comprising, over the common portion, a nucleotide sequence identical to the reference nucleic acid. Such a nucleic acid “fragment” according to the invention may be, where appropriate, included in a larger polynucleotide of which it is a constituent. Such fragments comprise or, alternatively, consist of, oligonucleotides ranging in length from 8, 10, 12, 15, 18, 20 to 25, 30, 40, 50, 70, 80, 100, 200, 500, 1000 or 1500 consecutive nucleotides of a nucleic acid according to the invention.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ of 55°, can be used, e.g., 5×SSC, 0.1% SDS, 0.25% milk, and no formamide; or 30% formamide, 5×SSC, 0.5% SDS. Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra). For hybridization with shorter nucleic acids, i.e., oligonucleotides, the position of mismatches becomes more important, and the length of the oligonucleotide determines its specificity (see Sambrook et al., supra). The minimum length for a hybridizable nucleic acid may be at least about 10 nucleotides; at least about 15 nucleotides; or at least about 20 nucleotides.

In one embodiment, the term “standard hybridization conditions” refers to a Tₘ of 55° C., and utilizes conditions as set forth above. In another embodiment, the Tₘ is 60° C.; in another embodiment, the Tₘ is 65° C.

“High stringency hybridization conditions” for the purposes of the present invention will be understood to mean the following conditions:

1-Membrane competition and PREHYBRIDIZATION:

Mix: 40 μl salmon sperm DNA (10 mg/ml)

+40 μl human placental DNA (10 mg/ml)

Denature for 5 minutes at 96° C., then immerse the mixture in ice.

Remove the 2×SSC and pour 4 ml of formamide mix in the hybridization tube containing the membranes.

Add the mixture of the two denatured DNAs.

Incubation at 42° C. for 5 to 6 hours, with rotation.

2-Labeled probe competition:

Add to the labeled and purified probe 10 to 50 μl Cot I DNA, depending on the quantity of repeats.

Denature for 7 to 10 minutes at 95° C.

Incubate at 65° C. for 2 to 5 hours.

3-HYBRIDIZATION:

Remove the prehybridization mix.

Mix 40 μl salmon sperm DNA +40 μl human placental DNA; denature for 5 min at 96° C., then immerse in ice.

Add to the hybridization tube 4 ml of formamide mix, the mixture of the two DNAs and the denatured labeled probe/Cot I DNA.

Incubate 15 to 20 hours at 42° C., with rotation.

4-Washes and Exposure:

One wash at room temperature in 2×SSC, to rinse.

Wash twice 5 minutes at room temperature 2×SSC and 0.1% SDS at 65° C.

Wash twice 15 minutes 0.1×SSC and 0.1% SDS at 65° C.

Enclose the membranes in clear plastic wrap and expose.

The hybridization conditions described above are adapted to hybridization, under high stringency conditions, of a molecule of nucleic acid of varying length from 20 nucleotides to several hundreds of nucleotides. It goes without saying that the hybridization conditions described above may be adjusted as a function of the length of the nucleic acid whose hybridization is sought or of the type of labeling chosen, according to techniques known to one skilled in the art. Suitable hybridization conditions may, for example, be adjusted according to the teaching contained in the manual by Hames and Higgins (1985, supra).

As used herein, the term “oligonucleotide” refers to a nucleic acid, generally of at least 15 nucleotides, that is hybridizable to a nucleic acid according to the invention. Oligonucleotides can be labelled, e.g., with ³²P-nucleotides or nucleotides to which a label, such as biotin, has been covalently conjugated. In one embodiment, a labeled oligonucleotide can be used as a probe to detect the presence of a nucleic acid encoding an ABCA5-6, 9-10 polypeptide of the invention. In another embodiment, oligonucleotides (one or both of which may be labelled) can be used as PCR primers, either for cloning full lengths or fragments of any one of the ABCA5, ABCA6, ABCA9,and ABCA10 nucleic acids or to detect the presence of nucleic acids encoding any one of the ABCA5, ABCA6, ABCA9, and ABCA10 genes. In a further embodiment, an oligonucleotide of the invention can form a triple helix with any one of the ABCA5, ABCA6, ABCA9, and ABCA10 DNA molecules. Generally, oligonucleotides are prepared synthetically, for example, on a nucleic acid synthesizer. Accordingly, oligonucleotides can be prepared with non-naturally occurring phosphoester analog bonds, such as thioester bonds, etc.

“Homologous recombination” refers to the insertion of a foreign DNA sequence of a vector in a chromosome. Preferably, the vector targets a specific chromosomal site for homologous recombination. For specific-specific homologous recombination, the vector will contain sufficiently long regions of homology to sequences of the chromosome to allow complementary binding and incorporation of the vector into the chromosome. Longer regions of homology and greater degrees of sequence similarity may increase the efficiency of homologous recombination.

A DNA “coding sequence” is a double-stranded DNA sequence that is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

“Regulatory region” means a nucleic acid sequence which regulates the expression of a nucleic acid. A regulatory region may include sequences which are naturally responsible for expressing a particular nucleic acid (a homologous region) or may include sequences of a different origin (responsible for expressing different proteins or even synthetic proteins). In particular, the sequences can be sequences of eukaryotic or viral genes or derived sequences that stimulate or repress transcription of a gene in a specific or non-specific manner and in an inducible or non-inducible manner. Regulatory regions include origins of replication, RNA splice sites, enhancers, transcriptional termination sequences, signal sequences that direct the polypeptide into the secretory pathways of the target cell, and promoters.

A regulatory region from a “heterologous source” is a regulatory region that is not naturally associated with the expressed nucleic acid. Included among the heterologous regulatory regions are regulatory regions from a different species, regulatory regions from a different gene, hybrid regulatory sequences, and regulatory sequences that do not occur in nature, but which are designed by one having ordinary skill in the art.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined, for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

A “signal sequence” is included at the beginning of the coding sequence of a protein to be expressed on the surface of a cell. This sequence encodes a signal peptide, N-terminal to the mature polypeptide, that directs the host cell to translocate the polypeptide. The term “translocation signal sequence” is used herein to refer to this sort of signal sequence. Translocation signal sequences can be found associated with a variety of proteins native to eukaryotes and prokaryotes, and are often functional in both types of organisms.

A “polypeptide” is a polymeric compound comprised of covalently linked amino acid residues. Amino acids have the following general structure:

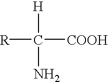

Amino acids are classified into seven groups on the basis of the side chain R: (1) aliphatic side chains, (2) side chains containing a hydroxyl (OH) group, (3) side chains containing sulfur atoms, (4) side chains containing an acidic or amide group, (5) side chains containing a basic group, (6) side chains containing an aromatic ring, and (7) proline, in which the side chain is fused to the amino group.

A “protein” is a polypeptide that plays a structural or functional role in a living cell.

The polypeptides and proteins of the invention may be glycosylated or unglycosylated.

“Homology” means similarity of sequence reflecting a common evolutionary origin. Polypeptides or proteins are said to have homology, or similarity, if a substantial number of their amino acids are either (1) identical, or (2) have a chemically similar R side chain. Nucleic acids are said to have homology if a substantial number of their nucleotides are identical.

“Isolated polypeptide” or “isolated protein” is a polypeptide or protein that is substantially free of those compounds that are normally associated therewith in its natural state (e.g., other proteins or polypeptides, nucleic acids, carbohydrates, lipids). “Isolated” is not meant to exclude artificial or synthetic mixtures with other compounds, or the presence of impurities which do not interfere with biological activity, and which may be present, for example, due to incomplete purification, addition of stabilizers, or compounding into a pharmaceutically acceptable preparation.

“Fragment” of a polypeptide according to the invention will be understood to mean a polypeptide whose amino acid sequence is shorter than that of the reference polypeptide and that comprises, over the entire portion with these reference polypeptides, an identical amino acid sequence. Such fragments may, where appropriate, be included in a larger polypeptide of which they are a part. Such fragments of a polypeptide according to the invention may have a length of about 5, about 10, about 15, about 20, about 30 to about 40, about 50, about 100, about 200 or about 300 amino acids.

“Variant” of a polypeptide according to the invention will be understood to mean mainly a polypeptide whose amino acid sequence contains one or more substitutions, additions, or deletions of at least one amino acid residue, relative to the amino acid sequence of the reference polypeptide, it being understood that the amino acid substitutions may be either conservative or nonconservative.

A “variant” of a polypeptide or protein is any analogue, fragment, derivative, or mutant that is derived from a polypeptide or protein and that retains at least one biological property of the polypeptide or protein. Different variants of the polypeptide or protein may exist in nature. These variants may result from allelic variations characterized by differences in the nucleotide sequences of the structural gene coding for the protein or may involve differential splicing or post-translational modification. Variants also include related proteins having substantially the same biological activity, but obtained from a different species.

The skilled artisan can produce variants having single or multiple amino acid substitutions, deletions, additions, or replacements. These variants may include, inter alia: (a) variants in which one or more amino acid residues are substituted with conservative or non-conservative amino acids, (b) variants in which one or more amino acids are added to the polypeptide or protein, (c) variants in which one or more of the amino acids includes a substituent group, and (d) variants in which the polypeptide or protein is fused with another polypeptide such as serum albumin. The techniques for obtaining these variants, including genetic (suppressions, deletions, mutations, etc.), chemical, and enzymatic techniques, are known to persons having ordinary skill in the art.

If such allelic variations, analogues, fragments, derivatives, mutants, and modifications, including alternative mRNA splicing forms and alternative post-translational modification forms, result in derivatives of the polypeptide that retain any of the biological properties of the polypeptide, they are intended to be included within the scope of this invention.

A “vector” is a replicon, such as plasmid, virus, phage, or cosmid to which another DNA segment may be attached so as to bring about the replication of the attached segment. A “replicon” is any genetic element (e.g., plasmid, chromosome, virus) that functions as an autonomous unit of DNA replication in vivo, i.e., is capable of replication under its own control.

The present invention also relates to cloning vectors containing genes encoding analogs and derivatives any of the ABCA5, ABCA6, ABCA9, and ABCA10 polypeptides of the invention that have the same or homologous functional activity as that of ABCA5, ABCA6, ABCA9, ABCA10 polypeptides and tp homologs thereof from other species. The production and use of derivatives and analogs related to the ABCA5, ABCA6, ABCA9, and ABCA10 polypeptides are within the scope of the present invention. In general, The derivatives or analogs are functionally active, i.e., capable of exhibiting one or more functional activities associated with the full-length, wild-type ABCA5, ABCA6, ABCA9, and ABCA10 polypeptides of the invention.

ABCA5, ABCA6, ABCA9, and ABCA10 derivatives can be made by altering encoding nucleic acid sequences by substitutions, additions or deletions that provide for functionally equivalent molecules. Preferably, derivatives are made that have enhanced or increased functional activity relative to native ABCA5, ABCA6, ABCA9, and ABCA10. Alternatively, such derivatives may encode soluble fragments of the ABCA5, ABCA6, ABCA9, and ABCA10 extracellular domains that have the same or greater affinity for the natural ligand of ABCA5, ABCA6, ABCA9, and ABCA10 polypeptides of the invention. Such soluble derivatives may be potent inhibitors of ligand binding to ABCA5, ABCA6, ABCA9, and ABCA10.

Due to the degeneracy of nucleotide coding sequences, other DNA sequences that encode substantially the same amino acid sequences as that of the ABCA5, ABCA6, ABCA9, and ABCA10 genes may be used in the practice of the present invention. These include, but are not limited to, allelic genes, homologous genes from other species, and nucleotide sequences comprising all or portions of ABCA5, ABCA6, ABCA9, and ABCA10 genes that are altered by the substitution of different codons that encode the same amino acid residue within the sequence, thereby producing a silent change. Likewise, the ABCA5, ABCA6, ABCA9, and ABCA10 derivatives of the invention include, but are not limited to, those containing, as a primary amino acid sequence, all or part of the amino acid sequence of any one of the ABCA5, ABCA6, ABCA9, and ABCA10 proteins, including altered sequences in which functionally equivalent amino acid residues are substituted for residues within the sequence resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan, and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Subtituition of one amino acid within a group for another is not expected to affect apparent molecular weight as determined by polyacrylamide gel electrophoresis, or isoelectric point.

Such substitutions include:

Lys for Arg and vice versa, such that a positive charge may be maintained;

Glu for Asp and vice versa, such that a negative charge may be maintained;

Ser for Thr, such that a free —OH can be maintained; and

Gln for Asn, such that a free CONH₂ can be maintained.

Amino acid substitutions may also be introduced to substitute an amino acid with a particularly desirable property. For example, a Cys may be introduced as a potential site for disulfide bridge formation with another Cys. A His may be introduced as a particularly “catalytic” site (i.e., His can act as an acid or base and is the most common amino acid in biochemical catalysis). Pro may be introduced because of its particularly planar structure, which induces b-turns in the protein's structure.

The genes encoding ABCA5, ABCA6, ABCA9, and ABCA10 derivatives and analogs of the invention can be produced by various methods known in the art. The manipulations that result in their production can occur at the gene or protein level. For example, the cloned ABCA5, ABCA6, ABCA9, and ABCA10 sequences can be modified by any of numerous strategies known in the art (Sambrook et al., 1989, supra). The sequence can be cleaved at appropriate sites with restriction endonuclease(s), followed by further enzymatic modification if desired, isolated, and ligated in vitro. Production of a gene encoding a derivative or analog of any one of the ABCA5, ABCA6, ABCA9, and ABCA10 should ensure that the modified gene remains within the same translational reading frame as the ABCA5, ABCA6, ABCA9, and ABCA10 genes, uninterrupted by translational stop signals in the region where the desired activity is encoded.

Additionally, the ABCA5, ABCA6, ABCA9, and ABCA10-encoding nucleic acids can be mutated in vitro or in vivo to create and/or destroy translation, initiation, and/or termination sequences or to create variations in coding regions and/or form new restriction endonuclease sites or destroy pre-existing ones to facilitate further in vitro modification. Such mutations may enhance the functional activity of the mutated ABCA5, ABCA6, ABCA9, and ABCA10 genes products. Any technique for mutagenesis known in the art may be used, including, inter alia, in vitro site-directed mutagenesis (Hutchinson et al., (1978) Biol. Chem. 253:6551; Zoller and Smith, (1984) DNA, 3:479-488; Oliphant et al., (1986) Gene 44:177; Hutchinson et al., (1986) Proc. Natl. Acad. Sci. U.S.A. 83:710; Huygen et al., (1996) Nature Medicine, 2(8):893-898) and use of TAB® linkers (Pharmacia). PCR techniques are preferred for site-directed mutagenesis (Higuchi, 1989, “Using PCR to Engineer DNA”, in PCR Technology: Principles and Applications for DNA Amplification, H. Erlich, ed., Stockton Press, Chapter 6, pp. 61-70).

Identified and isolated ABCA5, ABCA6, ABCA9, and ABCA10 genes may then be inserted into an appropriate cloning vector. A large number of vector-host systems known in the art may be used. Possible vectors include, but are not limited to, plasmids or modified viruses, but the vector system must be compatible with the host cell used. Examples of vectors for Escherichia coli include, but are not limited to, bacteriophages such as lambda derivatives, or plasmids such as pBR322 derivatives or pUC plasmid derivatives, e.g., PGEX vectors, pmal-c, pFLAG, etc. The insertion into a cloning vector can, for example, be accomplished by ligating the DNA fragment into a cloning vector that has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transformation, transfection, infection, electroporation, etc., so that many copies of the gene sequence are generated. The cloned gene may be contained on a shuttle vector plasmid, which provides for expansion in a cloning cell, e.g., Escherichia coli, and facile purification for subsequent insertion into an appropriate expression cell line, if such is desired. For example, a shuttle vector, which is a vector that can replicate in more than one type of organism, can be prepared for replication in both Escherichia coli and Saccharomyces cerevisiae by linking sequences from an Escherichia coil plasmid with sequences form the yeast 2m plasmid.

In an alternative method, the desired gene may be identified and isolated after insertion into a suitable cloning vector in a “shot gun” approach. Enrichment for the desired gene, for example, by size fractionation, can be done before insertion into the cloning vector.

The nucleotide sequences coding for the ABCA5, ABCA6, ABCA9, and ABCA10 polypeptides or antigenic fragments, derivatives, or analogs thereof, or functionally active derivatives, including chimeric proteins thereof, may be inserted into an appropriate expression vector, i.e., a vector that contains the necessary elements for the transcription and translation of the inserted protein-coding sequence. Such elements are termed herein a “promoter.” Thus, nucleic acids encoding the ABCA5, ABCA6, ABCA9, and ABCA10 polypeptides of the invention are operationally associated with a promoter in an expression vector of the invention. Both cDNA and genomic sequences can be cloned and expressed under control of such regulatory sequences. An expression vector also usually includes a replication origin.

The necessary transcriptional and translational signals can be provided on a recombinant expression vector, or they may be supplied by a native gene encoding ABCA5, ABCA6, ABCA9, and ABCA10 and/or its flanking regions.

Potential host-vector systems include, but are not limited to, mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A recombinant ABCA5, ABCA6, ABCA9, and ABCA10 protein of the invention, or functional fragments, derivatives, chimeric constructs, or analogs thereof, may be expressed chromosomally after integration of the coding sequence by recombination. In this regard, any of a number of amplification systems may be used to achieve high levels of stable gene expression (See Sambrook et al., 1989, supra).

The cell into which the recombinant vector comprising the nucleic acid encoding any one of the ABCA5, ABCA6, ABCA9, and ABCA10 polypeptides according to the invention is cultured in an appropriate cell culture medium under conditions that provide for expression of any one of the ABCA5, ABCA6, ABCA9, and ABCA10 polypeptides by the cell.

Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination (genetic recombination).

Expression of the ABCA5, ABCA6, ABCA9, and ABCA10 polypeptides may be controlled by any promoter/enhancer element known in the art, but these regulatory elements must be functional in the host selected for expression. Promoters that may be used to control ABCA5, ABCA6, ABCA9, and ABCA10 genes expression include, but are not limited to, the SV40 early promoter region (Benoist and Chambon, 1981 Nature 290:304-310), the promoter contained in the 3′ long terminal repeat (LTR) of Rous sarcoma virus (Yamamoto, et al., 1980 Cell 22:787-797), the herpes thymidine kinase promoter (Wagner et al., 1981 Proc. Natl. Acad. Sci. U.S.A. 78:1441-1445), the regulatory sequences of the metallothionein gene (Brinster et al., 1982 Nature 296:39-42); prokaryotic expression vectors such as the β-lactamase promoter (Villa-Kamaroff, et al., 1978 Proc. Natl. Acad. Sci. U.S.A. 75:3727-3731), or the tac promoter (DeBoer, et al., 1983 Proc. Natl. Acad. Sci. U.S.A. 80:21-25); see also “Useful proteins from recombinant bacteria” in Scientific American, 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region, which is active in pancreatic acinar cells (Swift et al., 1984 Cell 38:639-646; Ornitz et al., 1986 Cold Spring Harbor Symp. Quant. Biol. 50:399-409; MacDonald, 1987); insulin gene control region, which is active in pancreatic beta cells (Hanahan, 1985 Nature: 315:115-122), immunoglobulin gene control region, which is active in lymphoid cells (Grosschedl et al., 1984 Cell 38:647-658; Adames et al., 1985 Nature 318:533-538; Alexander et al., 1987 Mol. Cell. Biol. 7:1436-1444), mouse mammary tumor virus control region, which is active in testicular, breast, lymphoid, and mast cells (Leder et al., 1986 Cell 45:485-495), albumin gene control region, which is active in liver (Pinkert et al., 1987 Genes and Devel. 1:268-276), alpha-fetoprotein gene control region, which is active in liver (Krumlauf et al., 1985 Mol. Cell. Biol. 5:1639-1648; Hammer et al., 1987 Science 235:53-58), alpha 1-antitrypsin gene control region, which is active in the liver (Kelsey et al., 1987 Genes and Devel. 1:161-171) beta-globin gene control region, which is active in myeloid cells (Mogram et al., 1985 Nature 315:338-340; Kollias et al., 1986 Cell 46:89-94), myelin basic protein gene control region, which is active in oligodendrocyte cells in the brain (Readhead et al., 1987 Cell 48:703-712), myosin light chain-2 gene control region, which is active in skeletal muscle (Sani, 1985 Nature 314:283-286), and gonadotropic releasing hormone gene control region, which is active in the hypothalamus (Mason et al., 1986 Science 234:1372-1378).

Expression vectors containing a nucleic acid encoding one of the ABCA5, ABCA6, ABCA9, and ABCA10 polypeptides of the invention can be identified by five general approaches: (a) polymerase chain reaction (PCR) amplification of the desired plasmid DNA or specific mRNA, (b) nucleic acid hybridization, (c) presence or absence of selection marker gene functions, (d) analyses with appropriate restriction endonucleases, and (e) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In another example, if the nucleic acid encoding any one of the ABCA5, ABCA6, ABCA9, and ABCA10 polypeptides is inserted within the “selection marker” gene sequence of the vector, recombinants containing ABCA5, ABCA6, ABCA9, and ABCA10 nucleic acids inserts can be identified by the absence of the ABCA5, ABCA6, ABCA9, and ABCA10 gene functions. In the fourth approach, recombinant expression vectors are identified by digestion with appropriate restriction enzymes. In the fifth approach, recombinant expression vectors can be identified by assaying for the activity, biochemical, or immunological characteristics of the gene product expressed by the recombinant, provided that the expressed protein assumes a functionally active conformation.

A wide variety of host/expression vector combinations may be employed in expressing the nucleic acids of this invention. Useful expression vectors, for example, may consist of segments of chromosomal, non-chromosomal and synthetic DNA sequences. Suitable vectors include derivatives of SV40 and known bacterial plasmids, e.g., Escherichia coli plasmids col EI, pCR1, pBR322, pMaI-C2, pET, pGEX (Smith et al, 1988, Gene 67:31-40), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

For example, in a baculovirus expression system, both non-fusion transfer vectors, such as, but not limited to, pVL941 (BamH1 cloning site; Summers), pVL1393 (BamH1, SmaI, XbaI, EcoR1, NotI, XmaIII, BglII, and PstI cloning site; Invitrogen), pVL1392 (BgIII, PstI, NotI, XmaIII, EcoRI, XbaI, SmaI, and BamH1 cloning site; Summers and Invitrogen), and pBlueBacIII (BamH1, BglII, PstI, NcoI, and HindIII cloning site, with blue/white recombinant screening possible; Invitrogen), and fusion transfer vectors, such as, but not limited to, pAc700 (BamH1 and KpnI cloning site, in which the BamH1 recognition site begins with the initiation codon; Summers), pAc701 and pAc702 (same as pAc700, with different reading frames), pAc360 (BamH1 cloning site 36 base pairs downstream of a polyhedrin initiation codon; Invitrogen(195)), and pBlueBacHisA, B, C (three different reading frames, with BamH1, BglII, PstI, NcoI, and HindIII cloning site, an N-terminal peptide for ProBond purification, and blue/white recombinant screening of plaques; Invitrogen (220) can be used.

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR; See, Kaufman, Current Protocols in Molecular Biology, 16.12 (1991). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamH1, SfiI, XhoI, NotI, NheI, HindIII, NheI, PvuII, and KpnI cloning site, constitutive RSV-LTR promoter, hygromycin selectable marker; Invitrogen), pCEP4 (BamH1, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, XhoI, SfiI, BamH1 cloning site, inducible methallothionein IIa gene promoter, hygromycin selectable marker: Invitrogen), pREP8 (BamH1, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamH1 cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman, 1991, supra) for use according to the invention include, but are not limited to, pSC11 (Smal cloning site, TK- and b-gal selection), pMJ601 (SalI, SmaI, AflI, NarI, BspMII, BamHI, ApaI, NheI, SaclI, KpnI, and HindIII cloning site; TK- and b-gal selection), and pTKgptF1S (EcoRI, PstI, SalI, AccI, HindIII, SbaI, BamH1, and Hpa cloning site, TK or XPRT selection).

Yeast expression systems can also be used according to the invention to express any one of the ABCA5, ABCA6, ABCA9, and ABCA10 polypeptides. For example, the non-fusion pYES2 vector (XbaI, SphI, ShoI, NotI, GstXI, EcoRI, BstXI, BamH1, SacI, Kpn1, and HindIII cloning sit; Invitrogen) or the fusion pYESHisA, B, C (XbaI, SphI, ShoI, NotI, BstXI, EcoRI, BamH1, SacI, KpnI, and HindIII cloning site, N-terminal peptide purified with ProBond resin and cleaved with enterokinase; Invitrogen), to mention just two, can be employed according to the invention.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors that can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

In addition, a host cell strain may be chosen that modulates the expression of the inserted sequences or modifies and processes the gene product in the specific fashion desired. Different host cells have characteristic and specific mechanisms for the translational and post-translational processing and modification (e.g., glycosylation, cleavage for example of the signal sequence) of proteins. Appropriate cell lines or host systems can be chosen to ensure the desired modification and processing of the foreign protein expressed. For example, expression in a bacterial system can be used to produce a nonglycosylated core protein product. However, the transmembrane ABCA5, ABCA6, ABCA9, and ABCA10 proteins expressed in bacteria may not be properly folded. Expression in yeast can produce a glycosylated product. Expression in eukaryotic cells can increase the likelihood of “native” glycosylation and folding of a heterologous protein. Moreover, expression in mammalian cells can provide a tool for reconstituting, or constituting, ABCA5, ABCA6, ABCA9, and ABCA10 activities. Furthermore, different vector/host expression systems may affect processing reactions, such as proteolytic cleavages, to a different extent.

Vectors are introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

A cell has been “transfected” by exogenous or heterologous DNA when such DNA has been introduced inside the cell. A cell has been “transformed” by exogenous or heterologous DNA when the transfected DNA effects a phenotypic change. In one embodiment of the invention, the transforming DNA is integrated (covalently linked) into the chromosomal DNA making up the genome of the cell.

A recombinant marker protein expressed as an integral membrane protein can be isolated and purified by standard methods. Generally, the integral membrane protein can be obtained by lysing the membrane with detergents, such as but not limited to, sodium dodecyl sulfate (SDS), Triton X-100 polyoxyethylene ester, IpageI/nonidet P-40 (NP-40) (octylphenoxy)-polyethoxyethanol, digoxin, sodium deoxycholate, and the like, including mixtures thereof. Solubilization can be enhanced by sonication of the suspension. Soluble forms of the protein can be obtained by collecting culture fluid or by solubilizing inclusion bodies, e.g., by treatment with detergent, and, if desired, sonication or other mechanical processes, as described above. The solubilized or soluble protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

Alternatively, a nucleic acid or vector according to the invention can be introduced in vivo by lipofection. For the past decade, there has been increasing use of liposomes for encapsulation and transfection of nucleic acids in vitro. Synthetic cationic lipids designed to limit the difficulties and dangers encountered with liposome-mediated transfection can be used to prepare liposomes for in vivo transfection of a gene encoding a marker (Felgner, et. al. (1987. PNAS 84/7413); Mackey, et al. (1988. Proc. Natl. Acad. Sci. USA 85 :8027-8031); Ulmer et al. (1993. Science 259 :1745-1748). The use of cationic lipids may promote encapsulation of negatively charged nucleic acids and also promote fusion with negatively charged cell membranes (Felgner and Ringold, (1989. Science 337:387-388)). Particularly useful lipid compounds and compositions for transfer of nucleic acids are described in International Patent Publications W095/18863 and W096/17823, and in U.S. Pat. No. 5,459,127. The use of lipofection to introduce exogenous genes into the specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting [see Mackey, et. al., supra]. Targeted peptides, e.g., hormones or neurotransmitters, and proteins such as antibodies, or non-peptide molecules could be coupled to liposomes chemically.

Other molecules also are useful for facilitating transfection of a nucleic acid in vivo, such as a cationic oligopeptide (e.g., International Patent Publication WO95/21931), peptides derived from DNA binding proteins (e.g., International Patent Publication WO96/25508), or a cationic polymer (e.g., International Patent Publication WO95/21931).

It is also possible to introduce the vector in vivo as a naked DNA plasmid (see U.S. Pat. Nos. 5,693,622, 5,589,466, and 5,580,859). Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, Wu et al., 1992, supra; Wu and Wu, 1988, supra; Hartmut et al., Canadian Patent Application No. 2,012,311, filed Mar. 15, 1990; Williams et al., 1991, Proc. Natl. Acad. Sci. USA 88:2726-2730). Receptor-mediated DNA delivery approaches can also be used (Curiel et al., 1992, Hum. Gene Ther. 3:147-154; Wu and Wu, 1987, J. Biol. Chem. 262:4429-4432).

The term “pharmaceutically-acceptable vehicle or excipient” includes diluents and fillers which are pharmaceutically acceptable for method of administration, are sterile, and may be aqueous or oleaginous suspensions formulated using suitable dispersing or wetting agents and suspending agents. The particular pharmaceutically-acceptable carrier and the ratio of active compound to carrier are determined by the solubility and chemical properties of the composition, the particular mode of administration, and standard pharmaceutical practice.

Any nucleic acid, polypeptide, vector, or host cell of the invention will preferably be introduced in vivo in a pharmaceutically-acceptable vehicle or excipient. The phrase “pharmaceutically-acceptable” refers to molecular entities and compositions that are physiologically-tolerable and do not typically produce an allergic or similar untoward reaction, such as gastric upset, dizziness, and the like, when administered to a human. In general, the term “pharmaceutically-acceptable” means approved by a regulatory agency of the Federal or a state government or listed in the U.S. Pharmacopeia or other generally recognized pharmacopeia for use in animals, for example in humans. The term “excipient” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions may be employed as excipients, for example, for injectable solutions. Suitable pharmaceutical excipients are described in “Remington's Pharmaceutical Sciences” by E. W. Martin.

Naturally, the invention contemplates delivery of a vector that will express a therapeutically effective amount of any one of ABCA5, ABCA6, ABCA9, and ABCA10 polypeptides for gene therapy applications. The phrase “therapeutically effective amount” is used herein to mean an amount sufficient to reduce by at least about 15 percent, at least 50 percent, at least 90 percent, or even prevent a clinically significant deficit in the activity, function, and response of the host. Alternatively, a therapeutically effective amount is sufficient to cause an improvement in a clinically significant condition in the host.

“Lipid profile” means the set of concentrations of cholesterol, triglyceride, lipoprotein cholesterol and other lipids in the body of a human or other animal.

An “undesirable lipid profile” is the condition in which the concentrations of cholesterol, triglyceride, or lipoprotein cholesterol are outside of the age- and gender-adjusted reference ranges. Generally, a concentration of total cholesterol>200 mg/dl, of plasma triglycerides>200 mg/dl, of LDL cholesterol>130 mg/dl, of HDL cholesterol<39 mg/dl, or a ratio of total cholesterol to HDL cholesterol>4.0 is considered to be an undesirable lipid profile. An undesirable lipid profile is associated with a variety of pathological conditions, including hyperlipidaemias, diabetes hypercholesterolaemia, arteriosclerosis, and other forms of coronary artery disease.

Nucleic Acids of the ABCA5, A6, A9, A10 Genes

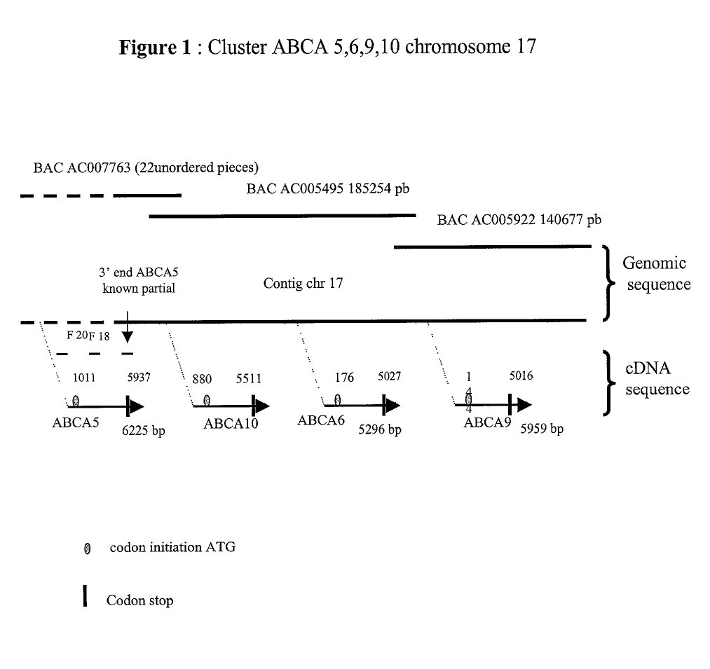

Applicants have identified a novel human ABCA-like cluster of genes, designated ABCA5, ABCA6, ABCA9, and ABCA10. Applicants also have determined that the new genes are closely spaced and arranged head-to-tail in the following order ABCA5, ABCA10, ABCA6, and ABCA9 on same region of chromosome 17q24 and encode full transporters (FIG. 1).

Applicants also have determined that each new ABCA gene has a unique expression pattern, suggesting that the corresponding proteins may perform tissue-specialized functions (Example 3).

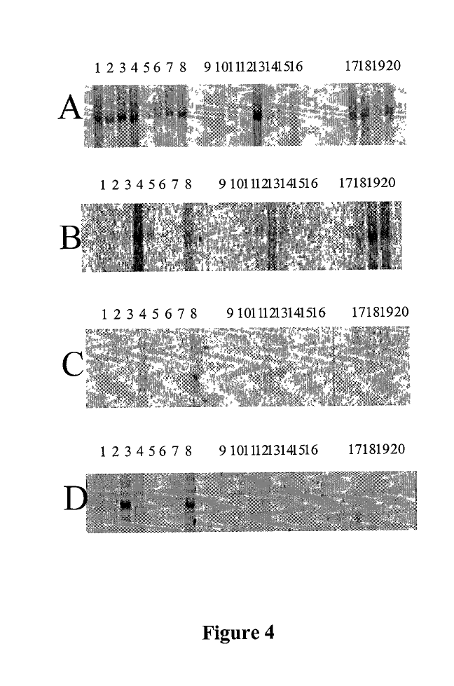

The expression patterns showed that the 6.5 kb ABCA5 transcript is almost ubiquitous, but the strongest expression was found in testis, skeletal muscle, fetal kidney and fetal liver. The ABCA9 transcript of 6 kb was found in heart and a weak signal was detected in the ovary, small intestine and testis. The ABCA6 transcript of 7 kb-long was detected with a strong signal in the liver and fetal liver, a weaker signal was detected in heart, kidney (fetal and adult), lung (fetal and adult), colon, small intestine, ovary, and testis. No signal was detected under the same conditions in the brain (fetal and adult), pancreas, placenta, skeletal muscle, leukocyte, pancreas, spleen, or thymus. The ABCA10 transcript of 6.5 kb was strongly and specifically detected in skeletal muscle and heart (FIG. 4).

Also, in situ hybridization showed the strongest ABCA9 gene expression in endothelial cells, vascular smooth muscle, and Schwann cells, and the strongest ABCA10 gene expression was identified consistently in macrophages, subsets of lymphocytes, and in Schwann cells of nerves.

Applicants have further determined transcript sequences that correspond to the full coding sequence (CDS) of the ABCA5, ABCA6, and ABCA9, and ABCA10 genes and that the ABCA6 and ABCA9 genes comprise 39 exons and 38 introns, and the ABCA10 gene comprises at least 40 exons and 39 introns. Table 1 hereinafter presents splice donors and acceptors scores (Rᵢ, bits) that are consistent with that of exons in other mammalian genes (Rogan et al., Hum Mutat (1998) 12, 153-171). Exons are located in exactly the same positions in all genes, although the length of some of the exons varies. Furthermore, there is a high correlation coefficient (0.990-0.997) for exon size between these genes and significant correlations (0.27-0.64) for some of the comparisons of intron sizes and Rᵢ values, clearly suggesting that the genes from the 17q24 cluster arose by duplication from a common ancestor.

TABLE 1
Correlations of exon size, intron size and Ri values.

| | Exons | Introns | Ri-SD | Ri-SA |
|---|---|---|---|---|
| ABCA6-ABCA9 | 0.997 | 0.62 | 0.45 | 0.28 |
| ABCA6-ABCA10 | 0.990 | 0.46 | 0.64 | 0.53 |
| ABCA9-ABCA10 | 0.995 | 0.43 | 0.47 | 0.46 |

Applicants have thus characterized new exon sequences of the human ABCA6, ABCA9, and ABCA10 genes, which are particularly useful according to the invention for detecting the corresponding, ABCA6, ABCA9, and ABCA10 genes or nucleotide expression products in a sample.

Several exons of ABCA6 gene have been characterized by their nucleotide sequence and are identified in Table 2.

TABLE 2
Human ABCA6 exons and intron DNA

| Exon or | Exon start in | Exon stop in | Exon | Exon | Length | Intron start | Intron stop | Length |
|---|---|---|---|---|---|---|---|---|
| intron | genomic | genomic | start in | stop in | of | in genomic | in genomic | in |
| number | fragment | fragment | mRNA | mRNA | exon | fragment | fragment | intron |
| 1 | 123426 | 123555 | 1 | 130 | 130 | 123556 | 124551 | 996 |
| 2 | 124552 | 124692 | 131 | 271 | 141 | 124693 | 127799 | 3107 |
| 3 | 127800 | 128004 | 272 | 476 | 205 | 128005 | 129049 | 1045 |
| 4 | 129050 | 129208 | 477 | 635 | 159 | 129209 | 130557 | 1349 |
| 5 | 130558 | 130661 | 636 | 739 | 104 | 130662 | 131432 | 771 |
| 6 | 131433 | 131659 | 740 | 966 | 227 | 131660 | 135548 | 3889 |
| 7 | 135549 | 135690 | 967 | 1108 | 142 | 135691 | 136495 | 805 |
| 8 | 136496 | 136681 | 1109 | 1294 | 186 | 136682 | 140264 | 3583 |
| 9 | 140265 | 140412 | 1295 | 1442 | 148 | 140413 | 141892 | 1480 |
| 10 | 141893 | 142061 | 1443 | 1611 | 169 | 142062 | 147343 | 5282 |
| 11 | 147344 | 147402 | 1612 | 1670 | 59 | 147403 | 149813 | 2411 |
| 12 | 149814 | 149924 | 1671 | 1781 | 111 | 149925 | 150362 | 438 |
| 13 | 150363 | 150538 | 1782 | 1957 | 176 | 150539 | 151562 | 1024 |
| 14 | 151563 | 151682 | 1958 | 2077 | 120 | 151683 | 151939 | 257 |
| 15 | 151940 | 152078 | 2078 | 2216 | 139 | 152079 | 153026 | 948 |
| 16 | 153027 | 153117 | 2217 | 2307 | 90 | 153118 | 154359 | 1242 |
| 17 | 154360 | 154499 | 2308 | 2447 | 139 | 154500 | 157487 | 2988 |
| 18 | 157488 | 157604 | 2448 | 2564 | 117 | 157605 | 159088 | 1484 |
| 19 | 159089 | 159272 | 2565 | 2748 | 184 | 159273 | 159671 | 399 |
| 20 | 159672 | 159838 | 2749 | 2915 | 167 | 159839 | 162331 | 2493 |
| 21 | 162332 | 162465 | 2916 | 3049 | 134 | 162466 | 164365 | 1900 |
| 22 | 154366 | 164503 | 3050 | 3187 | 138 | 164504 | 167272 | 2769 |
| 23 | 167273 | 167380 | 3188 | 3295 | 108 | 167381 | 168498 | 1118 |
| 24 | 168499 | 168672 | 3296 | 3469 | 174 | 168673 | 168946 | 274 |
| 25 | 168947 | 169060 | 3470 | 3583 | 114 | 169061 | 174037 | 4977 |
| 26 | 174038 | 174157 | 3584 | 3703 | 120 | 174158 | 175757 | 1600 |
| 27 | 175758 | 175835 | 3704 | 3781 | 78 | 175836 | 177041 | 1206 |
| 28 | 177042 | 177133 | 3782 | 3873 | 92 | 177134 | 177826 | 693 |
| 29 | 177827 | 177947 | 3874 | 3994 | 121 | 177948 | 178564 | 617 |
| 30 | 178565 | 178682 | 3995 | 4112 | 118 | 178683 | 179583 | 901 |
| 31 | 179584 | 179675 | 4113 | 4204 | 92 | 179676 | 180117 | 442 |
| 32 | 180118 | 180272 | 4205 | 4359 | 155 | 180273 | 180792 | 520 |
| 33 | 180793 | 180868 | 4360 | 4435 | 76 | 180869 | 180944 | 76 |
| 34 | 180945 | 181039 | 4436 | 4530 | 95 | 181040 | 181968 | 929 |
| 35 | 181969 | 182088 | 4531 | 4650 | 120 | 182089 | 182286 | 198 |
| 36 | 182287 | 182427 | 4651 | 4791 | 141 | 182428 | 184154 | 1727 |
| 37 | 184155 | 184234 | 4792 | 4871 | 80 | 184235 | 186034 | 1800 |
| 38 | 186035 | 186090 | 4872 | 4927 | 56 | 186091 | 186225 | 135 |
| 39 | 186226 | 186594 | 4928 | 5296 | 369 | 186595 | | |

Thus the present invention also relates to a nucleic acid comprising any one of SEQ ID NOs: 9-47 or a complementary sequence.

The invention also relates to a nucleic acid comprising a nucleotide sequence as depicted in any one of SEQ ID NOs: 9-47 or a complementary nucleotide sequence.

The invention also relates to a nucleic acid comprising at least 8 consecutive nucleotides of any one of SEQ ID NOs: 9-47 or a complementary nucleotide sequence.

The subject of the invention is, in addition, a nucleic acid having at least 80% nucleotide identity with a nucleic acid comprising any one of SEQ ID NOs: 9-47 or a complementary nucleotide sequence.

The invention also relates to a nucleic acid having at least 85%, at least 90%, at least 95%, or at least 98% nucleotide identity with a nucleic acid comprising any one of SEQ ID NOs: 9-47.

The invention also relates to a nucleic acid hybridizing, under high stringency conditions, with a nucleic acid comprising any one of SEQ ID NOs: 9-47 or a complementary nucleotide sequence.

Several exons of the ABCA9 gene have been characterized by their nucleotide sequence and are identified in Table 3.

TABLE 3
Human ABCA9 exons and intron DNA

| Exon or | Exon start in | Exon stop in | Exon | Exon | Length | Intron start | Intron stop | Length |
|---|---|---|---|---|---|---|---|---|
| intron | genomic | genomic | start in | stop in | of | in genomic | in genomic | in |
| number | fragment | fragment | mRNA | mRNA | exon | fragment | fragment | intron |
| 1 | 204305 | 204434 | 1 | 130 | 130 | 204435 | 214160 | 9726 |
| 2 | 214161 | 214269 | 131 | 239 | 109 | 214270 | 215809 | 1540 |
| 3 | 215810 | 216017 | 240 | 447 | 208 | 216018 | 219963 | 3946 |
| 4 | 219964 | 220128 | 448 | 612 | 165 | 220129 | 220699 | 571 |
| 5 | 220700 | 220803 | 613 | 716 | 104 | 220804 | 221584 | 781 |
| 6 | 221585 | 221811 | 717 | 943 | 227 | 221812 | 229498 | 7687 |
| 7 | 229499 | 229640 | 944 | 1085 | 142 | 229641 | 229868 | 228 |
| 8 | 229869 | 230054 | 1086 | 1271 | 186 | 230055 | 231426 | 1372 |
| 9 | 231427 | 231574 | 1272 | 1419 | 148 | 231575 | 233023 | 1449 |
| 10 | 233024 | 233192 | 1420 | 1588 | 169 | 233193 | 236072 | 2880 |
| 11 | 236073 | 236131 | 1589 | 1647 | 59 | 236132 | 236654 | 523 |
| 12 | 236655 | 236765 | 1648 | 1758 | 111 | 236766 | 237484 | 719 |
| 13 | 237485 | 237660 | 1759 | 1934 | 176 | 237661 | 237850 | 190 |
| 14 | 237851 | 237970 | 1935 | 2054 | 120 | 237971 | 238185 | 215 |
| 15 | 238186 | 238324 | 2055 | 2193 | 139 | 238325 | 238832 | 508 |
| 16 | 238833 | 238923 | 2194 | 2284 | 91 | 238924 | 240946 | 2023 |
| 17 | 240947 | 241086 | 2285 | 2424 | 140 | 241087 | 243438 | 2352 |
| 18 | 243439 | 243558 | 2425 | 2544 | 120 | 243559 | 244713 | 1155 |
| 19 | 244714 | 244912 | 2545 | 2743 | 199 | 244913 | 246720 | 1808 |
| 20 | 246721 | 246887 | 2744 | 2910 | 167 | 246888 | 247510 | 623 |
| 21 | 247511 | 247644 | 2911 | 3044 | 134 | 247645 | 248909 | 1265 |
| 22 | 248910 | 249047 | 3045 | 3182 | 138 | 249048 | 253216 | 4169 |
| 23 | 253217 | 253324 | 3183 | 3290 | 108 | 253325 | 257064 | 3740 |
| 24 | 257065 | 257238 | 3291 | 3464 | 174 | 257239 | 257427 | 189 |
| 25 | 257428 | 257641 | 3465 | 3578 | 114 | 257542 | 269285 | 11744 |
| 26 | 269286 | 269405 | 3579 | 3698 | 120 | 269406 | 272215 | 2810 |
| 27 | 272216 | 272284 | 3699 | 3767 | 69 | 272285 | 273033 | 749 |
| 28 | 273034 | 273125 | 3768 | 3859 | 92 | 273126 | 274342 | 1217 |
| 29 | 274343 | 274463 | 3860 | 3980 | 121 | 274464 | 275369 | 906 |
| 30 | 275370 | 275487 | 3981 | 4098 | 118 | 275488 | 276181 | 694 |
| 31 | 276182 | 276273 | 4099 | 4190 | 92 | 276274 | 278975 | 2702 |
| 32 | 278976 | 279136 | 4191 | 4351 | 161 | 279137 | 280171 | 1035 |
| 33 | 280172 | 280247 | 4352 | 4427 | 76 | 280248 | 280320 | 73 |
| 34 | 280321 | 280415 | 4428 | 4522 | 95 | 280416 | 281124 | 709 |
| 35 | 281125 | 281244 | 4523 | 4642 | 120 | 281245 | 281450 | 206 |
| 36 | 281451 | 281591 | 4643 | 4783 | 141 | 281592 | 282658 | 1067 |
| 37 | 282659 | 282738 | 4784 | 4863 | 80 | 282739 | 289109 | 6371 |
| 38 | 289110 | 289165 | 4864 | 4919 | 56 | 289166 | 289286 | 121 |
| 39 | 289287 | 290352 | 4920 | 5981 | 1062 | 290353 | | |

Thus, the present invention also relates to a nucleic acid comprising any one of SEQ ID NOs: 48-86 or a complementary sequence.

The invention also relates to a nucleic acid comprising a nucleotide sequence as depicted in any one of SEQ ID NOs: 48-86, or a complementary nucleotide sequence.

The invention also relates to a nucleic acid comprising at least 8 consecutive nucleotides of any one of SEQ ID NOs: 48-86 or a complementary nucleotide sequence.

The subject of the invention is, in addition, a nucleic acid having at least 80% nucleotide identity with a nucleic acid comprising any one of SEQ ID NOs: 48-86 or a complementary nucleotide sequence.

The invention also relates to a nucleic acid having at least 85%, at least 90%, at least 95%, or at least 98% nucleotide identity with a nucleic acid comprising any one of SEQ ID NOs: 48-86.

The invention also relates to a nucleic acid hybridizing, under high stringency conditions, with a nucleic acid comprising any one of SEQ ID NOs: 48-86 or a complementary nucleotide sequence.

Several exons of the ABCA10 gene have been characterized by their nucleotide sequence and are identified in Table 4.

TABLE 4
Human ABCA10 exons and introns DNA

| Exon or | Exon start in | Exon stop in | Exon | Exon | Length | Intron start | Intron stop | Length |
|---|---|---|---|---|---|---|---|---|
| intron | genomic | genomic | start in | stop in | of | in genomic | in genomic | in |
| number | fragment | fragment | mRNA | mRNA | exon | fragment | fragment | intron |
| 1 | 20483 | 20769 | 1 | 287 | 287 | 20770 | 36437 | 15668 |
| 2 | 36438 | 36717 | 288 | 567 | 280 | 36718 | 38012 | 1295 |
| 3 | 38013 | 38153 | 568 | 708 | 141 | 38154 | 39768 | 1615 |
| 4 | 39769 | 39973 | 709 | 913 | 205 | 39974 | 42600 | 2627 |
| 5 | 42601 | 42765 | 914 | 1078 | 165 | 42766 | 43402 | 637 |
| 6 | 43403 | 43506 | 1079 | 1182 | 104 | 43507 | 45526 | 2020 |
| 7 | 45527 | 45753 | 1183 | 1409 | 227 | 45754 | 48939 | 3186 |
| 8 | 48940 | 49081 | 1410 | 1551 | 142 | 49082 | 49297 | 216 |
| 9 | 49298 | 49483 | 1552 | 1737 | 186 | 49484 | 50446 | 963 |
| 10 | 50447 | 50594 | 1738 | 1885 | 148 | 50595 | 63629 | 13035 |
| 11 | 63630 | 63798 | 1886 | 2054 | 169 | 63799 | 68175 | 4377 |
| 12 | 68176 | 68234 | 2055 | 2113 | 59 | 68235 | 70803 | 2569 |
| 13 | 70804 | 70914 | 2114 | 2224 | 111 | 70915 | 71309 | 395 |
| 14 | 71310 | 71485 | 2225 | 2400 | 176 | 71486 | 71686 | 201 |
| 15 | 71687 | 71806 | 2401 | 2520 | 120 | 71807 | 72050 | 244 |
| 16 | 72051 | 72189 | 2521 | 2659 | 139 | 72190 | 72645 | 456 |
| 17 | 72646 | 72736 | 2660 | 2750 | 91 | 72737 | 73983 | 1247 |
| 18 | 73984 | 74123 | 2751 | 2890 | 140 | 74124 | 74821 | 698 |
| 19 | 74822 | 74941 | 2891 | 3010 | 120 | 74942 | 77419 | 2478 |
| 20 | 77420 | 77618 | 3011 | 3209 | 199 | 77619 | 79655 | 2037 |
| 21 | 79656 | 79822 | 3210 | 3376 | 167 | 79823 | 82490 | 2668 |
| 22 | 82491 | 82624 | 3377 | 3510 | 134 | 82625 | 83008 | 384 |
| 23 | 83009 | 83146 | 3511 | 3648 | 138 | 83147 | 89785 | 6639 |
| 24 | 89786 | 89893 | 3649 | 3756 | 108 | 89894 | 90521 | 628 |
| 25 | 90522 | 90692 | 3757 | 3927 | 171 | 90693 | 90904 | 212 |
| 26 | 90905 | 91018 | 3928 | 4041 | 114 | 91019 | 100216 | 9198 |
| 27 | 100217 | 100336 | 4042 | 4161 | 120 | 100337 | 101145 | 809 |
| 28 | 101146 | 101226 | 4162 | 4242 | 81 | 101227 | 108376 | 7150 |
| 29 | 108377 | 108468 | 4243 | 4334 | 92 | 108469 | 109374 | 906 |
| 30 | 109375 | 109495 | 4335 | 4455 | 121 | 109496 | 110163 | 668 |
| 31 | 110164 | 110281 | 4456 | 4573 | 118 | 110282 | 110973 | 692 |
| 32 | 110974 | 111065 | 4574 | 4665 | 92 | 111066 | 111290 | 225 |
| 33 | 111291 | 111469 | 4666 | 4844 | 179 | 111470 | 111753 | 284 |
| 34 | 111754 | 111829 | 4845 | 4920 | 76 | 111830 | 111900 | 71 |
| 35 | 111901 | 111995 | 4921 | 5015 | 95 | 111996 | 112818 | 823 |
| 36 | 112819 | 112938 | 5016 | 5135 | 120 | 112939 | 113116 | 178 |
| 37 | 113117 | 113257 | 5136 | 5276 | 141 | 113258 | 115236 | 1979 |
| 38 | 115237 | 115316 | 5277 | 5356 | 80 | 115317 | 116211 | 895 |
| 39 | 116212 | 116267 | 5357 | 5412 | 56 | 116268 | 116374 | 107 |
| | 116375 | 117143 | 5413 | 6181 | 769 | 117144 | | |

Thus, the invention also relates to a nucleic acid comprising any one of SEQ ID NOs: 87-126 or a complementary nucleotide sequence thereof.

The invention also relates to a nucleic acid comprising a nucleotide sequence as depicted in any one of SEQ ID NOs: 87-126 or a complementary nucleotide sequence thereof.

The invention also relates to a nucleic acid comprising at least 8 consecutive nucleotides of any one of SEQ ID NOs: 87-126 or a complementary nucleotide sequence.

The subject of the invention is, in addition, a nucleic acid having at least 80% nucleotide identity with a nucleic acid comprising any one of SEQ ID NOs: 87-126 or a complementary nucleotide sequence.

The invention also relates to a nucleic acid having at least 85%, at least 90%, at least 95%, or at least 98% nucleotide identity with a nucleic acid comprising any one of SEQ ID NOs: 87-126 or a complementary nucleotide sequence.

The invention also relates to a nucleic acid hybridizing, under high stringency conditions, with a nucleic acid comprising any one of SEQ ID NOs: 87-126 or a complementary nucleotide sequence.

cDNA Molecules Encoding Full Length ABCA5, ABCA6, ABCA9, and ABCA10 Proteins

Applicants have further determined the cDNA sequences and the full coding sequences (CDS) of the human ABCA5, A6, A9, and A10 genes, which belong to the same chromosome 17 cluster and encode full length human corresponding proteins (Example 2).

Table 5 summarizes, for each gene, the mRNA length, the coding nucleotide sequence length, and the protein size.

TABLE 5
Characterization of the four ABCA on the chromosome 17 cluster

| | mRNA | | | | Number |
|---|---|---|---|---|---|
| | length | CDS | Polyadenylation | Protein | of coding |
| | (bp) | (bp) | site | (AA) | exons |
| ABCA5 | 6525 | 4929 | — | 1642 | Nd* |
| ABCA6 | 5296 | 4854 | AATAAA | 1617 | 38 |
| | | | (position 5284) | | |
| ABCA9 | 5959 | 4875 | — | 1624 | 38 |
| ABCA10 | 6181 | 4632 | — | 1543 | 37 |

Nd*: not determined; the genomic sequence is in progress.

The cDNA sequence of ABCA5 comprises 6525 nucleotides and contains a 4929 nucleotide coding sequence corresponding to a 1642 amino acid (aa) ABCA5 polypeptide produced in subjects not affected by disorders associated with cholesterol reverse transport or inflammatory lipid mediators transport. The cDNA molecule of the novel human ABCA5 gene having the nucleotide sequence as set forth in SEQ ID NO: 1 comprises an open reading frame beginning from the nucleotide at position 1011 (base A of the ATG codon for initiation of translation) to the nucleotide at position 5939 (base A of the TGA stop codon).

According to the invention, the ABCA5 cDNA comprising SEQ ID NO: 1 encodes a full length ABCA5 polypeptide of 1642 amino acids comprising the amino acid sequence of SEQ ID NO: 5.

The cDNA molecule of the novel human ABCA6 gene having the nucleotide sequence as set forth in SEQ ID NO: 2 comprises an open reading frame beginning from the nucleotide at position 176 (base A of the ATG codon for initiation of translation) to the nucleotide at position 5029 (second base A of the TAA stop codon). A polyadenylation signal (having the sequence AATAAA) is present, starting from the nucleotide at position 5284 of the sequence SEQ ID NO: 2.

According to the invention, the ABCA6 cDNA (SEQ ID NO: 2) comprises 5296 nucleotides and contains a 4854 nucleotide coding sequence that encodes a full length ABCA6 polypeptide of 1617 amino acids comprising the amino acid sequence of SEQ ID NO: 6.

The cDNA molecule of the novel human ABCA9 gene having the nucleotide sequence as set forth in SEQ ID NO: 3 comprises a coding sequence beginning from the nucleotide at position 144 (base A of the ATG codon for initiation of translation) to the nucleotide at position 5018 (second base A of the TAA stop codon).

According to the invention, the ABCA9 cDNA (SEQ ID NO: 3) comprises 5959 nucleotides and contains a 4875 nucleotide coding sequence which encodes a full length ABCA9 polypeptide of 1624 amino acids comprising the amino acid sequence of SEQ ID NO: 7.

The cDNA molecule of the novel human ABCA10 gene having the nucleotide sequence as set forth in SEQ ID NO: 4 comprises a coding sequence beginning from the nucleotide at position 880 (base A of the ATG codon for initiation of translation) to the nucleotide at position 5511 (second base A of the TAA stop codon).

According to the invention, the ABCA10 cDNA (SEQ ID NO: 4) comprises 6181 nucleotides and contains a 4632 nucleotide coding sequence which encodes a full length ABCA10 polypeptide of 1543 amino acids comprising the amino acid sequence of SEQ ID NO: 8.

The present invention is directed to a nucleic acid comprising SEQ ID NOs: 1-4 or a complementary nucleotide sequence thereof.

The invention also relates to a nucleic acid comprising a nucleotide sequence as depicted in SEQ ID NO :1-4 or a complementary nucleotide sequence thereof.

The invention also relates to a nucleic acid comprising at least eight consecutive nucleotides of SEQ ID NOS: 1-4 or a complementary nucleotide sequence thereof.

The subject of the invention is also a nucleic acid having at least 80% nucleotide identity with a nucleic acid comprising nucleotides of SEQ ID NOs: 1-4 or a nucleic acid having a complementary nucleotide sequence thereof.

The invention also relates to a nucleic acid having at least 85%, at least 90%, at least 95%, or at least 98% nucleotide identity with a nucleic acid comprising a nucleotides of SEQ ID NOs: 1-4 or a nucleic acid having a complementary nucleotide sequence thereof.

Another subject of the invention is a nucleic acid hybridizing, under high stringency conditions, with a nucleic acid comprising nucleotides of SEQ ID NOs: 1-4 or a nucleic acid having a complementary nucleotide sequence thereof.

The invention also relates to a nucleic acid encoding a polypeptide comprising an amino acid sequence of SEQ ID NOs: 5-8.

The invention relates to a nucleic acid encoding a polypeptide comprising an amino acid sequence as depicted in SEQ ID NOs: 5-8.

The invention also relates to a polypeptide comprising an amino acid sequence of SEQ ID NOs: 5-8.

The invention also relates to a polypeptide comprising anamino acid sequence as depicted in SEQ ID NOs: 5-8.

The invention also relates to a polypeptide comprising an amino acid sequence having at least 80% amino acid identity with a polypeptide comprising an amino acid sequence of SEQ ID NOs: 5-8 or a peptide fragment thereof.

The invention also relates to a polypeptide having at least 85%, at least 90%, at least 95%, or at least 98% amino acid identity with a polypeptide comprising an amino acid sequence of SEQ ID NOs: 5-8.

Preferably, a polypeptide according to the invention will have a length of 4, 5 to 10, 15, 18 or 20 to 25, 35, 40, 50, 70, 80, 100 or 200 consecutive amino acids of a polypeptide according to the invention comprising an amino acid sequence of SEQ ID NOs: 5-8.

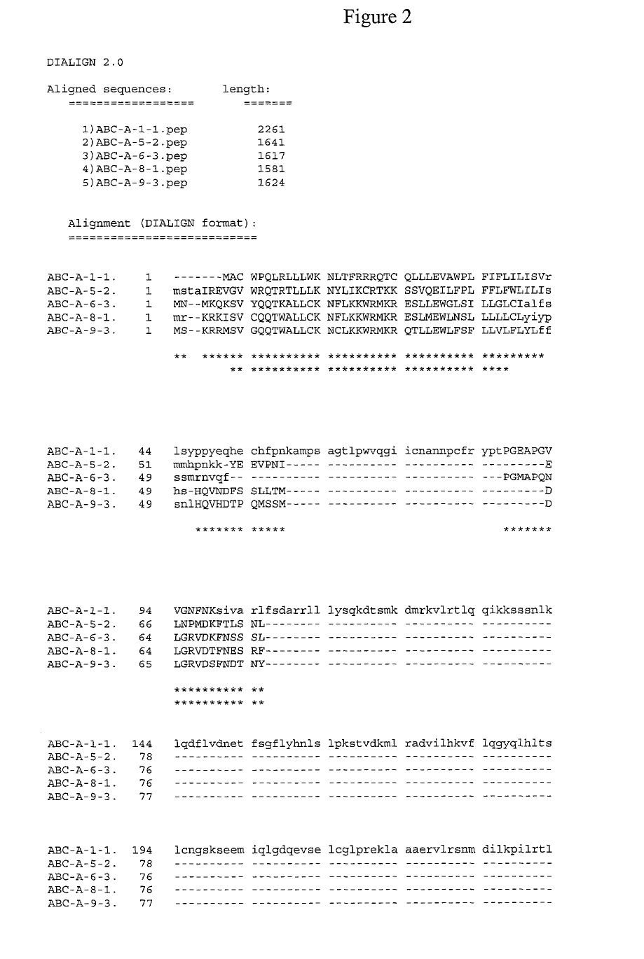

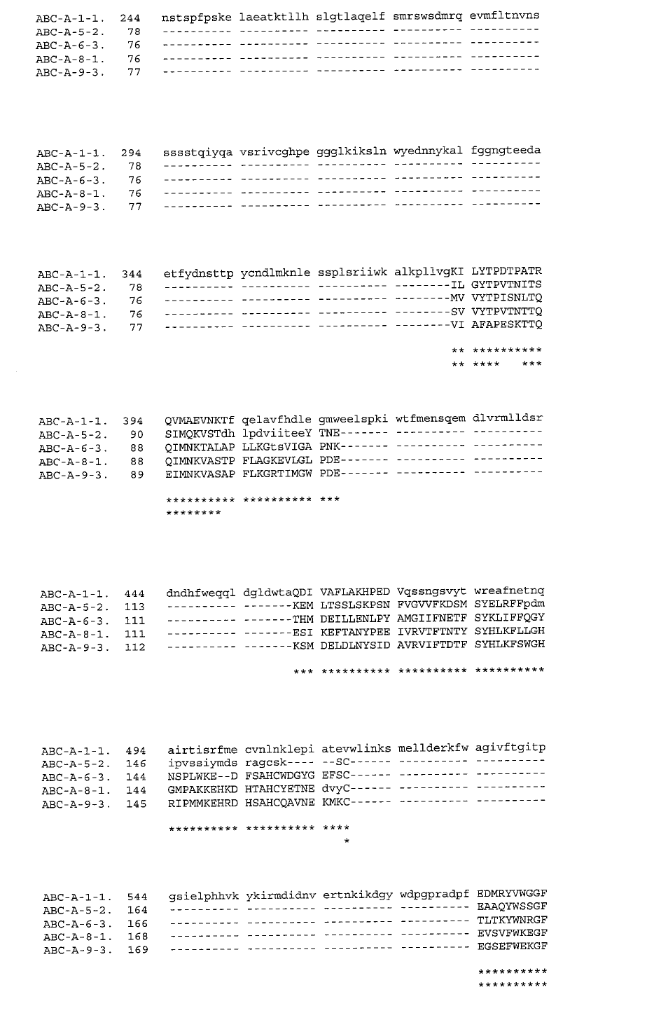

Like the ABCA1 and ABCA4 transporters, which present 45 to 66% amino acid sequences identity, the ABCA5, ABCA6, ABCA9, and ABCA10 proteins also demonstrate high conservation as set forth in Tables 6-10 (FIG. 2). Alignment of the amino acid sequences of the ABCA5, ABCA6, ABCA9 and ABCA10 genes reveals an identity ranging from 43 to 62% along the entire sequence (Table 6). Particularly, the ABCA5, ABCA6, ABCA9, and ABCA10 proteins show 32 to 60% and 34 to 48% identity in the N-terminal (Table 7) and C-terminal (Table 8) trans-membrane domains (TMC and TMN), respectively, and 56 to 77% identity in the ATP-binding domains (NBD1 and NBD2; Tables 9 and 10).

TABLE 6
Homology/Identity percentages between the amino acid sequences
of ABCA5, ABCA6, ABCA8, ABCA9, ABCA10, and ABCA1 along the
entire sequence

| Total sequence | ABCA5 | ABCA6 | ABCA8 | ABCA9 | ABCA10 | ABCA1 |
|---|---|---|---|---|---|---|
| ABCA5 | 100/100 | | | | | |
| ABCA6 | 52.9/42.8 | 100/100 | | | | |
| ABCA8 | 52.4/42.4 | 67/59.7 | 100/100 | | | |
| ABCA9 | 52.6/42.7 | 67.4/59.4 | 78.2/71.6 | 100/100 | | |
| ABCA10 | 53.2/43.4 | 69.5/62.3 | 68.1/61.1 | 70.3/62.1 | 100/100 | |
| ABCA1 | 41.5/30.8 | 42.8/31 | 42.832 | 41.1/30.9 | 41.2/30.6 | 100/100 |

TABLE 7
Homology/Identity percentages between the amino acid sequences
of ABCA5, ABCA6, ABCA9, ABCA10, ABCA1, and ABCA8 in the N
terminal transmembrane domain

| TMN domain | ABCA5 | ABCA6 | ABCA8 | ABCA9 | ABCA10 | ABCA1 |
|---|---|---|---|---|---|---|
| ABCA5 | 100/100 | | | | | |
| ABCA6 | 47/34.2 | 100/100 | | | | |
| ABCA8 | 46.5/35 | 70.2/59.1 | 100/100 | | | |
| ABCA9 | 46.3/37.8 | 64.2/55.7 | 76.9/68.5 | 100/100 | | |
| ABCA10 | 43.4/32.3 | 68.5/60.4 | 70.7/60.8 | 65.5/57.9 | 100/100 | |
| ABCA1 | 36.5/23.1 | 34.2/20 | 39.8/27.6 | 40.6/27.9 | 35.4/24.4 | 100/100 |

TABLE 8
Homology/Identity percentages between the amino acid sequences
of ABCA5, ABCA6, ABCA9, ABCA10, ABCA1 and ABCA8 in the C terminal
transmembrane domain

| TMC domain | ABCA5 | ABCA6 | ABCA8 | ABCA9 | ABCA10 | ABCA1 |
|---|---|---|---|---|---|---|
| ABCA5 | 100/100 | | | | | |
| ABCA6 | 43.7/33.7 | 100/100 | | | | |
| ABCA8 | 48.2/31.8 | 53.8/44.2 | 100/100 | | | |
| ABCA9 | 47.3/33.7 | 57.2/48.2 | 64.1/52.9 | 100/100 | | |
| ABCA10 | 47/35.4 | 57/47 | 54.3/43 | 57.4/44.4 | 100/100 | |
| ABCA1 | 33/21.6 | 32/21.4 | 39/24.8 | 35.3/26.8 | 34.7/22.4 | 100/100 |

TABLE 9
Homology/Identity percentages between the amino acid sequences
of ABCA5, ABCA6, ABCA9, ABCA10, ABCA1, and ABCA8 in the nucleotide
Binding Domain 1 (NBD1)

| NBD1 domain | ABCA5 | ABCA6 | ABCA8 | ABCA9 | ABCA10 | ABCA1 |
|---|---|---|---|---|---|---|
| ABCA5 | 100/100 | | | | | |
| ABCA6 | 70.2/60.3 | 100/100 | | | | |
| ABCA8 | 71.8/62.5 | 85.4/78.6 | 100/100 | | | |
| ABCA9 | 65.5/58.2 | 80.2/72.8 | 88.5/81.8 | 100/100 | | |
| ABCA10 | 69.8/62.1 | 83.2/77.2 | 82.8/79.2 | 81.9/75.8 | 100/100 | |
| ABCA1 | 56.8/48.5 | 53.5/43.5 | 61.2/50.5 | 51.7/43.8 | 56.5/45.6 | 100/100 |

TABLE 10
Homology/Identity percentages between the amino acid sequences
of ABCA5, ABCA6, ABCA9, ABCA10, ABCA1, and ABCA8 in the nucleotide
Binding Domain 2 (NBD2)

| NBD2 domain | ABCA5 | ABCA6 | ABCA8 | ABCA9 | ABCA10 | ABCA1 |
|---|---|---|---|---|---|---|
| ABCA5 | 100/100 | | | | | |
| ABCA6 | 63/56.1 | 100/100 | | | | |
| ABCA8 | 66.9/58.4 | 78/73.5 | 100/100 | | | |
| ABCA9 | 65.2/57.0 | 77.6/72.6 | 94.5/91.8 | 100/100 | | |
| ABCA10 | 63.7/56.2 | 74.9/71.2 | 81/77.4 | 82.3/77.8 | 100/100 | |
| ABCA1 | 46.4/37.8 | 46.3/37.9 | 46.9/38 | 47.3/39 | 46.4/37.7 | 100/100 |

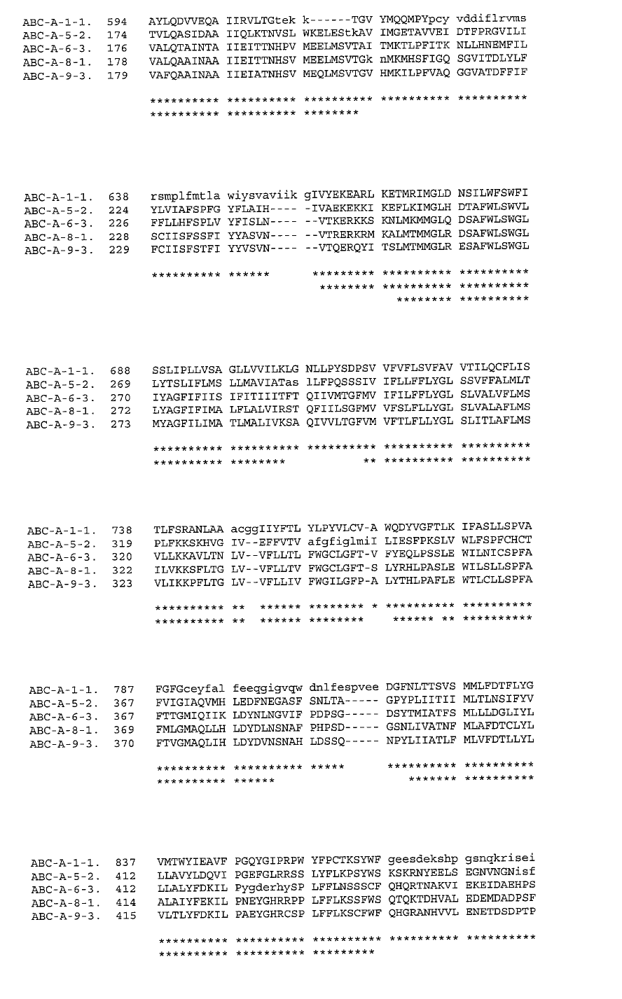

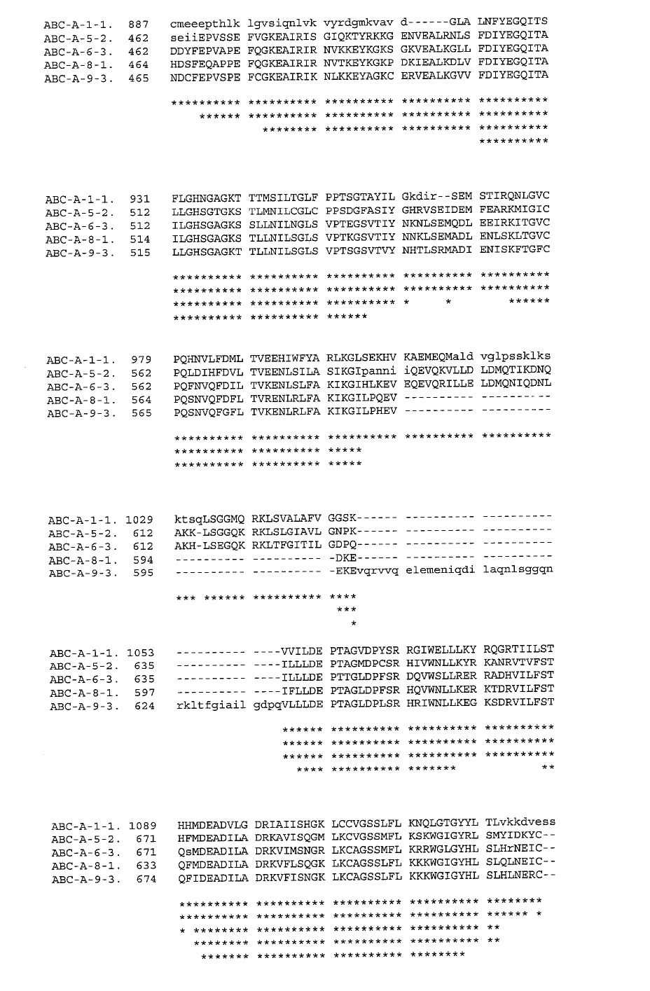

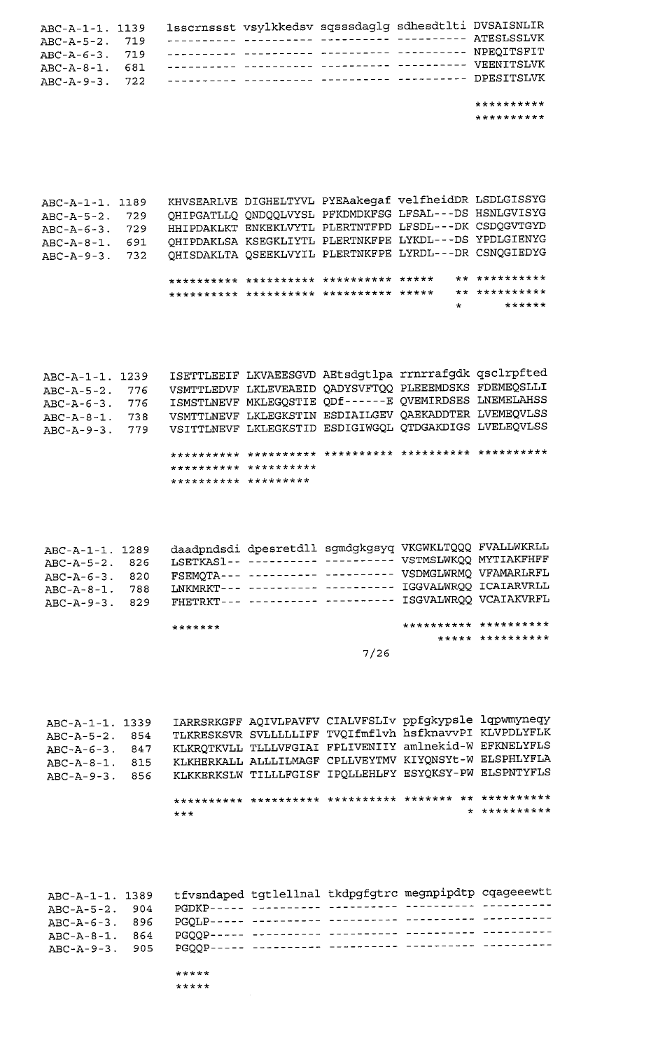

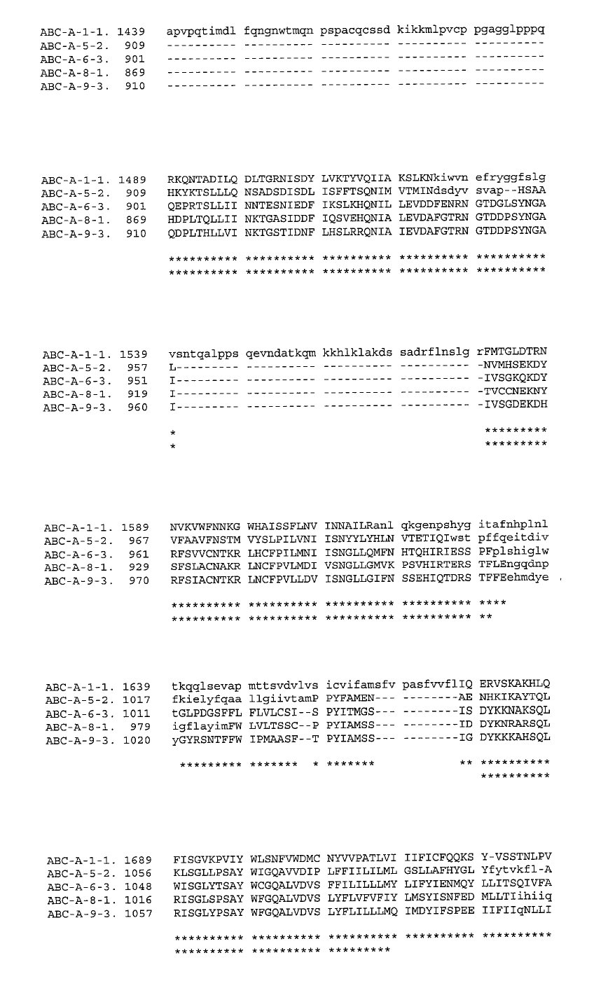

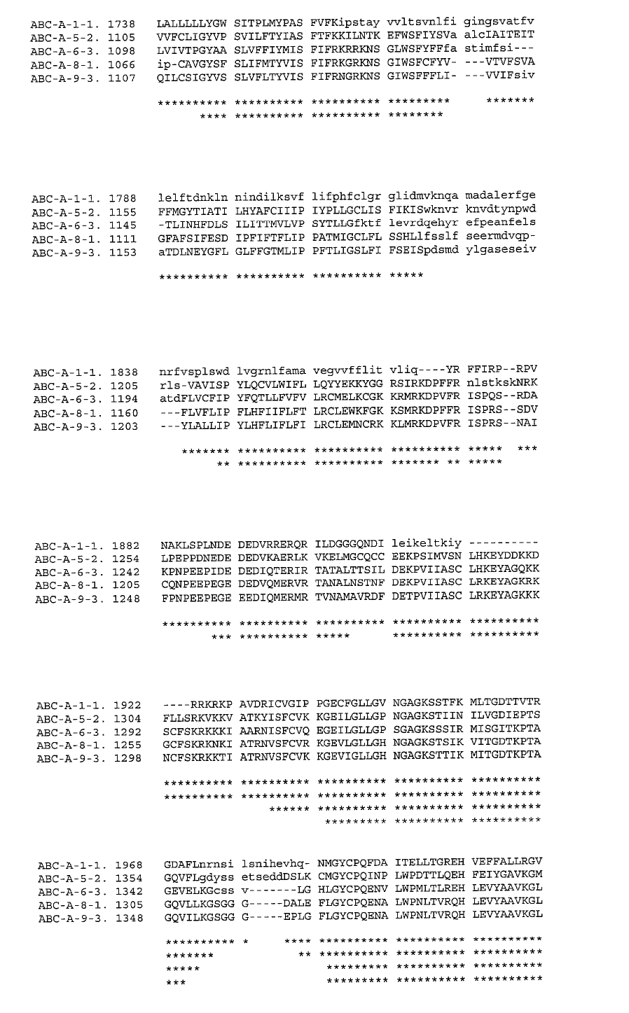

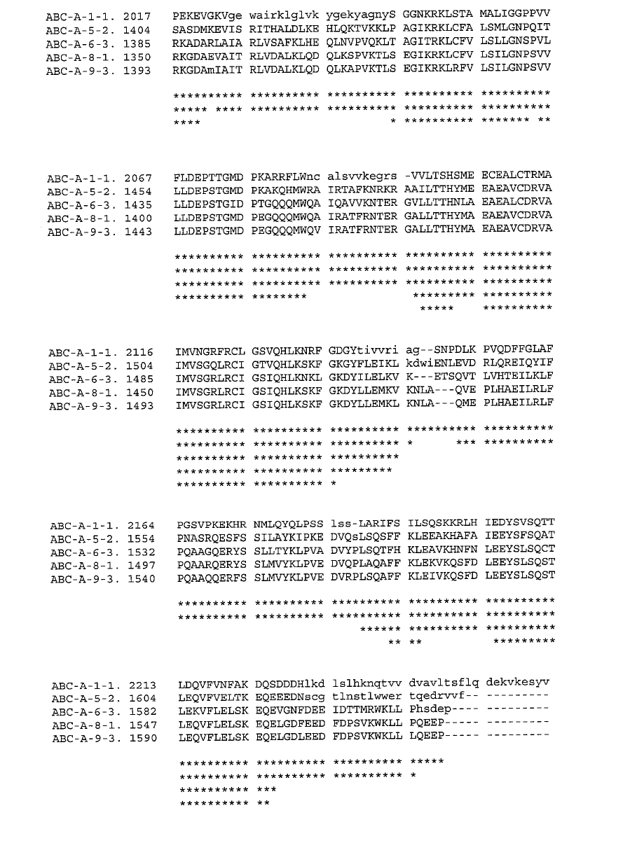

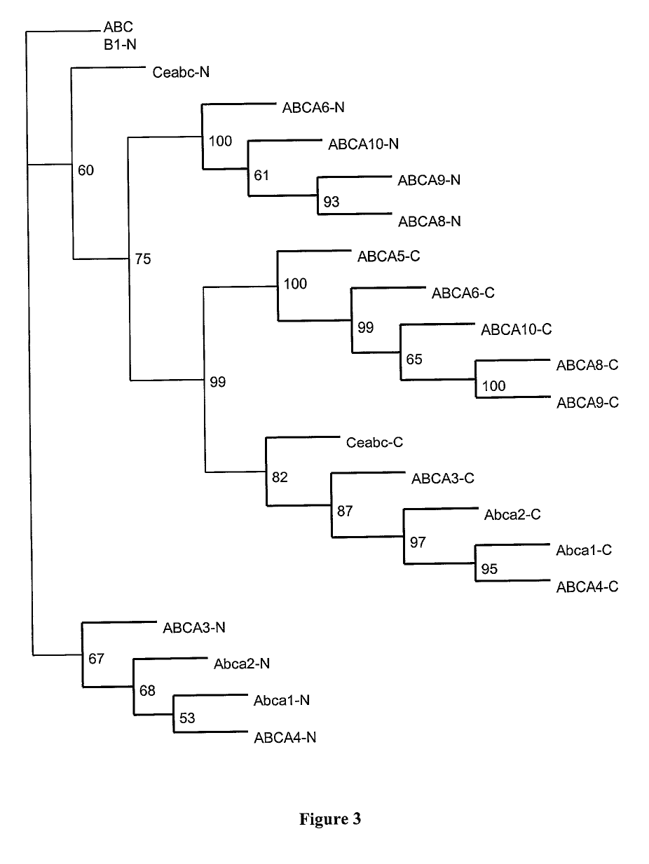

Phylogenetic analysis of the ATP-binding domains demonstrated that the N- and C-terminal domains form separate branches (FIG. 3). The C-terminal ATP-binding domains of the 17q24 genes are more closely related to the C-terminal domains of the other ABC1-like genes than to the N-terminal domains of the same proteins. Thus, the entire ABC1 subfamily appears to have arisen from a single ancestral full transporter gene. However, the genes in the 17q24 cluster form a distinct group within the ABC1 subfamily.

Nucleotide Probes and Primers

Nucleotide probes and primers hybridizing with a nucleic acid (genomic DNA, messenger RNA, cDNA) according to the invention also form part of the invention.

According to the invention, nucleic acid fragments derived from a polynucleotide comprising any one of SEQ ID NOs: 1-4 and 9-126 or of a complementary nucleotide sequence are useful for the detection of the presence of at least one copy of a nucleotide sequence of any one of the ABCA5, ABCA6, ABCA9, and ABCA10 genes or of a fragment or of a variant (containing a mutation or a polymorphism) thereof in a sample.

The nucleotide probes or primers according to the invention comprise a nucleotide sequence comprising any one of SEQ ID NOs: 1-4 and 9-126 or a complementary nucleotide sequence.

The nucleotide probes or primers according to the invention comprise at least 8 consecutive nucleotides of a nucleic acid comprising any one of SEQ ID NOs: 1-4 and 9-126 or a complementary nucleotide sequence.

Nucleotide probes or primers according to the invention may have a length of about 10, about 12, about 15, about 18 or about 20 to about 25, about 35, about 40, about 50, about 70, about 80, about 100, about 200, about 500, about 1000, or about 1500 consecutive nucleotides of a nucleic acid according to the invention, for example, a nucleic acid comprising any one of SEQ ID NOs: 1-4 and 9-126 or a complementary nucleotide sequence.

Alternatively, a nucleotide probe or primer according to the invention consists of and/or comprise the fragments having a length of about 12, about 15, about 18, about 20, about 25, about 35, about 40, about 50, about 100, about 200, about 500, about 1000, or about 1500 consecutive nucleotides of a nucleic acid according to the invention, for example, a nucleic acid comprising any one of SEQ ID NOs: 1-4 and 9-126 or a complementary nucleotide sequence.

The definition of a nucleotide probe or primer according to the invention therefore encompasses oligonucleotides that hybridize, under the high stringency hybridization conditions defined above, with a nucleic acid comprising any one of SEQ ID NOs: 1-4 and 9-126, or a complementary nucleotide sequence.

According to a preferred embodiment, a nucleotide primer according to the invention comprises a nucleotide sequence of any one of SEQ ID NOs: 127-144, or a complementary nucleic acid sequence.

Examples of primers and pairs of primers that make it possible to amplify various regions of the ABCA5 gene are presented in Table 11 below. The location of each primer of SEQ ID NOs: 127-144 within SEQ ID NO: 1 and its hybridizing region is indicated in Table 11. The abbreviation “Comp” refers to the complementary nucleic acid sequence.

TABLE 11
Primers for the amplification of nucleic fragments
of the ABCA5 gene

| Primer | Located in | Position in |
|---|---|---|
| SEQ ID NO: | SEQ ID NO: | the sequence |
| 127 | 1 | 3842-3860 |
| 128 | 1 | Comp 4858-4876 |
| 129 | 1 | Comp 4783-4801 |
| 130 | 1 | Comp 5789-5807 |
| 131 | 1 | 5630-5648 |
| 132 | 1 | 4858-4876 |
| 133 | 1 | Comp 3998-4016 |
| 134 | 1 | Comp 2987-3005 |
| 135 | 1 | Comp 3186-3208 |
| 136 | 1 | 2528-2547 |
| 137 | 1 | Comp 3088-3107 |
| 138 | 1 | Comp 2528-2547 |
| 139 | 1 | Comp 845-862 |
| 140 | 1 | 789-807 |
| 141 | 1 | Comp 1614-1633 |
| 142 | 1 | 1614-1633 |
| 143 | 1 | Comp 537-566 |
| 144 | 1 | Comp 202-231 |

According to one embodiment, a nucleotide primer according to the invention comprises a nucleotide sequence of any one of SEQ ID NOs: 145-172 or a complementary nucleic acid sequence.

Examples of primers and pairs of primers that make it possible to amplify various regions of the ABCA6 gene are presented in Table 12 below. The location of each primer of SEQ ID NOs: 145-172 within SEQ ID NO: 2 and its hybridizing region is indicated in Table 12. The abbreviation “Comp” refers to the complementary nucleic acid sequence.

TABLE 12
Primers for the amplification of nucleic fragments of
the ABCA6 gene

| Primer | Located in | Position in | Region |
|---|---|---|---|
| SEQ ID NO: | SEQ ID NO: | the sequence | for hybridization |
| 145 | 2 | 202-221 | Exon 2 |
| 146 | 2 | Comp 435-461 | Exon 3 |
| 147 | 2 | Comp 645-672 | Exon 5 |
| 148 | 2 | 637-656 | Exon 5 |
| 149 | 2 | 754-772 | Exon 6 |
| 150 | 2 | Comp 758-778 | Exon 6 |
| 151 | 2 | Comp 773-792 | Exon 6 |
| 152 | 2 | 1288-1307 | Exon 8-9 |
| 153 | 2 | 1321-1341 | Exon 9 |
| 154 | 2 | Comp 1322-1343 | Exon 9 |
| 155 | 2 | Comp 1592-1574 | Exon 10 |
| 156 | 2 | 1761-1782 | Exon 12 |
| 157 | 2 | Comp 1928-1949 | Exon 13 |
| 158 | 2 | 1944-1968 | Exon 13-14 |
| 159 | 2 | Comp 2041-2061 | Exon 14 |
| 160 | 2 | Comp 2371-2392 | Exon 17 |
| 161 | 2 | 2350-2371 | Exon 17 |
| 162 | 2 | 2806-2884 | Exon 20 |
| 163 | 2 | Comp 2884-2902 | Exon 20 |
| 164 | 2 | 3292-3313 | Exon 23-24 |
| 165 | 2 | Comp 3357-3339 | Exon 24 |
| 166 | 2 | Comp 3746-3767 | Exon 27 |
| 167 | 2 | 3754-3775 | Exon 27 |
| 168 | 2 | 4176-4194 | Exon 31 |
| 169 | 2 | Comp 4248-4194 | Exon 31-32 |
| 170 | 2 | 4743-4763 | Exon 36 |
| 171 | 2 | Comp 4796-4778 | Exon 36-37 |
| 172 | 2 | Comp 5262-5244 | Exon 39 |

According another embodiment, a nucleotide primer according to the invention comprises a nucleotide sequence of any one of SEQ ID NOs: 173-203 or a complementary nucleic acid sequence.

Examples of primers and pairs of primers that make it possible to amplify various regions of the ABCA9 gene are presented in Table 13 below. The location of each primer of SEQ ID NOs: 173-203 within SEQ ID NO: 3 and its hybridizing region is indicated in Table 13. The abbreviation “Comp” refers to the complementary nucleic acid sequence.

TABLE 13
Primers for the amplification of nucleic fragments
of the ABCA9 gene

| Primer | Located in | Position in | Region |
|---|---|---|---|
| SEQ ID NO: | SEQ ID NO: | the sequence | for hybridization |
| 173 | 3 | 160-178 | Exon 2 |
| 174 | 3 | Comp 789-808 | Exon 6 |
| 175 | 3 | 786-804 | Exon 6 |
| 176 | 3 | Comp 1434-1455 | Exon 10 |
| 177 | 3 | 1305-1323 | Exon 9 |
| 178 | 3 | Comp 1632-1653 | Exon 11-12 |
| 179 | 3 | 1495-1516 | Exon 10 |
| 180 | 3 | 1866-1887 | Exon 13 |
| 181 | 3 | Comp 1905-1923 | Exon 13 |
| 182 | 3 | Comp 2349-2368 | Exon 17 |
| 183 | 3 | 2253-2272 | Exon 17 |
| 184 | 3 | Comp 2822-2843 | Exon 20 |
| 185 | 3 | 2645-2663 | Exon 19 |
| 186 | 3 | Comp 3089-3110 | Exon 22 |
| 187 | 3 | 3240-3260 | Exon 23 |
| 188 | 3 | 3023-3044 | Exon 21 |
| 189 | 3 | Comp 3801-3820 | Exon 28 |
| 190 | 3 | Comp 3377-3398 | Exon 24 |
| 191 | 3 | 3626-3646 | Exon 26 |
| 192 | 3 | Comp 4191-4209 | Exon 32 |
| 193 | 3 | 3964-3984 | Exon 29-30 |
| 194 | 3 | Comp 4784-4803 | Exon 37 |
| 195 | 3 | 5230-5247 | Exon 39 |
| 196 | 3 | 4694-4715 | Exon 36 |
| 197 | 3 | Comp 4977-4994 | Exon 39 |
| 198 | 3 | 5541-5561 | Exon 39 |
| 199 | 3 | Comp 5960-5981 | Exon 39 |
| 200 | 3 | Comp 5541-5562 | Exon 39 |
| 201 | 3 | 24-45 | Exon 1 |
| 202 | 3 | Comp 384-408 | Exon 3 |
| 203 | 3 | Comp 311-337 | Exon 3 |

According to another embodiment, a nucleotide primer according to invention comprises a nucleotide sequence of any one of SEQ ID NOs: 204-217 complementary nucleic acid sequence thereof.

Examples of primers and pairs of primers that make it possible to amplify various regions of the ABCA10 gene are presented in Table 14 below. The location of each primer of SEQ ID NOs: 204-217 within SEQ ID NO: 4 and its hybridizing region is indicated in Table 14. The abbreviation “Comp” refers to the complementary nucleic acid sequence.

TABLE 14
Primers for the amplification of nucleic fragments of the
ABCA10 gene

| Primer | Located in | Position in | Region |
|---|---|---|---|
| SEQ ID NO: | SEQ ID NO: | the sequence | for hybridization |
| 204 | 4 | 1421-1440 | Exon 8 |
| 205 | 4 | Comp 1610-1629 | Exon 9 |
| 206 | 4 | 2417-2434 | Exon 15 |
| 207 | 4 | Comp 2605-2623 | Exon 16 |
| 208 | 4 | Comp 3737-3754 | Exon 24 |
| 209 | 4 | Comp 814-839 | Exon 4 |
| 210 | 4 | Comp 733-757 | Exon 4 |
| 211 | 4 | 61-86 | Exon 1 |
| 212 | 4 | 628-643 | Exon 3 |
| 213 | 4 | 3564-3583 | Exon 23 |
| 214 | 4 | Comp 4450-4468 | Exon 30-31 |
| 215 | 4 | Comp 5442-5459 | Exon 40 |
| 216 | 4 | 3050-3070 | Exon 20 |
| 217 | 4 | Comp 4848-4866 | Exon 34 |

According to another embodiment, probes and primers according to invention comprise all or part of a nucleotide sequence comprising any one of SEQ ID NOs: 127-217 or a nucleic acid having a complementary nucleic acid sequence.

A nucleotide primer or probe according to the invention may be prepared by any suitable method well known to persons skilled in the art, including by cloning and action of restriction enzymes or by direct chemical synthesis according to techniques such as the phosphodiester method by Narang et al. (1979, Methods Enzymol, 68:90-98) or by Brown et al. (1979, Methods Enzymol, 68:109-151), the diethylphosphoramidite method by Beaucage et al. (1981, Tetrahedron Lett, 22: 1859-1862), or the technique on a solid support described in EU patent No. EP 0,707,592.

Each of the nucleic acids according to the invention, including the oligonucleotide probes and primers described above, may be labeled, if desired, by incorporating a marker which can be detected by spectroscopic, photochemical, biochemical, immunochemical or chemical means. For example, such markers may consist of radioactive isotopes (³²P, ³³P, ³H, ³⁵S), fluorescent molecules (5-bromodeoxyuridine, fluorescein, acetylaminofluorene, digoxigenin) or ligands such as biotin. The labeling of the probes may be carried out by incorporating labeled molecules into the polynucleotides by primer extension or, alternatively, by addition to the 5′ or 3′ ends. Examples of nonradioactive labeling of nucleic acid fragments are described in particular in French patent No. 78 109 75 or in the articles by Urdea et al. (1988, Nucleic Acids Research, 11:4937-4957) or Sanchez-pescador et al. (1988, J. Clin. Microbiol., 26(10):1934-1938).

The nucleotide probes and primers according to the invention may have structural characteristics of the type to allow amplification of the signal, such as the probes described by Urdea et al. (1991, Nucleic Acids Symp Ser., 24:197-200) or alternatively in European patent No. EP-0,225,807 (CHIRON).

The oligonucleotide probes according to the invention may be used, for example, in Southern-type hybridizations with genomic DNA or, alternatively, in northern-type hybridizations with the corresponding messenger RNA when the expression of the corresponding transcript is sought in a sample.

The probes and primers according to the invention may also be used for the detection of products of PCR amplification or, alternatively, for the detection of mismatches.

Nucleotide probes or primers according to the invention may be immobilized on a solid support. Such solid supports are well known to persons skilled in the art and comprise surfaces of wells of microtiter plates, polystyrene beads, magnetic beads, nitrocellulose bands, or microparticles such as latex particles.

Consequently, the present invention also relates to a method of detecting the presence of a nucleic acid comprising a nucleotide sequence of any one of SEQ ID NOs: 1-4 and 9-126 or of a complementary nucleotide sequence, or a nucleic acid fragment or variant of any one of SEQ ID NOs: 1-4 and 9-126 or of a complementary nucleotide sequence in a sample, said method comprising:

1) bringing one or more nucleotide probes or primers according to the invention into contact with the sample to be tested;

2) detecting the complex that may have formed between the probe(s) and the nucleic acid present in the sample.

According to one embodiment of the method of detection according to the invention, the oligonucleotide probes and primers are immobilized on a support.

According to another aspect, the oligonucleotide probes and primers comprise a detectable marker.

The invention relates, in addition, to a box or kit for detecting the presence of a nucleic acid according to the invention in a sample, said box or kit comprising:

a) one or more nucleotide probe(s) or primer(s) as described above;

b) where appropriate, the reagents necessary for the hybridization reaction.

According to one aspect, the detection box or kit is characterized in that the probe(s) or primer(s) are immobilized on a support.

According to another aspect, the detection box or kit is characterized in that the oligonucleotide probes comprise a detectable marker.

According to another embodiment of the detection kit described above, such a kit comprises a plurality of oligonucleotide probes and/or primers in accordance with the invention that may be used to detect a target nucleic acid of interest or, alternatively, to detect mutations in the coding regions and/or in the non-coding regions of the nucleic acids according to the invention, for example, of nucleic acids comprising any one of SEQ ID NOs: 1-4 and 9-126 or a complementary nucleotide sequence.

Thus, the probes according to the invention immobilized on a support may be ordered into matrices such as “DNA chips”. Such ordered matrices have been described in U.S. Pat. No. 5,143,854 and in published PCT applications WO 90/15070 and WO 92/10092.

Support matrices on which oligonucleotide probes have been immobilized at a high density are, for example, described in U.S. Pat. No. 5,412,087 and in published PCT application WO 95/11995.

The nucleotide primers according to the invention may be used to amplify any one of the nucleic acids according to the invention, for example, a nucleic acid comprising a nucleotide sequence of any one of SEQ ID NOs: 1-4 and 9-126 or of a complementary nucleotide sequence. Alternatively, the nucleotide primers according to the invention may be used to amplify a nucleic acid fragment or variant of any one of SEQ ID NOs: 1-4 and 9-126 or of a complementary nucleotide sequence.

In one embodiment, the nucleotide primers according to the invention may be used to amplify a nucleic acid comprising any one of SEQ ID NOs: 1-4 and 9-126, or as depicted in any one of SEQ ID NOs: 1-4 and 9-126, or of a complementary nucleotide sequence.

Another subject of the invention relates to a method for amplifying a nucleic acid according to the invention, for example, a nucleic acid comprising a) any one of SEQ ID NOs: 1-4 and 9-126 or a complementary nucleotide sequence or b) as depicted in any one of SEQ ID NOs:1-4 and 9-126 or of a complementary nucleotide sequence, contained in a sample, said method comprising:

a) bringing the sample in which the presence of the target nucleic acid is suspected into contact with a pair of nucleotide primers whose hybridization position is located, respectively, on the 5′ side and on the 3′ side of the region of the target nucleic acid whose amplification is sought, in the presence of the reagents necessary for the amplification reaction;

b) performing an amplification reaction; and

c) detecting the amplified nucleic acids.

To carry out the amplification method as defined above, use may be made of any of the nucleotide primers described above.

The subject of the invention is, in addition, a box or kit for amplifying a nucleic acid according to the invention, for example, a nucleic acid comprising any one of SEQ ID NOs: 1-4 and 9-126 or a complementary nucleotide sequence, or as depicted in any one of SEQ ID NOs: 1-4 and 9-126 or of a complementary nucleotide sequence, said box or kit comprising:

a) a pair of nucleotide primers in accordance with the invention, whose hybridization position is located, respectively, on the 5′ side and 3′ side of the target nucleic acid whose amplification is sought; and optionally,

b) reagents necessary for the amplification reaction.

Such an amplification box or kit may comprise at least one pair of nucleotide primers as described above.

The subject of the invention is, in addition, a box or kit for amplifying all or part of a nucleic acid comprising any one of SEQ ID NOs: 1-4 and 9-126 or a complementary nucleotide sequence, said box or kit comprising:

1) a pair of nucleotide primers in accordance with the invention, whose hybridization position is located, respectively, on the 5′ side and 3′ side of the target nucleic acid whose amplification is sought; and optionally,

2) reagents necessary for an amplification reaction.

Such an amplification box or kit may comprise at least one pair of nucleotide primers as described above.

The invention also relates to a box or kit for detecting the presence of a nucleic acid according to the invention in a sample, said box or kit comprising:

a) one or more nucleotide probes according to the invention;

b) where appropriate, reagents necessary for a hybridization reaction.

According to one embodiment, the detection box or kit is characterized in that the nucleotide probe(s) and primer(s)are immobilized on a support.

According to another embodiment, the detection box or kit is characterized in that the nucleotide probe(s) and primer(s) comprise a detectable marker.

According to another embodiment of the detection kit described above, such a kit will comprise a plurality of oligonucleotide probes and/or primers in accordance with the invention that may be used to detect target nucleic acids of interest or, alternatively, to detect mutations in the coding regions and/or the non-coding regions of the nucleic acids according to the invention. The target nucleic acid may comprise a nucleotide sequence of any one of SEQ ID NOs: 1-4 and 9-126 or of a complementary nucleic acid sequence. Alternatively, the target nucleic acid may be a nucleic acid fragment or variant of a nucleic acid comprising any one of SEQ ID NOs: 1-4 and 9-126 or of a complementary nucleotide sequence.

According to the present invention, a primer according to the invention comprises, generally, all or part of any one of SEQ ID NOs: 127-217 or a complementary sequence.

The nucleotide primers according to the invention are particularly useful in methods of genotyping subjects and/or of genotyping populations, in particular in the context of studies of association between particular allele forms or particular forms of groups of alleles (haplotypes) in subjects and the existence of a particular phenotype (character) in these subjects, for example, the predisposition of these subjects to develop diseases linked to a deficiency of cholesterol reverse transport and inflammation signaling lipids or, alternatively, the predisposition of these subjects to develop a pathology whose candidate chromosomal region is situated on chromosome 17, more precisely on the 17q arm and, still more precisely, in the 17q24 locus.

Recombinant Vectors

The invention also relates to a recombinant vector comprising a nucleic acid according to the invention. “Vector” for the purposes of the present invention will be understood to mean a circular or linear DNA or RNA molecule that is either in single-stranded or double-stranded form.

A recombinant vector may comprise a nucleic acid chosen from the following nucleic acids:

a) a nucleic acid comprising a nucleotide sequence of any one of SEQ ID NOs: 1-4 and 9-126 or of a complementary nucleotide sequence,

b) a nucleic acid comprising a nucleotide sequence as depicted in any one of SEQ ID NOs: 1-4 and 9-126 or of a complementary nucleotide sequence,

c) a nucleic acid having at least eight consecutive nucleotides of a nucleic acid comprising a nucleotide sequence of any one of SEQ ID NOs: 1-4 and 9-126 or of a complementary nucleotide sequence;

d) a nucleic acid having at least 80% nucleotide identity with a nucleic acid comprising a nucleotide sequence of any one of SEQ ID NOs: 1-4 and 9-126 or a complementary nucleotide sequence;

e) a nucleic acid having at least 85%, at least 90%, at least 95%, or at least 98% nucleotide identity with a nucleic acid comprising a nucleotide sequence of any one of SEQ ID NOs: 1-4 and 9-126 or a complementary nucleotide sequence;

f) a nucleic acid hybridizing, under high stringency hybridization conditions, with a nucleic acid comprising a nucleotide sequence of any one of SEQ ID NOs: 1-4 and 9-126 or a complementary nucleotide sequence;

g) a nucleic acid encoding a polypeptide comprising an amino acid sequence of SEQ ID NOs: 5-8; and

h) a nucleic acid encoding a polypeptide comprising amino acid sequence selected from SEQ ID NOs: 5-8.

According to one embodiment, a recombinant vector according to the invention is used to amplify a nucleic acid inserted therein, following transformation or transfection of a desired cellular host.

According to another embodiment, a recombinant vector according to the invention corresponds to an expression vector comprising, in addition to a nucleic acid in accordance with the invention, a regulatory signal or nucleotide sequence that directs or controls transcription and/or translation of the nucleic acid and its encoded mRNA.

According to another embodiment, a recombinant vector according to the invention may comprise the following components:

(1) an element or signal for regulating the expression of the nucleic acid to be inserted, such as a promoter and/or enhancer sequence;

(2) a nucleotide coding region comprised within the nucleic acid in accordance with the invention to be inserted into such a vector, said coding region being placed in phase with the regulatory element or signal described in (1); and

(3) an appropriate nucleic acid for initiation and termination of transcription of the nucleotide coding region of the nucleic acid described in (2).

In addition, the recombinant vectors according to the invention may include one or more origins for replication in the cellular hosts in which their amplification or their expression is sought, markers or selectable markers.

By way of example, the bacterial promoters may be the LacI or LacZ promoters, the T3 or T7 bacteriophage RNA polymerase promoters, the lambda phage PR or PL promoters.

The promoters for eukaryotic cells comprise the herpes simplex virus (HSV) virus thymidine kinase promoter or, alternatively, the mouse metallothionein-L promoter.

Generally, for the choice of a suitable promoter, persons skilled in the art can refer to the book by Sambrook et al. (1989, Molecular cloning: a laboratory manual. 2ed. Cold Spring Harbor Laboratory, Cold Spring Harbor, N.Y.) cited above or to the techniques described by Fuller et al. (1996, Immunology, In: Current Protocols in Molecular Biology, Ausubel et al.(eds.).

When the expression of the genomic sequence of any one of the ABCA5, ABCA6, ABCA9, and ABCA10 genes is sought, use may be made of the vectors capable of containing large insertion sequences. In one embodiment, bacteriophage vectors like the P1 bacteriophage vectors, such as the vector p158 or the vector p158/neo8 described by Sternberg (1992, Trends Genet., 8:1-16; 1994, Mamm. Genome, 5:397-404), will be used.

The bacterial vectors according to the invention include, for example, the vectors pBR322(ATCC37017) or, alternatively, vectors such as pAA223-3 (Pharmacia, Uppsala, Sweden), and pGEM1 (Promega Biotech, Madison, Wis., United States).

There may also be cited other commercially available vectors, such as the vectors pQE70, pQE60, pQE9 (Qiagen), psiX174, pBluescript SA, pNH8A, pNH16A, pNH18A, pNH46A, pWLNEO, pSV2CAT, pOG44, pXTI, and pSG (Stratagene).

The invention also encompasses vectors of the baculovirus type such as the vector pVL1392/1393 (Pharmingen), which is used to transfect cells of the Sf9 line (ATCC No. CRL 1711) derived from Spodoptera frugiperda.

Vectors according to the invention may also be adenoviral vectors, such as the human adenovirus of type 2 or 5.

A recombinant vector according to the invention may also be a retroviral vector or an adeno-associated vector (AAV). Adeno-associated vectors are, for example, described by Flotte et al. (1992, Am. J. Respir. Cell Mol. Biol., 7:349-356), Samulski et al. (1989, J. Virol., 63:3822-3828), or McLaughlin B A et al. (1996, Am. J. Hum. Genet., 59:561-569).

To allow the expression of a polynucleotide according to the invention, the polynucleotide must be introduced into a host cell. The introduction of a polynucleotide according to the invention into a host cell may be carried out in vitro, according to techniques well known to persons skilled in the art for transforming or transfecting cells, either in primary culture or in the form of cell lines. It is also possible to carry out the introduction of a polynucleotide according to the invention in vivo or ex vivo, for the prevention or treatment of diseases linked to ABC A5, A6, A9 or A10 deficiencies.

To introduce a polynucleotide or vector of the invention into a host cell, a person skilled in the art can use various techniques, such as calcium phosphate coprecipitation (Graham et al., 1973, Virology, 52:456-457 ; Chen et al., 1987, Mol. Cell. Biol., 7: 2745-2752), DEAE Dextran (Gopal, 1985, Mol. Cell. Biol., 5:1188-1190), electroporation (Tur-Kaspa, 1896, Mol. Cell. Biol., 6:716-718 ; Potter et al., 1984, Proc Natl Acad Sci U S A., 81(22):7161-5), direct microinjection (Harland et al., 1985, J. Cell. Biol., 101:1094-1095), liposomes charged with DNA (Nicolau et al., 1982, Methods Enzymol., 149:157-76; Fraley et al., 1979, Proc. Natl. Acad. Sci. USA, 76:3348-3352).

Once the polynucleotide has been introduced into the host cell, it may be stably integrated into the genome of the cell. The intergration may be achieved at a precise site of the genome, by homologous recombination, or it may be random. In some embodiments, the polynucleotide may be stably maintained in the host cell in the form of an episome fragment, the episome comprising sequences allowing the retention and the replication of the latter, either independently or in a synchronized manner with the cell cycle.

According to a specific embodiment, a method of introducing a polynucleotide according to the invention into a host cell, for example, a host cell obtained from a mammal in vivo, comprises a step during which a preparation comprising a pharmaceutically-compatible vector and a “naked” polynucleotide according to the invention, placed under the control of appropriate regulatory sequences, is introduced by local injection at the site of the chosen tissue, for example, myocardial tissue, the “naked” polynucleotide being absorbed by the myocytes of this tissue.

Compositions for use in vitro and in vivo comprising “naked” polynucleotides are, for example, described in PCT Application No. WO 95/11307 (Institut Pasteur, Inserm, University of Ottawa), as well as in the articles by Tacson et al. (1996, Nature Medicine, 2(8):888-892) and Huygen et al. (1996, Nature Medicine, 2(8):893-898).

According to a specific embodiment of the invention, a composition is provided for the in vivo production of any one of ABCA5, ABCA6, ABCA9, and ABCA10 proteins. This composition comprises a polynucleotide encoding the ABCA5, ABCA6, ABCA9, and ABCA10 polypeptides placed under the control of appropriate regulatory sequences in solution in a physiologically-acceptable vector.

The quantity of vector that is injected into the host organism chosen varies according to the site of the injection. As a guide, there may be injected between about 0.1 and about 100 μg of polynucleotide encoding the ABCA5, ABCA6, ABCA9, and ABCA10 proteins into the body of an animal, for example, into a subject likely to develop a disease linked to ABCA5, A6, A9, or A10 deficiencies.

Consequently, the invention also relates to a composition intended for the prevention of or treatment of a patient or subject affected by ABCA5, A6, A9, or A10 deficiencies, comprising a nucleic acid encoding any one of the ABCA5, ABCA6, ABCA9, and ABCA10 proteins in combination with one or more physiologically-compatible excipients.

Such a composition may comprise a nucleic acid comprising a nucleotide sequence of any one of SEQ ID NOs: 1-4, wherein the nucleic acid is placed under the control of an appropriate regulatory element or signal.

The subject of the invention is, in addition, a composition intended for the prevention of or treatment of a patient or a subject affected by an ABCA5, A6, A9 or A10 deficiency, comprising a recombinant vector according to the invention in combination with one or more physiologically-compatible excipients.

The invention also relates to the use of a nucleic acid according to the invention encoding any one of the ABCA5, ABCA6, ABCA9, and ABCA10 proteins for the manufacture of a medicament intended for the prevention of atherosclerosis in various forms or for the treatment of subjects affected by a dysfunction of cholesterol reverse transport or inflammatory liphophilic substances transport.

The invention also relates to the use of a recombinant vector according to the invention comprising a nucleic acid encoding any one of the ABCA5, ABCA6, ABCA9, and ABCA10 proteins for the manufacture of a medicament intended for the prevention of atherosclerosis in various forms or more particularly for the treatment of subjects affected by a dysfunction of cholesterol reverse transport or inflammatory liphophilic substances transport.

The subject of the invention is therefore also a recombinant vector comprising a nucleic acid according to the invention that encodes any one of the ABCA5, ABCA6, ABCA9, and ABCA10 proteins or polypeptides.

The invention also relates to the use of such a recombinant vector for the preparation of a pharmaceutical composition intended for the treatment and/or for the prevention of diseases or conditions associated with a deficiency of cholesterol reverse transport or inflammatory lipophilic substances transport.

The present invention also relates to the use of cells genetically modified ex vivo with a recombinant vector according to the invention and to cells producing a recombinant vector, wherein the cells are implanted in the body to allow a prolonged and effective expression in vivo of at least a biologically active ABCA5, ABCA6, ABCA9, and ABCA10 polypeptides.

Vectors Useful in Methods of Somatic Gene Therapy and Composition Containing Such Vectors

The present invention also relates to a new therapeutic approach for the treatment of pathologies linked to ABCA5, A6, A9, or A10 deficiencies. It provides an advantageous solution to the disadvantages of the prior art by demonstrating the possibility of treating the pathologies of ABCA5, A6, A9, or A10 deficiencies by gene therapy by the transfer and expression in vivo of a gene encoding at least one of the ABCA5, ABCA6, ABCA9, and ABCA10 proteins involved in the transport of lipophilic substances. The invention offers a simple means allowing a specific and effective treatment of related pathologies such as, for example, atherosclerosis, inflammation, cardiovascular diseases, metabolic diseases, and lipophilic substance-related pathologies.

Gene therapy consists in correcting a deficiency or an abnormality (mutation, aberrant expression, and the like) and in bringing about the expression of a protein of therapeutic interest by introducing genetic information into the affected cell or organ. This genetic information may be introduced either ex vivo into a cell extracted from the organ, the modified cell then being reintroduced into the body, or directly in vivo into the appropriate tissue. In this second case, various techniques exist, among which various transfection techniques involving complexes of DNA and DEAE-dextran (Pagano et al. (1967. J. Virol., 1:891), of DNA and nuclear proteins (Kaneda et al., 1989, Science 243:375), of DNA and lipids (Felgner et al., 1987, PNAS 84:7413), the use of liposomes (Fraley et al., 1980, J.Biol.Chem., 255:10431), and the like. More recently, the use of viruses as vectors for the transfer of genes has appeared as a promising alternative to these physical transfection techniques. In this regard, various viruses have been tested for their ability to infect certain cell populations. In particular, the retroviruses (RSV, HMS, MMS, and the like), the herpes simpex viruses (HSV), the adeno-associated viruses, and the adenoviruses may be mentioned.

The present invention therefore also relates to a new therapeutic approach to the treatment of pathologies linked to ABCA5, A6, A9, or A10 deficiencies, which consists of transferring and expressing in vivo genes encoding any one of the ABCA5, ABCA6, ABCA9, and ABCA10 polypeptides. For example, Applicants have now found that it is possible to construct recombinant vectors comprising a nucleic acid encoding at least one of the ABCA5, ABCA6, ABCA9, and ABCA10 proteins, to administer these recombinant vectors in vivo, and that this administration allows a stable and effective expression of at least one of the biologically active ABCA5, ABCA6, ABCA9, and ABCA10 proteins in vivo, with no cytopathological effect.

Adenoviruses are efficient vectors for the transfer and the expression of any one of the ABCA5, ABCA6, ABCA9, and ABCA10 genes. The use of recombinant adenoviruses as vectors makes it possible to obtain sufficiently high levels of expression of these genes to produce the desired therapeutic effect. The use of other viral vectors such as retroviruses or adeno-associated viruses (MV) that allow a stable expression of the gene is part of the invention.

The present invention is thus likely to offer a new approach to the treatment and prevention of ABCA5, A6, A9, and A10 deficiencies.

The subject of the invention is therefore also a defective recombinant virus comprising a nucleic acid according to the invention that encodes at least one of the ABCA5, ABCA6, ABCA9, and ABCA10 proteins or polypeptides involved in the metabolism of lipophilic substances.

The invention also relates to the use of such a defective recombinant virus for the preparation of a composition which may be useful for the treatment and/or for the prevention of ABCA5, A6, A9 or A10 deficiencies.

The present invention also relates to the use of cells genetically modified ex vivo with such a defective recombinant virus according to the invention, and to cells producing a defective recombinant virus, wherein the cells are implanted in the body, to allow a prolonged and effective expression in vivo of at least one biologically active ABCA5, ABCA6, ABCA9, or ABCA10 polypeptide.

The present invention is particularly advantageous because it is possible to induce a controlled expression, without harmful effect, of the ABCA5, ABCA6, ABCA9, and ABCA10 proteins in organs that are not normally involved in the expression of those proteins. In particular, a significant release of the ABCA5, ABCA6, ABCA9, and ABCA10 proteins is obtained by implantation of cells producing vectors of the invention or infected ex vivo with vectors of the invention.

The activity of these ABC protein transporters produced in the context of the present invention may be of the human or animal ABCA5, ABCA6, ABCA9, and ABCA10 type. The nucleotide sequence used in the context of the present invention may be a cDNA, a genomic DNA (gDNA), an RNA (in the case of retroviruses), or a hybrid construct consisting, for example, of a cDNA into which one or more introns (gDNA) has been inserted. It may also involve synthetic or semisynthetic sequences. In one embodiment of the invention, a cDNA or a gDNA is used. The use of a gDNA allows for better expression in human cells.

To allow their incorporation into a viral vector according to the invention, these nucleotide sequences may be modified, for example, by site-directed mutagenesis, for example, for the insertion of appropriate restriction sites. In the context of the present invention, the use of a nucleic sequence encoding any one of the ABCA5, ABCA6, ABCA9, and ABCA10 proteins is contemplated. Moreover, it is possible to use a construct encoding a derivative of any one of the ABCA5, ABCA6, ABCA9, and ABCA10 proteins. A derivative of any one of the ABCA5, ABCA6, ABCA9, and ABCA10 proteins comprises, for example, any sequence obtained by mutation, deletion, and/or addition relative to the native sequence and encoding a product retaining the lipophilic subtances transport activity. These modifications may be made by techniques known to a person skilled in the art (see general molecular biological techniques below). The biological activity of the derivatives thus obtained can then be easily determined, as indicated in the examples of the measurement of the efflux of the substrate from cells. The derivatives for the purposes of the invention may also be obtained by hybridization from nucleic acid libraries using as a probe the native sequence or a fragment thereof. These derivatives are, for example, molecules having a higher affinity for their binding sites, molecules exhibiting greater resistance to proteases, molecules having a higher therapeutic efficacy or fewer side effects, or optionally new biological properties. The derivatives also include the modified DNA sequences allowing improved expression in vivo.

In one embodiment, the present invention relates to a defective recombinant virus comprising a cDNA encoding any one of the ABCA5, ABCA6, ABCA9, and ABCA10 polypeptides. In another embodiment of the invention, a defective recombinant virus comprises a genomic DNA (gDNA) encoding any one of the ABCA5, ABCA6, ABCA9, and ABCA10 polypeptides. The ABCA5, ABCA6, ABCA9, and ABCA10 polypeptides may comprise an amino acid sequence selected from SEQ ID NOs: 5-8, respectively.

The vectors of the invention may be prepared from various types of viruses. For example, vectors derived from adenoviruses, adeno-associated viruses (MV), herpesviruses (HSV) or retroviruses may be used. An adenovirus may be used for direct administration or for the ex vivo modification of cells intended to be implanted. Alternatively, a retrovirus may be used for the implantation of producing cells.

The viruses according to the invention are usually defective, that is to say that they are incapable of autonomously replicating in the target cell. Generally, the genome of the defective viruses used in the context of the present invention lacks at least the sequences necessary for the replication of said virus in the infected cell. These regions may be either eliminated (completely or partially), made nonfunctional, or substituted with other sequences, for example, with the nucleic sequence encoding any one of the ABCA5, ABCA6, ABCA9, and ABCA10 proteins. The defective virus may retain, however, the sequences of its genome that are necessary for the encapsidation of the viral particles.

With regard to adenoviruses, various serotypes, whose structure and properties vary somewhat, have been characterized, for example, human adenoviruses of type 2 or 5 (Ad 2 or Ad 5) and adenoviruses of animal origin (see Application WO 94/26914). Among the adenoviruses of animal origin that can be used in the context of the present invention, there may be mentioned adenoviruses of canine, bovine, murine (example: Mav1, Beard et al., Virology 75 (1990) 81), ovine, porcine, avian or simian (example: SAV) origin. Preferably, the adenovirus of animal origin is a canine adenovirus, for example, a CAV2 adenovirus [Manhattan or A26/61 strain (ATCC VR-800) for example]. Adenoviruses of human or canine or mixed origin may be used in the context of the invention. In general, the defective adenoviruses of the invention comprise the ITRs, a sequence allowing encapsidation, and a sequence encoding any one of the ABCA5, ABCA6, ABCA9, and ABCA10 proteins. In general, in the genome of the adenoviruses of the invention, the at least E1 region is made nonfunctional. In addition, in the genome of the adenoviruses of the invention, at least one of the E2, E4 and LI -L5 genes may also be nonfunctional. These viral genes may be made nonfunctional by any technique known to a person skilled in the art, for example, by total suppression, by substitution, by partial deletion, or by addition of one or more bases in the inactivated gene(s). Such modifications may be obtained in vitro (on the isolated DNA) or in situ, for example, by means of genetic engineering techniques or by treatment with mutagenic agents. Other regions of the virus also may be modified, for example, the E3 (WO95/02697), E2 (WO94/28938), E4 (WO94/28152, WO94/12649, WO95/02697) and L5 (WO95/02697) regions. According to one embodiment, the adenovirus according to the invention comprises a deletion in the E1 and E4 regions and the sequence encoding any one of the ABCA5, ABCA6, ABCA9, and ABCA10 proteins is inserted at the site of the inactivated E1 region. According to another embodiment, the virus comprises a deletion in the E1 region at the site of which the E4 region and the sequence encoding any one of the ABCA5, ABCA6, ABCA9, and ABCA10 proteins (French Patent Application FR94 13355) is inserted.

The defective recombinant adenoviruses according to the invention may be prepared by any technique known to persons skilled in the art (Levrero et al., 1991 Gene 101; EP 185 573; and Graham, 1984, EMBO J., 3:2917). For example, they may be prepared by homologous recombination between an adenovirus and a plasmid carrying, inter alia, the nucleic acid encoding any one of the ABCA5, ABCA6, ABCA9, and ABCA10 proteins. The homologous recombination occurs after cotransfection of said adenoviruses and plasmid into an appropriate cell line. The cell line used must (i) be transformable by said elements and (ii) contain the sequences required to complement the part of the defective adenovirus genome, which may be in integrated form in order to avoid the risks of recombination. By way of example of a line, there may be mentioned the human embryonic kidney line 293 (Graham et al., 1977, J. Gen. Virol., 36:59), which contains the left part of the genome of an Ad5 adenovirus (12%) integrated into its genome or lines capable of complementing the E1 and E4 functions as described in particular in Application Nos. WO 94/26914 and WO95/02697.

The adeno-associated viruses (AAV) are DNA viruses of relatively small size, which integrate into the genome of the cells that they infect in a stable and site-specific manner. AAVs are capable of infecting a broad spectrum of cells without causing any effect on cellular growth, morphology or differentiation. Moreover, AAVs do not appear to be involved in pathologies in humans. The genome of AAVs has been cloned, sequenced, and characterized. It comprises about 4700 bases and contains an inverted repeat region (ITR) of about 145 bases at each end, which serves as the replication origin for the virus. The remainder of the genome is divided into 2 essential regions carrying the encapsidation functions: the left hand part of the genome, which contains the rep gene involved in the viral replication and the expression of the viral genes; the right hand part of the genome, which contains the cap gene encoding the virus capsid proteins.

The use of vectors derived from AAVs for the transfer of genes in vitro and in vivo has been described in the literature (see in particular WO 91/18088; WO 93/09239; U.S. Pat. Nos. 4,797,368, 5,139,941, EP 488 528). These applications describe various constructs derived from AAVs in which the rep and/or cap genes are deleted and replaced by a gene of interest and their use for transferring in vitro (cells in culture) or in vivo (directly into an organism) a gene of interest. However, none of these documents either describes or suggests the use of a recombinant MV for the transfer and expression in vivo or ex vivo of any one of the ABCA5, ABCA6, ABCA9, and ABCA10 proteins or the advantages of such a transfer. The defective recombinant AAVs according to the invention may be prepared by cotransfection, into a cell line infected with a human helper virus (for example, an adenovirus), of a plasmid containing the sequence encoding any one of the ABCA5, ABCA6, ABCA9, and ABCA10 proteins bordered by two MV inverted repeat regions (ITR) and of a plasmid carrying the MV encapsidation genes (rep and cap genes). The recombinant MVs produced are then purified by conventional techniques.

The construction of recombinant herpesvirus and retrovirus vectors has been widely described in the literature, for example, in Breakfield et al., (1991, New Biologist, 3:203); EP 453242, EP178220, Bernstein et al. (1985); McCormick, (1985. BioTechnology, 3:689), and the like.

Retroviruses are integrating viruses, which infect dividing cells. The genome of the retroviruses comprises two long terminal repeats (LTRs), an encapsidation sequence, and three protein coding regions (gag, pol, and env). In the recombinant vectors derived from retroviruses, the gag, pol, and env genes are generally deleted, completely or partially, and replaced with a heterologous nucleic acid sequence of interest. These vectors may be produced from various types of retroviruses such as, for example, MoMuLV (“murine moloney leukemia virus”; also called MoMLV), MSV (“murine moloney sarcoma virus”), HaSV (“harvey sarcoma virus”); SNV (“spleen necrosis virus”); RSV (“rous sarcoma virus”) or Friend's virus.

To construct recombinant retroviruses containing a sequence encoding any one of the ABCA5, ABCA6, ABCA9, and ABCA10 proteins according to the invention, a plasmid containing, for example, the LTRs, the encapsidation sequence, and the coding sequence is usually constructed and used to transfect a so-called encapsidation cell line, which is capable of providing in trans the retroviral functions deficient in the plasmid. Generally, the encapsidation lines are capable of expressing the gag, pol, and env genes. Such encapsidation lines have been described in the prior art, for example, the PA317 line (U.S. Pat. No. 4,861,719), the PsiCRIP line (WO 90/02806), and the GP+envAm-12 line (WO 89/07150). Moreover, the recombinant retroviruses may contain modifications at the level of the LTRs in order to suppress transcriptional activity, as well as extended encapsidation sequences, containing a portion of the gag gene (Bender et al., 1987, J. Virol., 61:1639). The recombinant retroviruses produced are then purified by conventional techniques.

In one embodiment of the invention, a defective recombinant adenovirus is used. The advantageous properties of adenoviruses are preferred for the in vivo expression of a protein having a lipophilic subtrate transport activity. The adenoviral vectors according to the invention are particularly preferred for direct administration in vivo of a purified suspension and for the ex vivo transformation of cells, in particular autologous cells, in view of their later implantation. Furthermore, the adenoviral vectors according to the invention exhibit, in addition, considerable advantages, such as their very high infection efficiency making it possible to carry out infections using small volumes of viral suspension.

According to another embodiment of the invention, a line producing retroviral vectors containing the sequence encoding any one of the ABCA5, ABCA6, ABCA9, and ABCA10 proteins is used for implantation in vivo. The lines that can be used to this end are, for example, the PA317 (U.S. Pat. No. 4,861,719), PsiCrip (WO 90/02806), and GP+envAm-12 (U.S. Pat. No. 5,278,056) cells modified so as to allow the production of a retrovirus containing a nucleic sequence encoding any one of the ABCA5, ABCA6, ABCA9, and ABCA10 proteins according to the invention. For example, totipotent stem cells, precursors of blood cell lines, may be collected and isolated from a subject. These cells may then be transfected in culture with a retroviral vector containing the sequence encoding any one of the ABCA5, ABCA6, ABCA9, and ABCA10 proteins under the control of viral promter, a nonviral promter, a promoter specific for macrophages, or under the control of its own promoter. These cells are then reintroduced into the subject. The differentiation of these cells will result in blood cells expressing at least one of the ABCA5, ABCA6, ABCA9, and ABCA10 proteins.

In the vectors of the invention, the sequence encoding any one of the ABCA5, ABCA6, ABCA9, and ABCA10 proteins may be placed under the control of signals allowing its expression in the infected cells. These may be expression signals that are homologous or heterologous, i.e., signals different from those which are naturally responsible for the expression of the ABCA5, ABCA6, ABCA9, and ABCA10 proteins. They may also be, for example, sequences responsible for the expression of other proteins or synthetic sequences. They may be sequences of eukaryotic or viral genes or derived sequences, which stimulate or repress the transcription of a gene in a specific manner, in a nonspecific manner, or in an inducible manner. By way of example, they may be promoter sequences derived from the genome of the cell which it is desired to infect or from the genome of a virus, for example, the promoters of the E1A or major late promoter (MLP) genes of adenoviruses, the cytomegalovirus (CMV) promoter, the RSV-LTR, and the like. Among the eukaryotic promoters, there may also be mentioned the ubiquitous promoters (HPRT, vimentin, α-actin, tubulin, and the like), the promoters of the intermediate filaments (desmin, neurofilaments, keratin, GFAP, and the like), the promoters of therapeutic genes (MDR, CFTR, factor VIII type, and the like), tissue-specific promoters (pyruvate kinase, villin, promoter of the fatty acid binding intestinal protein, promoter of the smooth muscle cell m-actin, promoters specific for the liver; Apo Al, Apo All, human albumin, and the like) or promoters responding to a stimulus (steroid hormone receptor, retinoic acid receptor, and the like). In addition, these expression sequences may be modified by the addition of enhancer or regulatory sequences and the like. Moreover, when the inserted gene does not contain expression sequences, it may be inserted into the genome of the defective virus downstream of such a sequence.

In one embodiment, the invention relates to a defective recombinant virus comprising a nucleic acid encoding any one of the ABCA5, ABCA6, ABCA9, and ABCA10 proteins under the control of a promoter chosen from RSV-LTR or the CMV early promoter.

As indicated above, the present invention also relates to any use of a virus as described above for the preparation of a composition for the treatment and/or prevention of pathologies linked to the transport of lipophilic substances.

The present invention also relates to a composition comprising one or more defective recombinant viruses as described above. These compositions may be formulated for administration by the topical, oral, parenteral, intranasal, intravenous, intramuscular, subcutaneous, intraocular, or transdermal route, and the like. Preferably, the compositions of the invention comprise a pharmaceutically-acceptable vehicle or physiologically-compatible excipient for an injectable formulation, for example for an intravenous injection into the subject's portal vein. These may be, for example, isotonic sterile solutions or dry, for example, freeze-dried, compositions that, upon addition of sterilized water or physiological saline as appropriate, allow the preparation of injectable solutions. Direct injection into the subject's portal vein is preferred because it makes it possible to target the infection at the level of the liver and, thus, to concentrate the therapeutic effect at the level of this organ.

The doses of defective recombinant virus used for the injection may be adjusted as a function of various parameters, for example, as a function of the viral vector, of the mode of administration used, of the relevant pathology or of the desired duration of treatment. In general, the recombinant adenoviruses according to the invention are formulated and administered in the form of doses of between 10⁴ and 10¹⁴ pfu/ml, and preferably 10⁶ to 10¹⁰ pfu/ml. The term “pfu” (plaque forming unit) corresponds to the infectivity of a virus solution and is determined by infecting an appropriate cell culture and measuring, generally after 48 hours, the number of plaques that result from infected cell lysis. The techniques for determining the pfu titer of a viral solution are well documented in the literature.

With regard to retroviruses, the compositions according to the invention may directly contain the producing cells with a view to their implantation.

In this regard, another subject of the invention relates to any mammalian cell infected with one or more defective recombinant viruses according to the invention. For example, the invention encompasses any population of human cells infected with such viruses. These may be cells of blood origin (totipotent stem cells or precursors), fibroblasts, myoblasts, hepatocytes, keratinocytes, smooth muscle and endothelial cells, glial cells, and the like.

The cells according to the invention may be derived from primary cultures. These may be collected by any technique known to persons skilled in the art and then cultured under conditions allowing their proliferation. With regard to fibroblasts, these may be easily obtained from biopsies, for example, according to the technique described by Ham (1980). These cells may be used directly for infection with the viruses or stored, for example, by freezing, for the establishment of autologous libraries in view of a subsequent use. The cells according to the invention may be secondary cultures obtained, for example, from pre-established libraries (see for example EP 228458, EP 289034, EP 400047, EP 456640).

The cells in culture are then infected with a recombinant virus according to the invention in order to confer on them the capacity to produce at least one biologically active ABCA5, ABCA6, ABCA9, and ABCA10 protein. The infection is carried out in vitro according to techniques known to persons skilled in the art. For example, depending on the type of cells used and the desired number of copies of virus per cell, persons skilled in the art can adjust the multiplicity of infection and the number of infectious cycles produced. It is clearly understood that these steps must be carried out under appropriate conditions of sterility when the cells are intended for administration in vivo. The doses of recombinant virus used for the infection of the cells may be adjusted by persons skilled in the art according to the desired aim. The conditions described above for administration in vivo may be adapted to infection in vitro. For infection with a retrovirus, it is also possible to co-culture a cell to be infected with a cell producing the recombinant retrovirus according to the invention. This makes it possible to avoid purifying the retrovirus.

Another subject of the invention relates to an implant comprising mammalian cells infected with one or more defective recombinant viruses according to the invention or cells producing recombinant viruses and an extracellular matrix. Preferably, the implants according to the invention comprise 10⁵ to 10¹⁰ cells. More preferably, they comprise 10⁶ to 10⁸ cells.

In addition to the extracellular matrix, the implants of the invention may comprise a gelling compound and, optionally, a support allowing the anchorage of the cells.

For the preparation of the implants according to the invention, various types of gelling agents may be used. The gelling agents are used for the inclusion of the cells in a matrix having the constitution of a gel and, where appropriate, for promoting the anchorage of the cells on the support. Various cell adhesion agents can therefore be used as gelling agents, such as, for example, collagen, gelatin, glycosaminoglycans, fibronectin, lectins, and the like. Preferably, collagen is used in the context of the present invention. This may be collagen of human, bovine, or murine origin. More preferably, type I collagen is used.

As indicated above, the compositions according to the invention may comprise a support allowing the anchorage of the cells. The term “anchorage” designates any form of biological and/or chemical and/or physical interaction causing the adhesion and/or the attachment of the cells to the support. Moreover, the cells may either cover the support used, penetrate inside this support, or both. It is preferred to use a solid, nontoxic, and/or biocompatible support. For example, it is possible to use polytetrafluoroethylene (PTFE) fibers or a support of biological origin.

The present invention thus offers a very effective means for the treatment or prevention of pathologies linked to the transport of lipophilic substances.

In addition, this treatment may be applied to both humans and any animals such as ovines, bovines, domestic animals (dogs, cats and the like), horses, fish, and the like.

Recombinant Host Cells

The invention relates to a recombinant host cell comprising a nucleic acid of the invention, for example, a nucleic acid comprising a nucleotide sequence selected from SEQ ID NOs: 1-4 and 9-126 or a complementary nucleotide sequence thereof.

The invention also relates to a recombinant host cell comprising a nucleic acid of the invention, for example, a nucleic acid comprising a nucleotide sequence as depicted in SEQ ID NOs: 1-4 and 9-126 or a complementary nucleotide sequence thereof.

According to another aspect, the invention relates to a recombinant host cell comprising a recombinant vector according to the invention. Therefore, the invention also relates to a recombinant host cell comprising a recombinant vector comprising any of the nucleic acids of the invention, for example, a nucleic acid comprising a nucleotide sequence of selected from SEQ ID NOs: 1-4 and 9-126 or a complementary nucleotide sequence thereof.

The invention also relates to a recombinant host cell comprising a recombinant vector comprising a nucleic acid comprising a nucleotide sequence as depicted in any one of SEQ ID NOs: 1-4 and 9-126 or of a complementary nucleotide sequence thereof.

Host cells according to the invention are, for example, the following:

a) prokaryotic host cells: strains of Escherichia coli (strain DH5-α), of Bacillus subtilis, of Salmonella typhimurium, or strains of genera such as Pseudomonas, Streptomyces and Staphylococus; and

b) eukaryotic host cells: HeLa cells (ATCC No. CCL2), Cv 1 cells (ATCC No. CCL70), COS cells (ATCC No. CRL 1650), Sf-9 cells (ATCC No. CRL 1711), CHO cells (ATCC No. CCL-61), or 3T3 cells (ATCC No. CRL-6361).

Methods for Producing ABCA5, ABCA6, ABCA9, and ABCA10 Polypeptides

The invention also relates to a method for the production of a polypeptide comprising an amino acid sequence of any one of SEQ ID NOs: 5-8, said method comprising:

a) inserting a nucleic acid encoding said polypeptide into an appropriate vector;

b) culturing, in an appropriate culture medium under conditions allowing the expression of said polypeptide, a previously transformed host cell or transfecting a host cell with the recombinant vector of step a);

c) recovering the conditioned culture medium or lysing the host cell, for example, by sonication or by osmotic shock;

d) separating and purifying said polypeptide from said culture medium or, alternatively, from the cell lysates obtained in step c); and

e) where appropriate, characterizing the recombinant polypeptide produced.

The polypeptides according to the invention may be characterized by binding to an immunoaffinity chromatography column on which the antibodies directed against this polypeptide or against a fragment or a variant thereof have been previously immobilized.

According to another aspect, a recombinant polypeptide according to the invention may be purified by passing it over an appropriate series of chromatography columns, according to methods known to persons skilled in the art and described for example in F. Ausubel et al (1989, Current Protocols in Molecular Biology, Green Publishing Associates and Wiley Interscience, N.Y).

A polypeptide according to the invention may also be prepared by conventional chemical synthesis techniques either in homogeneous solution or in solid phase. By way of illustration, a polypeptide according to the invention may be prepared by the technique either in homogeneous solution described by Houben Weyl (1974, Methode der Organischen Chemie, E. Wunsch Ed., 15-I:15-II) or the solid phase synthesis technique described by Merrifield (1965, Nature, 207(996):522-523; 1965, Science, 150(693):178-185).

A polypeptide termed “homologous” to a polypeptide having an amino acid sequence selected from SEQ ID NOs: 5-8 also forms part of the invention. Such a homologous polypeptide comprises an amino acid sequence possessing one or more substitutions of an amino acid by an equivalent amino acid of SEQ ID NOs:5-8.

An “equivalent amino acid” according to the present invention will be understood to mean, for example, replacement of a residue in the L form by a residue in the D form or the replacement of a glutamic acid (E) by a pyro-glutamic acid according to techniques well known to persons skilled in the art. By way of illustration, the synthesis of a peptide containing at least one residue in the D form is described by Koch (1977). According to another aspect, two amino acids belonging to the same class, that is to say two uncharged polar, nonpolar, basic or acidic amino acids, are also considered as equivalent amino acids.

Polypeptides comprising at least one nonpeptide bond such as a retro-inverse bond (NHCO), a carba bond (CH₂CH₂), or a ketomethylene bond (CO—CH₂) also form part of the invention.

The polypeptides according to the invention comprising one or more additions, deletions, substitutions of at least one amino acid generally retain their capacity to be recognized by antibodies directed against the nonmodified polypeptides.

Antibodies

The ABCA5, ABCA6, ABCA9, and ABCA10 polypeptides according to the invention, for example, 1) a polypeptide comprising an amino acid sequence of any one of SEQ ID NOs: 5-8, 2) a polypeptide fragment or variant of a polypeptide comprising an amino acid sequence of any one of SEQ ID NOs: 5-8, or 3) a polypeptide termed “homologous” to a polypeptide comprising amino acid sequence selected from SEQ ID NOs: 5-8, may be used for the preparation of an antibody, which may be useful, for example, for detecting the production of a normal or altered form of the ABCA5, ABCA6, ABCA9, and ABCA10 polypeptides in a patient.

An antibody directed against a polypeptide termed “homologous” to a polypeptide having an amino acid sequence selected from SEQ ID NOs: 5-8 also forms part of the invention. Such an antibody is directed against a homologous polypeptide comprising an amino acid sequence possessing one or more substitutions of an amino acid by an equivalent amino acid of SEQ ID NOs: 5-8.

“Antibody” for the purposes of the present invention will be understood to mean in particular polyclonal or monoclonal antibodies or fragments (for example the F(ab)′₂ and Fab fragments) or any polypeptide comprising a domain of the initial antibody recognizing the target polypeptide or polypeptide fragment according to the invention.

Monoclonal antibodies may be prepared from hybridomas according to the technique described by Kohler and Milstein (1975, Nature, 256:495-497).

According to the invention, a polypeptide produced recombinantly or by chemical synthesis, and fragments or other derivatives or analogs thereof, including fusion proteins, may be used as an immunogen to generate antibodies that recognize a polypeptide according to the invention. Such antibodies include but are not limited to polyclonal, monoclonal, chimeric, single chain, Fab fragments, and an Fab expression library. The anti-ABCA5, anti-ABCA6, anti-ABCA9, and anti-ABCA10 antibodies of the invention may be cross reactive, e.g., they may recognize corresponding ABCA5, ABCA6, ABCA9, and ABCA10 polypeptides from different species. Polyclonal antibodies have greater likelihood of cross reactivity. Alternatively, an antibody of the invention may be specific for a single form of any one of ABCA5, ABCA6, ABCA9, and ABCA10. Preferably, such an antibody is specific for any one of human ABCA5, ABCA6, ABCA9, and ABCA10.

Various procedures known in the art may be used forthe production of polyclonal antibodies to any one of the ABCA5, ABCA6, ABCA9, and ABCA10 polypeptides or derivatives or analogs thereof. For the production of antibody, various host animals can be immunized by injection with any one of the ABCA5, ABCA6, ABCA9, and ABCA10 polypeptides or a derivatives (e.g., a fragment or fusion protein) thereof, including, but not limited to, rabbits, mice, rats, sheep, goats, etc. In one embodiment, any one of the ABCA5, ABCA6, ABCA9, and ABCA10 polypeptides or fragments thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete or incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For the preparation of monoclonal antibodies directed toward any one of the ABCA5, ABCA6, ABCA9, and ABCA10 polypeptides or fragments, analogs, or derivatives thereof, any technique that provides for the production of antibody molecules by continuous cell lines in culture may be used. These include, but are not limited to, the hybridoma technique originally developed by Kohler and Milstein (1975, Nature, 256:495-497), as well as the trioma technique, the human B-cell hybridoma technique (Kozbor et al., 1983, Immunology Today, 4:72; Cote et al. 1983, Proc. Natl. Acad. Sci. U.S.A. 80:2026-2030), and the EBV-hybridoma technique to produce human monoclonal antibodies (Cole et al., 1985, In: Monoclonal Antibodies and Cancer Therapy, Alan R. Liss, Inc., pp. 77-96). In an additional embodiment of the invention, monoclonal antibodies can be produced in germ-free animals (WO 89/12690). In fact, according to the invention, techniques developed for the production of “chimeric antibodies” (Morrison et al., 1984, J. Bacteriol. 159:870; Neuberger et al., 1984, Nature, 312:604-608; Takeda et al., 1985, Nature 314:452-454) by splicing the genes from a mouse antibody molecule specific for any one of ABCA5, ABCA6, ABCA9, and ABCA10 polypeptides together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, such as an allergic response.

According to the invention, techniques described for the production of single chain antibodies (U.S. Pat. Nos. 5,476,786 and 5,132,405 to Huston; U.S. Pat. No. 4,946,778) can be adapted to produce ABCA5, ABCA6, ABCA9, and ABCA10 polypeptide-specific single chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for any one of the ABCA5, ABCA6, ABCA9, and ABCA10 polypeptides, its derivatives, or analogs.

Antibody fragments that contain the idiotype of the antibody molecule can be generated by known techniques. For example, such fragments include, but are not limited to, the F(ab′)₂ fragment, which can be produced by pepsin digestion of the antibody molecule; the Fab′ fragments, which can be generated by reducing the disulfide bridges of the F(ab′)₂ fragment, and the Fab fragments, which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme, or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labelled. Many means are known in the art for detecting binding in an immunoassay and are within the scope of the present invention. For example, to select antibodies that recognize a specific epitope of any one of the ABCA5, ABCA6, ABCA9, and ABCA10 polypeptides, one may assay hybridomas for a product that binds to any one of the ABCA5, ABCA6, ABCA9, and ABCA10 polypeptide fragments containing such epitope. For selection of an antibody specific to any one of the ABCA5, ABCA6, ABCA9, and ABCA10 polypeptides from a particular species of animal, one can select on the basis of positive binding with any one of the ABCA5, ABCA6, ABCA9, and ABCA10 polypeptides expressed by or isolated from cells of that species of animal.

The foregoing antibodies can be used in methods known in the art relating to the localization and activity of any one of the ABCA5, ABCA6, ABCA9, and ABCA10 polypeptides, e.g., for western blotting, ABCA5, ABCA6, ABCA9, and ABCA10 polypeptides in situ, measuring levels thereof in appropriate physiological samples, etc. using any of the detection techniques mentioned above or known in the art.

In a specific embodiment, antibodies that agonize or antagonize the activity of any one of the ABCA5, ABCA6, ABCA9, and ABCA10 polypeptides can be generated. Such antibodies can be tested using the assays described infra for identifying ligands.

The present invention relates to an antibody directed against 1) a polypeptide comprising an amino acid sequence of any one of SEQ ID NOs: 5-8, 2) a polypeptide fragment or variant of a polypeptide comprising an amino acid sequence of any one of SEQ ID NOs: 5-8, or 3) a polypeptide termed “homologous” to a polypeptide comprising amino acid sequence selected from SEQ ID NOS:5-8, also forms part of the invention, as produced in the trioma technique or the hybridoma technique described by Kozbor et al. (1983, Hybridoma, 2(1):7-16).

The invention also relates to single-chain Fv antibody fragments (ScFv) as described in U.S. Pat. No. 4,946,778 or by Martineau et al. (1998, J Mol Biol, 280(1):117-127).

The antibodies according to the invention also comprise antibody fragments obtained with the aid of phage libraries as described by Ridder et al., (1995, Biotechnology (NY), 13(3):255-260) or humanized antibodies as described by Reinmann et al. (1997, AIDS Res Hum Retroviruses, 13(11):933-943) and Leger et al., (1997, Hum Antibodies, 8(1):3-16).

The antibody preparations according to the invention are useful in immunological detection tests intended for the identification of the presence and/or of the quantity of antigens present in a sample.

An antibody according to the invention may comprise, in addition, a detectable marker that is isotopic or nonisotopic, for example, fluorescent, or may be coupled to a molecule such as biotin according to techniques well known to persons skilled in the art.

Thus, the subject of the invention is, in addition, a method of detecting the presence of a polypeptide according to the invention in a sample, said method comprising:

a) bringing the sample to be tested into contact with an antibody directed against 1) a polypeptide. comprising an amino acid sequence of any one of SEQ ID NOs: 5-8, 2) a polypeptide fragment or variant of a polypeptide comprising an amino acid sequence of any one of SEQ ID NOs: 5-8, or 3) a polypeptide termed “homologous” to a polypeptide comprising amino acid sequence selected from SEQ ID NOs: 5-8, and

b) detecting the antigen/antibody complex formed.

The invention also relates to a box or kit for diagnosis or for detecting the presence of a polypeptide in accordance with the invention in a sample, said box comprising:

a) an antibody directed against 1) a polypeptide comprising an amino acid sequence of any one of SEQ ID NOs:5-8, 2) a polypeptide fragment or variant of a polypeptide comprising an amino acid sequence of any one of SEQ ID NOs: 5-8, or 3) a polypeptide termed “homologous” to a polypeptide comprising amino acid sequence selected from SEQ ID NOs: 5-8, and

b) a reagent allowing the detection of the antigen/antibody complexes formed.

Compositions and Therapeutic Methods of Treatment

The invention also relates to compositions intended for the prevention and/or treatment of a deficiency in the transport of cholesterol or inflammatory lipid substances, characterized in that they comprise a therapeutically effective quantity of a polynucleotide capable of giving rise to the production of an effective quantity of at least one of the functional ABCA5, ABCA6, ABCA9, and ABCA10 polypeptides, for example, a polypeptide comprising an amino acid sequence of SEQ ID NOs: 5-8.

The invention also provides compositions comprising a nucleic acid encoding any one of the ABCA5, ABCA6, ABCA9, and ABCA10 polypeptides according to the invention and compositions comprising any one of the ABCA5, ABCA6, ABCA9, and ABCA10 polypeptides according to the invention intended for the prevention and/or treatment of diseases linked to a deficiency in the transport of cholesterol or inflammatory lipid substances.

The present invention also relates to a new therapeutic approach for the treatment of pathologies linked to the transport of lipophilic substances, comprising transferring and expressing in vivo nucleic acids encoding at least one of ABCA5, ABCA6, ABCA9, and ABCA10 proteins according to the invention.

Thus, the present invention offers a new approach for the treatment and/or the prevention of pathologies linked to abnormalities of the transport of lipophilic substances.

Consequently, the invention also relates to a composition intended for the prevention of or treatment of subjects affected by a dysfunction in lipophilic substances, comprising a nucleic acid encoding at least one of the ABCA5, ABCA6, ABCA9, and ABCA10 proteins in combination with one or more physiologically-compatible vehicles and/or excipients.

According to a specific embodiment of the invention, a composition is provided for the in vivo production of at least one of the ABCA5, ABCA6, ABCA9, and ABCA10 proteins. This composition comprises a nucleic acid encoding any one of the ABCA5, ABCA6, ABCA9, and ABCA10 polypeptides placed under the control of appropriate regulatory sequences in solution in a physiologically-acceptable vehicle and/or excipient.

Therefore, the present invention also relates to a composition comprising a nucleic acid encoding a polypeptide comprising an amino acid sequence of SEQ ID NOs: 5-8, wherein the nucleic acid is placed under the control of appropriate regulatory elements. Such a composition may comprise a nucleic acid comprising a nucleotide sequence of SEQ ID NOs:1-4, placed under the control of appropriate regulatory elements.

According to another aspect, the subject of the invention is also a preventive and/or curative therapeutic method of treating diseases caused by a deficiency in the transport of lipophilic substances, such a method comprising administering to a patient a nucleic acid encoding any one of the ABCA5, ABCA6, ABCA9, and ABCA10 polypeptides according to the invention in said patient, said nucleic acid being, where appropriate, combined with one or more physiologically-compatible vehicles and/or excipients.

The invention also relates to a composition intended for the prevention of or treatment of subjects affected by a dysfunction in the transport of lipophilic substances, comprising a recombinant vector according to the invention in combination with one or more physiologically-compatible excipients.

According to one embodiment, a method of introducing a nucleic acid according to the invention into a host cell, for example, a host cell obtained from a mammal, in vivo, comprises a step during which a preparation comprising a pharmaceutically-compatible vector and a “naked” nucleic acid according to the invention, placed under the control of appropriate regulatory sequences, is introduced by local injection at the site of the chosen tissue, for example, a smooth muscle tissue, the “naked” nucleic acid being absorbed by the cells of this tissue.

The invention also relates to the use of a nucleic acid according to the invention, encoding any one of the ABCA5, ABCA6, ABCA9, and ABCA10 proteins, for the manufacture of a medicament intended for the prevention and/or treatment in various forms or more particularly for the treatment of subjects affected by a dysfunction in the transport of lipophilic substances.

The invention also relates to the use of a recombinant vector according to the invention, comprising a nucleic acid encoding any one of the ABCA5, ABCA6, ABCA9, and ABCA10 proteins, for the manufacture of a medicament intended for the prevention and/or treatment of subjects affected by a dysfunction in the transport of lipophilic substances.

As indicated above, the present invention also relates to the use of a defective recombinant virus according to the invention for the preparation of a composition for the treatment and/or prevention of pathologies linked to the transport of lipophilic substances.

The invention relates to the use of such a defective recombinant virus for the preparation of a composition intended for the treatment and/or prevention of a deficiency associated with the transport of lipophilic substances. Thus, the present invention also relates to a composition comprising one or more defective recombinant viruses according to the invention.

The present invention also relates to the use of cells genetically modified ex vivo with a virus according to the invention and to producing cells such viruses, implanted in the body, allowing a prolonged and effective expression in vivo of at least one biologically active ABCA5, ABCA6, ABCA9, and ABCA10 protein.

The present invention shows that it is possible to incorporate a nucleic acid encoding any one of the ABCA5, ABCA6, ABCA9, and ABCA10 polypeptides into a viral vector, and that these vectors make it possible to effectively express a biologically active, mature form of the encoded protein. More particularly, the invention shows that the in vivo expression of any one of the ABCA5, ABCA6, ABCA9, and ABCA10 genes may be obtained by direct administration of an adenovirus or by implantation of a producing cell or of a cell genetically modified by an adenovirus or by a retrovirus incorporating such a DNA.

The compositions of the invention may comprise a pharmaceutically-acceptable vehicle or physiologically-compatible excipient for an injectable formulation, for example, for an intravenous injection into the subject's portal vein. These may be, for example, isotonic sterile solutions or dry, for example, freeze-dried, compositions which, upon addition of sterilized water or physiological saline, as appropriate, allow the preparation of injectable solutions. Direct injection into the subject's portal vein is preferred because it makes it possible to target the infection at the level of the liver and, thus, to concentrate the therapeutic effect at the level of this organ.

The term “pharmaceutically-acceptable vehicle or excipient” includes diluents and fillers that are pharmaceutically-acceptable for method of administration, are sterile, and may be aqueous or oleaginous suspensions formulated using suitable dispersing or wetting agents and suspending agents. The particular pharmaceutically-acceptable carrier and the ratio of active compound to carrier are determined by the solubility and chemical properties of the composition, the particular mode of administration, and standard pharmaceutical practice.

Any nucleic acid, polypeptide, vector, or host cell of the invention may be introduced in vivo in a pharmaceutically-acceptable vehicle or excipient. The phrase “pharmaceutically-acceptable” refers to molecular entities and compositions that are physiologically-tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and, more particularly, in humans. The term “excipient” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, and the like. Water or aqueous solution saline solutions and aqueous dextrose and glycerol solutions may be employed as excipients, particularly for injectable solutions. Suitable pharmaceutical excipients are described in “Remington's Pharmaceutical Sciences” by E. W. Martin.

The pharmaceutical compositions according to the invention may be equally well administered by the oral, rectal, parenteral, intravenous, subcutaneous, or intradermal route.

According to another aspect, the subject of the invention is also a preventive and/or curative therapeutic method of treating diseases caused by a deficiency in the transport of cholesterol or inflammatory lipid substances, comprising administering to a patient or subject a nucleic acid encoding any one of the ABCA5, ABCA6, ABCA9, and ABCA10 polypeptides, said nucleic acid being combined with one or more physiologically-compatible vehicles and/or excipients.

In another embodiment, the nucleic acids, recombinant vectors, and compositions according to the invention can be delivered in a vesicle, in particular a liposome (see Langer, 1990, Science, 249:1527-1533; Treat et al., 1989, Liposomes in the Therapy of Infectious Disease and Cancer, Lopez-Berestein and Fidler (eds.), Liss: New York, pp. 353-365; and Lopez-Berestein, 1989, In: Liposomes in the Therapy of Infectious Disease and Cancer, Lopez-Berestein and Fidler (eds.), Liss: New York, pp. 317-327).

In a further aspect, recombinant cells that have been transformed with a nucleic acid according to the invention and that express high levels of any one of ABCA5, ABCA6, ABCA9, and ABCA10 polypeptides according to the invention can be transplanted in a subject in need of any one of ABCA5, ABCA6, ABCA9, and ABCA10 polypeptides. Preferably autologous cells transformed with an any one of ABCA5, ABCA6, ABCA9, and ABCA10 encoding nucleic acids according to the invention are transplanted to avoid rejection; alternatively, technology is available to shield non-autologous cells that produce soluble factors within a polymer matrix that prevents immune recognition and rejection.

A subject in whom administration of the nucleic acids, polypeptides, recombinant vectors, recombinant host cells, and compositions according to the invention is performed is preferably a human, but can be any animal. Thus, as can be readily appreciated by one of ordinary skill in the art, the methods and pharmaceutical compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

Preferably, a pharmaceutical composition comprising a nucleic acid, a recombinant vector, or a recombinant host cell, as defined above, will be administered to the patient or subject.

Mehtods of Screening an Agonist or Antagonist Compound for the ABCA5, ABCA6, ABCA9, and ABCA10 Polypeptides

According to another aspect, the invention also relates to various methods of screening compounds or small molecules for therapeutic use which are useful in the treatment of diseases due to a deficiency in the transport of cholesterol or inflammatory lipid substances.

The invention therefore also relates to the use of any one of ABCA5, ABCA6, ABCA9, and ABCA10 polypeptides, or of cells expressing any one of ABCA5, ABCA6, ABCA9, and ABCA10 polypeptides, for screening active ingredients for the prevention and/or treatment of diseases resulting from a dysfunction in ABCA5, ABCA6, ABCA9, or ABCA10 defiencies. The catalytic sites and oligopeptide or immunogenic fragments of any one of ABCA5, ABCA6, ABCA9, and ABCA10 polypeptides can serve for screening product libraries by a whole range of existing techniques. The polypeptide fragment used in this type of screening may be free in solution, bound to a solid support, at the cell surface or in the cell. The formation of the binding complexes between any one of the ABCA5, ABCA6, ABCA9, and ABCA10 polypeptide fragments and the tested agent can then be measured.

Another product screening technique which may be used in high-flux screenings giving access to products having affinity for the protein of interest is described in application WO84/03564. In this method, applied to any one of ABCA5, ABCA6, ABCA9, and ABCA10 proteins, various products are synthesized on a solid surface. These products react with corresponding ABCA5, ABCA6, ABCA9, and ABCA10 proteins or fragments thereof and the complex is washed. The products binding any one of the ABCA5, ABCA6, ABCA9, and ABCA10 proteins are then detected by methods known to persons skilled in the art. Non-neutralizing antibodies can also be used to capture a peptide and immobilize it on a support.

Another possibility is to perform a product screening method using any one of the ABCA5, ABCA6, ABCA9, and ABCA10 neutralizing competition antibodies, at least one of ABCA5, ABCA6, ABCA9, and ABCA10 proteins and a product potentially binding any one of the ABCA5, ABCA6, ABCA9, and ABCA10 proteins. In this manner, the antibodies may be used to detect the presence of a peptide having a common antigenic unit with any one of ABCA5, ABCA6, ABCA9, and ABCA10 polypeptides or proteins.

Of the products to be evaluated for their ability to increase activity of any one of ABCA5, ABCA6, ABCA9, and ABCA10, there may be mentioned in particular kinase-specific ATP homologs involved in the activation of the molecules, as well as phosphatases, which may be able to avoid the dephosphorylation resulting from said kinases. There may be mentioned in particular inhibitors of the phosphodiesterase (PDE) theophylline and 3-isobutyl-1-methylxanthine type or the adenylcyclase forskolin activators.

Accordingly, this invention relates to the use of any method of screening products, i.e., compounds, small molecules, and the like, based on the method of translocation of cholesterol or lipophilic substances between the membranes or vesicles, this being in all synthetic or cellular types, that is to say of mammals, insects, bacteria, or yeasts expressing constitutively or having incorporated any one of human ABCA5, ABCA6, ABCA9, and ABCA10 encoding nucleic acids. To this effect, labeled lipophilic substances analogs may be used.

Furthermore, knowing that the disruption of numerous transporters have been described (van den Hazel et al., 1999, J. Biol Chem, 274: 1934-41), it is possible to think of using cellular mutants having a characteristic phenotype and to complement the function thereof with at least one of ABCA5, ABCA6, ABCA9, and ABCA10 proteins and to use the whole for screening purposes.

The invention also relates to a method of screening a compound or small molecule active on the transport of lipophilic substances, an agonist or antagonist of any one of ABCA5, ABCA6, ABCA9, and ABCA10 polypeptides, said method comprising the following steps:

a) preparing a membrane vesicle comprising at least one of the ABCA5, ABCA6, ABCA9, and ABCA10 polypeptides and a lipid substrate comprising a detectable marker;

b) incubating the vesicle obtained in step a) with an agonist or antagonist candidate compound;

c) qualitatively and/or quantitatively measuring release of the lipid substrate comprising a detectable marker; and

d) comparing the release measurement obtained in step b) with a measurement of release of labeled lipophilic substrate by a vesicle that has not been previously incubated with the agonist or antagonist candidate compound.

ABCA5, ABCA6, ABCA9, and ABCA10 polypeptides comprise an amino acid sequence selected from SEQ ID NOs: 5-8.

According to a first aspect of the above screening method, the membrane vesicle is a synthetic lipid vesicle, which may be prepared according to techniques well known to a person skilled in the art. According to this particular aspect, ABCA5, ABCA6, ABCA9, and ABCA10 proteins may be recombinant proteins.

According to a second aspect, the membrane vesicle is a vesicle of a plasma membrane derived from cells expressing at least one of ABCA5, ABCA6, ABCA9, and ABCA10 polypeptides. These may be cells naturally expressing any one of ABCA5, ABCA6, ABCA9, and ABCA10 polypeptides or cells transfected with a nucleic acid encoding at least one ABCA5, ABCA6, ABCA9, and ABCA10 polypeptides or recombinant vector comprising a nucleic acid encoding any one of ABCA5, ABCA6, ABCA9, and ABCA10 polypeptides.

According to a third aspect of the above screening method, the lipid substrate is chosen from prostaglandins or prostacyclins.

According to a fourth aspect of the above screening method, the lipid substrate is chosen from cholesterol or phosphatidylcholine.

According to a fifth aspect, the lipid substrate is radioactively labelled, for example with an isotope chosen from ³H or ¹²⁵I.

According to a sixth aspect, the lipid substrate is labelled with a fluorescent compound, such as NBD or pyrene.

According to a seventh aspect, the membrane vesicle comprising the labelled lipophilic substances and any one of the ABCA5, ABCA6, ABCA9, and ABCA10 polypeptides is immobilized at the surface of a solid support prior to step b).

According to a eighth aspect, the measurement of the fluorescence or of the radioactivity released by the vesicle is the direct reflection of the activity of lipid substrate transport by any one of the ABCA5, ABCA6, ABCA9, and ABCA10 polypeptides.

The invention also relates to a method of screening a compound or small molecule active on the transport of cholesterol or lipid substances, an agonist or antagonist of any one of ABCA5, ABCA6, ABCA9, and ABCA10 polypeptides, said method comprising the following steps:

a) obtaining cells, for example a cell line, that, either naturally or after transfecting the cell with any one of ABCA5, ABCA6, ABCA9, AND ABCA10 encoding nucleic acids, expresses any one of ABCA5, ABCA6, ABCA9, AND ABCA10 polypeptides;

b) incubating the cells of step a) in the presence of an anion labelled with a detectable marker;

c) washing the cells of step b) in order to remove the excess of the labelled anion which has not penetrated into these cells;

d) incubating the cells obtained in step c) with an agonist or antagonist candidate compound for any one of ABCA5, ABCA6, ABCA9, and ABCA10 polypeptides;

e) measuring efflux of the labelled anion; and

f) comparing the value of efflux of the labelled anion determined in step e) with a value of the efflux of a labelled anion measured with cells that have not been previously incubated in the presence of the agonist or antagonist candidate compound of any one of ABCA5, ABCA6, ABCA9, and ABCA10 polypeptides.

In a first specific embodiment, any one of the ABCA5, ABCA6, ABCA9, and ABCA10 polypeptides comprise an amino acid sequence of SEQ ID NOs: 5-8.

According to a second aspect, the cells used in the screening method described above may be cells not naturally expressing, or alternatively expressing at a low level, any one of the ABCA5, ABCA6, ABCA9, and ABCA10 polypeptides, said cells being transfected with a recombinant vector according to the invention capable of directing the expression of a nucleic acid encoding any one of the ABCA5, ABCA6, ABCA9, and ABCA10 polypeptides.

According to a third aspect, the cells may be cells having a natural deficiency in anion transport, or cells pretreated with one or more anion channel inhibitors such as Verapamil™ or tetraethylammonium.

According to a fourth aspect of said screening method, the anion is a radioactively labelled iodide, such as the salts K¹²⁵I or Na¹²⁵I.

According to a fifth aspect, the measurement of efflux of the labelled anion is determined periodically over time during the experiment, thus making it possible to also establish a kinetic measurement of this efflux.

According to a sixth aspect, the value of efflux of the labelled anion is determined by measuring the quantity of labelled anion present at a given time in the cell culture supernatant.

According to a seventh aspect, the value of efflux of the labelled anion is determined as the proportion of radioactivity found in the cell culture supernatant relative to the total radioactivity corresponding to the sum of the radioactivity found in the cell lysate and the radioactivity found in the cell culture supernatant.

The subject of the invention is also a method of screening a compound or small molecule active on the metabolism of lipophilic substances, an agonist or antagonist of any one of ABCA5, ABCA6, ABCA9, and ABCA10 polypeptides, said method comprising the following steps:

a) culturing cells of a human myocyte line in an appropriate culture medium, in the presence of purified human albumin;

b) incubating the cells of step a) simultaneously in the presence of a compound stimulating the production of interleukine and of an agonist or antagonist candidate compound;

c) incubating the cells obtained in step b) in the presence of an appropriate concentration of ATP;

d) measuring interleukinereleased into the cell culture supernatant; and

e) comparing the value of the release of the interleukineobtained in step d) with the value of the interleukinereleased into the culture supernatant of cells which have not been previously incubated in the presence of the agonist or antagonist candidate compound.

According to a first aspect of the screening method described above, the cells used belong to the human or mousemyocytes.

According to a second aspect of the screening method, the compound stimulating the production of interleukineis a lipopolysaccharide.

According to a third aspect of said method, the production of all interleukinesand TNF alpha by these cells is also qualitatively and/or quantitatively determined.

According to a fourth aspect, the level of expression of the messenger RNA encoding interleukineis also determined.

The following examples are intended to further illustrate the present invention but do not limit the invention.

### EXAMPLES

### Example 1

### Search of Human ABCA5, ABCA6, ABCA9, and ABCA10 Genes in Genomic Database

Expressed sequence tags (EST) of ABCA1-like genes as described by Allikmets et al. (Hum Mol Genet, 1996, 5, 1649-1655) were used to search Genbank and UniGene nucleotide sequence databases using BLAST2 (Altschul et al, 1997, Nucleic Acids Res., 25:3389-3402). The main protein sequences databases screened were Swissprot, TrEMBL, Genpept, and PIR.

The genomic DNA analysis was performed by combination of several gene-finding programs such as GENSCAN (Burge and Karlin, 1997, J Mol Biol.; 268(1):78-94), FGENEH/FEXH (Solovyev and Salamov, 1997, lsmb; 5:294-302), and XPOUND (Thomas and Skolnick, 1994, J Math Appl Med Biol.;1 1(1):1-16). The combination of different tools lead to increase sensitivity and specificity. The second step in the genomic DNA analysis is the homology searching in the EST and protein databases. Combination of software performing database searching and software for exon/intron prediction give the best sensitive and specific results. Sequence assembly and analysis were performed using the Genetics Computer Group (GCG) sequence analysis software package.

Multiple alignments were generated by GAP software from GCG package and the Dialign2 program (Morgenstern et al, 1996, Proc Natl Acad Sci U S A.; 93(22):12098-103), the FASTA3 package (Pearson and Lipman, 1988, Proc Natl Acad Sci U S A.; 85(8):2444-8) and SIM4 (Florea et al, 1998, Genome Res. 1998 Sep. 8, 1998;(9):967-74). The specific ABCA motifs used in our process were the TMN, TMC, NBDI and NBD2 described in the literature (Broccardo et al, 1999). This corresponds in ABCA1 to residues 630-846 for the N terminal (TMN=exon 14-16) and from 1647-1877 for the C terminal set of membrane spanners (TMC=exon 36-40). The NBD corresponds to the extended nucleotide binding domain, i.e., in ABCA1 it spans from amino acids 885-1152 for the N-terminal one (NBDI=exon 18-22) and 1918-2132 for the C-terminal one (NBD2 =exon 42-47).

Sequence comparison between candidate ABCA ESTs with two overlapping BAC clones containing the microsatellite marker D17S940 (GenBank accession #AC005495, AC005922, revealed surprisingly that all these ESTs are located within this 325,000 bp. An electronic intron/exon prediction was performed by using the AC005495 and AC005922 BAC sequences, and provided transcript sequences which were predictied to correspond to the full coding sequence (CDS) of ABCA6 and ABCA9. ABCA5 gene sequences were found to be partially contain in the contig of BACs as 3′ and 5′ ends, respectively. Moreover, the analysis of the sequence revealed the ABCA10 gene.

Additional sequence information for ABCA5 was obtained by using the working draft BACs that overlap with the above described BAC contig (AC005495 and AC005922) on both 3′ and 5′ ends. A supplemental BAC working draft, I., GenBank Accession number AC007763, was then identified on the 5′ end. A parallel database mining approach based on the specific motifs search in the different Genbank subdivisions and UniGene Homo sapiens led to identification of two of these sequences (one contains a TMC motif, one contains a NBDL domain) which matched with two fragments of the BAC #AC007763.

Using exon-intron sequence of these genes, we compared the sequence of the cDNAs with the genomic sequence of the BACs (AC005495, AC005922 and AC007763) and established the approximate genomic size and respective orientation of the ABCA5, ABCA6, ABCA9, and ABCA10 genes.

### Example 2

### 5′ Extension of the Human ABCA5, ABCA6, ABCA9, and ABCA10 cDNA

This Example describes the isolation and identification of cDNA molecules encoding the full length human ABCA5, ABCA6, ABCA9, and ABCA10 protein. 5′ extension of the partial ABCA5, ABCA6, ABCA9, and ABCA10 cDNA sequence was performed by using a combination of 5′ RACE and RT-PCR on liver, heart, or testis.

Oligonucleotide primers allowing to distinguish novel ABCA5-6 and 9-10 genes from other family members, were designed taking advantage of the exonic/intronic prediction of the genomic sequence and used to identify specific cDNA transcript by RT-PCR on RNA from various human tissues. With the exception of ABCA10 that required an additional cloning step, all RT-PCR products were directly sequenced. In the case of ABCA6, ABCA9, and ABCA10 a 5′ RACE step was also performed in order to confirm the initiator ATG codon. The identification of the full CDS of ABCA5 was obtained by linking the 3 potential fragments of the transcript by RT-PCR and direct sequencing. Finally, full ORF sequences of these new genes that belong to the same chromosome 17 cluster were determined.

Reverse Transcription

In a total volume of 11.5 μl, 500 ng of mRNA poly(A)+(Clontech) mixed with 500 ng of oligodt are denaturated at 70° C. for 10 min and then chilled on ice. After addition of 10 units of RNAsin, 10 mM DTT, 0.5 mM dNTP, Superscript first strand buffer and 200 units of Superscript II (Life Technologies), the reaction is incubated for 45 min at 42° C. We used poly(A) mRNA from liver, heart, brain and lung for ABCA9, from testis for ABCA5, from testis and heart for ABCA10.

PCR

Each polymerase chain reaction contained 400 μM each dNTP, 2 units of Thermus aquaticus (Taq) DNA polymerase (Ampli Taq Gold; Perkin Elmer), 0.5 μM each primer, 2.5 mM MgCl₂, PCR buffer and 50 ng of DNA, or about 25 ng of cDNA, or 1/50e of primary PCR mixture. Reactions were carried out for 30 cycles in a Perkin Elmer 9700 thermal cycler in 96-well microtiter plates. After an initial denaturation at 94° C. for 10 min, each cycle consisted of: a denaturation step of 30 s (94° C.), a hybridization step of 30 s (64° C. for 2 cycles, 61° C. for 2 cycles, 58° C. for cycles and 55° C. for 28 cycles), and an elongation step of 1 min/kb (72° C.). PCR ended with a final 72° C. extension of 7 min. In case of RT-PCR, control reactions without reverse transcriptase and reactions containing water instead of cDNA were performed for every sample.

DNA Sequencing

PCR products are analyzed and quantified by agarose gel electrophoresis, purified with a P100 column. Purified PCR products were sequenced using ABI Prism BigDye terminator cycle sequencing kit (Perkin Elmer Applied Biosystems). The sequence reaction mixture was purified using Microcon-100 microconcentrators (Amicon, Inc., Beverly). Sequencing reactions were resolved on an ABI 377 DNA sequencer (Perkin Elmer Applied Biosystems) according to manufacturer's protocol (Applied Biosystems, Perkin Elmer).

5′ Rapid Amplification of cDNA Ends (RACE)

5′ RACE analysis was performed using the SMART RACE cDNA amplification kit (Clontech, Palo Alto, Calif.). Human testis, liver and heart polyA+RNA (Clontech) were used as template to generate the 5′ SMART cDNA library according to the manufacturer's instructions. First-amplification primers and nested primers were designed from the cDNA sequence. Amplimers of the nested PCR were cloned. Insert of specific clones are amplified by PCR with universal primers (Rev and −21) and sequenced on both strands. Primers ABC-A6_L1, L2, ABC-A9_L1, L2, and ABC-A10_L1, L2 were used to identify 5′ ends of ABC-A6, ABC-A9, and ABC-A10 respectively.

Primers

Oligonucleotides were selected using Prime from GCG package or Oligo 4 (National Biosciences, Inc.) softwares. Primers were ordered from Life Technologies, Ltd and used without further purification (Table 15).

TABLE 15
RT-PCR and 5′RACE primers

| SEQ | | | |
|---|---|---|---|
| ID NO: | Name | Sequence | Orientation |
| 127 | ABCA5_A | CAGTGACTATGTATCCGTG | Forward |
| 128 | ABCA5_B | GATGGTTTCTCCTCACAAC | Reverse |
| 129 | ABCA5_C | CACCAGACAATGAGGATGA | Forward |
| 130 | ABCA5_D | GCTATATTCTTCAATGGCA | Reverse |
| 131 | ABCA5_E | CCTAGAAGTAGACCGCCTT | Forward |
| 132 | ABCA5_G | GTTGTGAGGAGAAACCATC | Forward |
| 133 | ABCA5_H | CTGGATGGTTTCAGTCACA | Reverse |
| 134 | 345770_A | CAGAAAAGCCAATCGGGTG | Forward |
| 135 | 345770_B | CCAGGTATATGTTGTTTAACCAG | Reverse |
| 136 | 345770_C | GGGTCAGATTACTGCCTTAC | Forward |
| 137 | 345770_D | GAACATTGAAGAACCAACAC | Reverse |
| 138 | 345770_F | GTAAGGCAGTAATCTGACCC | Reverse |
| 139 | 445188_B | GGAAACTGGACAGAATGC | Reverse |
| 140 | 445188_C | CTACCCTATTTCACATGCC | Forward |
| 141 | 445188_D | GTTTCTCCCATAATAACAGC | Reverse |
| 142 | 445188_E | GCTGTTATTATGGGAGAAAC | Forward |
| 143 | 445188_L1 | AGACTACAGTAACAAAAGCCTAGTGCAGCC | Reverse |
| 144 | 445188_L2 | ATCCAATCCTATTAGTGTGACAAAGGCTTG | Reverse |
| 145 | ABCA6_A | TCAGCAAACCAAAGCACTTC | Forward |
| 146 | ABCA6_B | TGACATCAACTCCTCCATCAC | Reverse |
| 149 | ABCA6_C | CCCTGTGATGGAGGAGTTG | Forward |
| 148 | ABCA6_C1 | TCATTGCTGGGATGGATATG | Forward |
| 154 | ABCA6_D | AAGGGTCAGGAAAAATTACACC | Reverse |
| 151 | ABCA6_D1 | GAATGCTGAATCTTGGAGAC | Reverse |
| 153 | ABCA6_E | TGGTGTAATTTTTCCTGACCC | Forward |
| 152 | ABCA6_E1 | GATTCAGATTATCAAACTGG | Forward |
| 157 | ABCA6_F | CCACTTCCTTTAGATGAATCCC | Reverse |
| 155 | ABCA6_F1 | GGAATTCAGGAGCTACTGG | Reverse |
| 158 | ABCA6_G | AAGTGGAACAAGAGGTACAACG | Forward |
| 156 | ABCA6_G1 | GATTGTCTGTTCCAACAGAAGG | Forward |
| 160 | ABCA6_H | GGGGATGTGATGAGTAATGAAG | Reverse |
| 159 | ABCA6_H1 | ATGGTAATCCCAAAAGTCAGC | Reverse |
| 161 | ABCA6_I | CTTCATTACTCATCACATCCCC | Forward |
| 163 | ABCA6_J | GATCAACAGGCTGGTACGG | Reverse |
| 162 | ABCA6_K | ACAACTTCCCCAGGAACCC | Forward |
| 165 | ABCA6_L | TGCCCACACCAGTAAGCAG | Reverse |
| 164 | ABCA6_M | CAAGAAAAATGCTAAGTCCCAG | Forward |
| 166 | ABCA6_N | GAAAATCAGTGGCACTCAATTC | Reverse |
| 167 | ABCA6_O | TGCCACTGATTTTCTAGTCTGC | Forward |
| 169 | ABCA6_P | CCTTTCAGTTCCACCTCTCC | Reverse |
| 168 | ABCA6_Q | CTGGGATCACAAAGCCAAC | Forward |
| 171 | ABCA6_R | AATACCTTTCCTGCCCTGC | Reverse |
| 170 | ABCA6_S | TCCACACTGAGATTCTGAAGC | Forward |
| 172 | ABCA6_T | GCCTGACTCTTTGGGTGAC | Reverse |
| 147 | ABCA6_L1 | GTACATGAAAACTCACCATATCCATCCC | Reverse |
| 146 | ABCA6_L2 | GCAAGTGCTGTTTTATTCATTATCTGCTG | Reverse |
| 173 | ABCA9_A | TGAGCGTGGGTCAGCAAAC | Forward |
| 174 | ABCA9_B | GCAACTCCTCCTTGGGCAAC | Reverse |
| 175 | ABCA9_C | TTTGTTGCCCAAGGAGGAG | Forward |
| 176 | ABCA9_D | GGAAAAACAAGGGAGAACATCG | Reverse |
| 177 | ABCA9_E1 | GCCCACTTGGATTCTTCAC | Forward |
| 178 | ABCA9_F1 | CCACACCTTTCAAAGCTTCTAC | Reverse |
| 179 | ABCA9_G1 | ATGTGGTCCTTGAGAATGAAAC | Forward |
| 180 | ABCA9_G2 | ACTGTGAAAGAAAACCTCAGGC | Forward |
| 181 | ABCA9_H1 | CTTCATGTGGCAAAATCCC | Reverse |
| 182 | ABCA9_H2 | TGTGCTGTCAATTTGGCATC | Reverse |
| 183 | ABCA9_I | AAGAAGAAATGGGGCATAGG | Forward |
| 184 | ABCA9_J | TGTATTTGGAGACAGTTCCCAC | Reverse |
| 185 | ABCA9_K1 | AACAATCAGTGGCGTGGCG | Forward |
| 202 | ABCA9_L1(RACE) | CTTGGGTAGTTTTGGATTCAGGTGC | Reverse |
| 186 | ABCA9_L1(CV) | GACATCCAGGAGGACAGGAAAG | Reverse |
| 203 | ABCA9_L2 | AGATCCATTGAAGACATTTGAGGAGTG | Reverse |
| 187 | ABCA9_M | GCAGCCTCTTTCACTCCATAC | Forward |
| 188 | ABCA9_M1 | CATTGTGTCAGGTGATGAAAAG | Forward |
| 189 | ABCA9_N | TTCATTTCTAGGCATCGCAG | Reverse |
| 190 | ABCA9_N1 | CATTAGCAGGAGGATCAAAAAG | Reverse |
| 191 | ABCA9_O | TCTAGGGCTATTTTTTGGCAC | Forward |
| 192 | ABCA9_P | CGCTCCCTTTCAAAATCAC | Reverse |
| 193 | ABCA9_Q1 | TGCGAGACTTTGATGAGACAC | Forward |
| 194 | ABCA9_T2 | AGACCATCAGGGAGGAGAAC | Reverse |
| 195 | ABCA9_U | TGTGCCAGCAACCAAATC | Forward |
| 196 | ABCA9_U1 | GCTGGAGATGAAGCTGAAGAAC | Forward |
| 201 | ABCA9_U2 | AAGCATGATGTAGTAGTGACCC | Forward |
| 197 | ABCA9_V1 | TTTCCACTTCACCGAGGG | Reverse |
| 198 | ABCA9_W | CCATGTTTTGTCTGTTGTGCC | Forward |
| 199 | ABCA9_X | CACCCATCAACCCATCATCTAC | Reverse |
| 200 | ABCA9_Z | AGGCACAACAGACAAAACATGG | Reverse |
| 204 | ABCA10_A | GATTGACATACATTTGCTTC | Forward |
| 205 | ABCA10_B | TACAGTGAAGAGAAATCCAG | Reverse |
| 206 | ABCA10_C | TGGAATTAGACATGCAAA | Forward |
| 207 | ABCA10_D | TGAAGAGGATAAGTCGGTC | Reverse |
| 208 | ABCA10_E | TATAATCGCTGATGCTGC | Reverse |
| 212 | ABCA10_I | AGATAAGCGTGCGTCAAC | Forward |
| 215 | ABCA10_N | TCATCAACATTTCCCAGC | Reverse |
| 216 | ABCA10_AA | GAAATACTGGAGATGAGTCTG | Forward |
| 217 | ABCA10_AB | GAGCTTAAGAGCTTCCACC | Reverse |
| 213 | ABCA11_C | TCTTATGGGAATTGTTAGCA | Forward |
| 214 | ABCA11_H | TTATGACTGGTTCCTCCTC | Reverse |
| 209 | ABCA10_L1 | ACCAGGCCAGAGTCATTAAACTGATC | Reverse |
| 210 | ABCA10_L2 | CCGAAAAGATGCACAAATATAGCCC | Reverse |
| 211 | ABCA10_U2 | CTCAAAACTTCATTCTAATTGTGCCC | Forward |

### Example 3

### Tissue Distribution of the Transcripts of the ABCA5, ABCA6, ABCA9, and ABCA10 Genes According to the invention.

The profile of expression of the polynucleotides according to the present invention is determined according to the protocols for PCR-coupled reverse transcription and Northern blot analysis described in particular by Sambrook et al. (1989, Molecular cloning: a laboratory manual. 2ed. Cold Spring Harbor Laboratory, Cold spring Harbor, N.Y.).

For example, in the case of an analysis by reverse transcription, a pair of primers as described above may be synthesized from a cDNA of the human ABCA5, ABCA6, ABCA9, and ABCA10 genes. This primer pair may be used to detect the corresponding ABCA5, ABCA6, ABCA9, and ABCA10 cDNAs.

The polymerase chain reaction (PCR) is carried out on cDNA templates corresponding to retrotranscribed polyA⁺mRNAs (Clontech). The reverse transcription to cDNA is carried out with the enzyme SUPERSCRIPT II (GibcoBRL, Life Technologies) according to the conditions described by the manufacturer. The polymerase chain reaction is carried out according to standard conditions, in 20 μl of reaction mixture with 25 ng of cDNA preparation. The reaction mixture is composed of 400 μM of each of the dNTPs, 2 units of Thermus aquaticus (Taq) DNA polymerase (Ampli Taq Gold; Perkin Elmer), 0.5 μM of each primer, 2.5 mM MgCl2, and PCR buffer. Thirty four PCR cycles [denaturing 30 seconds at 94° C., annealing of 30 seconds divided up as follows during the 34 cycles: 64° C. (2 cycles), 61° C. (2 cycles), 58° C. (2 cycles), and 55° C. (28 cycles), and an extension of one minute per kilobase at 72° C.] are carried out after a first step of denaturing at 94° C. for 10 minutes using a Perkin Elmer 9700 thermocycler. The PCR reactions are visualized on agarose gel by electrophoresis. The cDNA fragments obtained may be used as probes for a Northern blot analysis and may also be used for the exact determination of the nucleotide sequence.

Northern Blot Analysis

To study mRNA expression of the ABCA5, ABCA6, ABCA9, and ABCA10 genes, human MTN (Multiple Tissue Northern) blots (Human II 7759-1, Human 7760-1, and human fetal II 7756-1, Clontech) were hybridized with specific pools of probes consisting in two amplimers: ABCA5_A-ABCA5_B and 445188_C-445188_D for ABC-A5, ABCA6_A-ABCA6_B and ABCA6_S-ABCA6_T for ABC-A6, ABCA9_A-ABCA9-B and ABCA9_M1-ABCA9_N1 for ABC-A9. A unique RT-PCR product obtained between ABCA10_I-ABCA10_B was used for ABC-A10 (Table 15).

Preparation of the Probe

PCR products were gel-purified using Qiaquick® column (Qiagen). 10-20 ng of purified PCR product were radiolabelled with [α³²P]dCTP (Amersham; 6000 Ci/mmol, 10 mCi/ml) by the random priming method (Rediprime kit; Amersham) according to the manufacturer's protocol. Unincorporated radioactive nucleotides were separated from the labelled probe by filtration on a G50 microcolumn (Pharmacia). Probe was competed with 50 μg of denatured human Cot1 DNA during 2 hours at 65° C.

Hybridization

Prehybridization of Northern blot (6 hours at 42° C.) with hybridization solution (5×SSPE, 5×Denhardt's, 2.5% Dextran, 0.5% SDS, 50% formamide, 100 μg/ml denaturated salmon sperm DNA, 40 μg denatured human DNA) was followed by hybridization with radiolabelled probe (2.10⁶ cpm/ml hybridization solution) and 40 pg of denatured human DNA. Filters were washed in 2×SSC for 30 min at room temperature, twice in 2×SSC-0.1% SDS for 10 min at 65° C. and twice in 1×SSC-0.1% SDS for 10 min at 65° C. Northern blot were analyzed after overnight exposure on the Storm (Molecular Dynamics, Sunnyvale, Calif.). The human transferrin probe was used to control the amount of RNA in each lane of the membrane.

### Example 4

### Construction of the Expression Vector Containing the Complete cDNA of ABCA5, ABCA6, ABCA9, or ABCA10 in Mammalian Cells

The ABCA5, ABCA6, ABCA9, or ABCA10 genes may be expressed in mammalian cells. A typical eukaryotic expression vector contains a promoter which allows the initiation of the transcription of the mRNA, a sequence encoding the protein, and the signals required for the termination of the transcription and for the polyadenylation of the transcript. It also contains additional signals such as enhancers, the Kozak sequence and sequences necessary for the splicing of the mRNA. An effective transcription is obtained with the early and late elements of the SV40 virus promoters, the retroviral LTRs or the CMV virus early promoter. However, cellular elements such as the actin promoter may also be used. Many expression vectors may be used to carry out the present invention, an example of such a vector is pcDNA3 (Invitrogen).

### Example 5

### Production of Normal and Mutated ABCA5, ABCA6, ABCA9, or ABCA10 Polypeptides

The normal ABCA5, ABCA6, ABCA9, or ABCA10 polypeptides encoded by complete corresponding cDNAs whose isolation is described in Example 2, or the mutated ABCA5, ABCA6, ABCA9, or ABCA10 polypeptides whose complete cDNA may also be obtained according to the techniques described in Example 2, may be easily produced in a bacterial or insect cell expression system using the baculovirus vectors or in mammalian cells with or without the vaccinia virus vectors. All the methods are now widely described and are known to persons skilled in the art. A detailed description thereof will be found for example in F. Ausubel et al. (1989, Current Protocols in Molecular Biology, Green Publishing Associates and Wiley Interscience, N.Y).

### Example 6

### Production of an Antibody Directed Against One of the Mutated ABCA5, ABCA6, ABCA9, or ABCA10 Polypeptides

The antibodies in the present invention may be prepared by various methods (Current Protocols In Molecular Biology Volume 1 edited by Frederick M. Ausubel, Roger Brent, Robert E. Kingston, David D. Moore, J. G. Seidman, John A. Smith, Kevin Struhl—Massachusetts General Hospital Harvard Medical School, chapter 11, 1989). For example, the cells expressing a polypeptide of the present invention are injected into an animal in order to induce the production of serum containing the antibodies. In one of the methods described, the proteins are prepared and purified so as to avoid contaminations. Such a preparation is then introduced into the animal with the aim of producing polyclonal antisera having a higher activity.

In the preferred method, the antibodies of the present invention are monoclonal antibodies. Such monoclonal antibodies may be prepared using the hybridoma technique (Köhler et al, 1975, Nature, 256:495; Köhler et al, 1976, Eur. J. Immunol. 6:292; Köhler et al, 1976, Eur. J. Immunol., 6:511; Hammeling et al., 1981, Monoclonal Antibodies and T-Cell Hybridomas, Elsevier, N.Y., pp. 563-681). In general, such methods involve immunizing the animal (preferably a mouse) with a polypeptide or better still with a cell expressing the polypeptide. These cells may be cultured in a suitable tissue culture medium. However, it is preferable to culture the cells in an Eagle medium (modified Earle) supplemented with 10% fetal bovine serum (inactivated at 56° C.) and supplemented with about 10 g/l of nonessential amino acids, 1000 U/mI of penicillin and about 100 μg/ml of streptomycin.

The splenocytes of these mice are extracted and fused with a suitable myeloma cell line. However, it is preferable to use the parental myeloma cell line (SP20) available from the ATCC. After fusion, the resulting hybridoma cells are selectively maintained in HAT medium and then cloned by limiting dilution as described by Wands et al. (1981, Gastroenterology, 80:225-232). The hybridoma cells obtained after such a selection are tested in order to identify the clones secreting antibodies capable of binding to the polypeptide.

Moreover, other antibodies capable of binding to the polypeptide may be produced according to a 2-stage procedure using anti-idiotype antibodies such a method is based on the fact that the antibodies are themselves antigens and consequently it is possible to obtain an antibody recognizing another antibody. According to this method, the antibodies specific for the protein are used to immunize an animal, preferably a mouse. The splenocytes of this animal are then used to produce hybridoma cells, and the latter are screened in order to identify the clones which produce an antibody whose capacity to bind to the specific antibody-protein complex may be blocked by the polypeptide. These antibodies may be used to immunize an animal in order to induce the formation of antibodies specific for the protein in a large quantity.

It is preferable to use Fab and F(ab′)2 and the other fragments of the antibodies of the present invention according to the methods described here. Such fragments are typically produced by proteolytic cleavage with the aid of enzymes such as Papafn (in order to produce the Fab fragments) or Pepsin (in order to produce the F(ab′)2 fragments). Otherwise, the secreted fragments recognizing the protein may be produced by applying the recombinant DNA or synthetic chemistry technology.

For the in vivo use of antibodies in humans, it would be preferable to use “humanized” chimeric monoclonal antibodies. Such antibodies may be produced using genetic constructs derived from hybridoma cells producing the monoclonal antibodies described above. The methods for producing the chimeric antibodies are known to persons skilled in the art (for a review, see : Morrison (1985. Science 229:1202); Oi et al., (1986, Biotechnique, 4:214); Cabilly et al., U.S. Pat. No. 4,816,567 ; Taniguchi et al., EP 171496; Morrison et al., EP 173494; Neuberger et al., WO 8601533; Robinson et al., WO 8702671; Boulianne et al; (1984, Nature, 312:643); and Neuberger et al., (1985, Nature, 314:268).

### Example 7

### Determination of Polymorphisms/mutations in the ABCA5, ABCA6, ABCA9, or ABCA10 Genes

The detection of polymorphisms or of mutations in the sequences of the transcripts or in the genomic sequence of the ABCA5, ABCA6, ABCA9, or ABCA10 genes may be carried out according to various protocols. The preferred method is direct sequencing.

For patients from whom it is possible to obtain an mRNA preparation, the preferred method consists in preparing the cDNAs and sequencing them directly. For patients for whom only DNA is available, and in the case of a transcript where the structure of the corresponding gene is unknown or partially known, it is necessary to precisely determine its intron-exon structure as well as the genomic sequence of the corresponding gene. This therefore involves, in a first instance, isolating the genomic DNA BAC or cosmid clone(s) corresponding to the transcript studied, sequencing the insert of the corresponding clone(s) and detemrining the intron-exon structure by comparing the cDNA sequence to that of the genomic DNA obtained.

The technique of detection of mutations by direct sequencing consists in comparing the genomic sequences of the ABCA5, ABCA6, ABCA9, or ABCA10 gene obtained from homozygotes for the disease or from at least 8 individuals (4 individuals affected by the pathology studied and 4 individuals not affected) or from at least 32 unrelated individuals from the studied population. The sequence divergences constitute polymorphisms. All those modifying the amino acid sequence of the wild-type protein may be mutations capable of affecting the function of said protein which it is preferred to consider more particularly for the study of cosegregation of the mutation and of the disease (denoted genotype-phenotype correlation) in the pedigree, or of a pharmacological response to a therapeutic molecule in the pharmacogenomic studies, or in the studies of caselcontrol association for the analysis of the sporadic cases.

### Example 8

### Identification of a Causal Gene for a Disease Linked to a Deficiency in the Transport of Cholesterol and Inflammatory Lipid Substances by Causal Mutation or a Transcriptional Difference

Among the mutations identified according to the method described in Example 7, all those associated with the disease phenotype are capable of being causal. Validation of these results is made by sequencing the gene in all the affected individuals and their relations (whose DNA is available).

Moreover, Northern blot or RT-PCR analysis, according to the methods described in Example 2, using RNA specific to affected or nonaffected individuals makes it possible to detect notable variations in the level of expression of the gene studied, in particular in the absence of transcription of the gene.

### Example 9

### Construction of Recombinant Vectors Comprising a Nucleic Acid Encoding Any One of ABCA5, ABCA6, ABCA9, and ABCA10 Proteins

Synthesis of a Nucleic Acid Encoding Any One of Human ABCA5, ABCA6. ABCA9, or ABCA10 Proteins

Total RNA (500 ng) isolated from a human cell (for example, placental tissue, Clontech, Palo Alto, Calif., USA, or THP1 cells) may be used as source for the synthesis of the cDNA of the human ABCA5, ABCA6, ABCA9, and ABCA10 genes. Methods to reverse transcribe mRNA to cDNA are well known in the art. For example, one may use the system “Superscript one step RT-PCR (Life Technologies, Gaithersburg, Md., USA).

Oligonucleotide primers specific for ABCA5, ABCA6, ABCA9, and ABCA10 cDNAs may be used for this purpose, containing sequences as set forth in any of SEQ ID NOS: 127-217. These oligonucleotide primers may be synthesized by the phosphoramidite method on a DNA synthesizer of the ABI 394 type (Applied Biosystems, Foster City, Calif., USA).

Sites recognized by the restriction enzyme NotI may be incorporated into the amplified ABCA5, ABCA6, ABCA9, and ABCA10 cDNAs to flank the cDNA region desired for insertion into the recombinant vector by a second amplification step using 50 ng of human ABCA5, ABCA6, ABCA9, and ABCA10 cDNAs as template, and 0.25 μM of the ABCA5, ABCA6, ABCA9, and ABCA10 specific oligonucleotide primers used above containing, at their 5′ end, the site recognized by the restriction enzyme NotI (5′-GCGGCCGC-3′), in the presence of 200 μM of each of said dideoxynucleotides dATP, dCTP, dTTP and dGTP as well as the Pyrococcus furiosus DNA polymerase (Stratagene, Inc. La Jolla, Calif., USA).

The PCR reaction may be carried out over 30 cycles each comprising a step of denaturation at 95° C. for one minute, a step of renaturation at 50° C. for one minute and a step of extension at 72° C. for two minutes, in a thermocycler apparatus for PCR (Cetus Perkin Elmer Norwalk, Conn., USA).

Cloning of the cDNA of the Human ABCA5, ABCA6, ABCA9, and ABCA10 Genes Into an Expression Vector

The human ABCA5, ABCA6, ABCA9, and ABCA10 cDNA inserts may then be cloned into the NotI restriction site of an expression vector, for example, the pCMV vector containing a cytomegalovirus (CMV) early promoter and an enhancer sequence as well as the SV40 polyadenylation signal (Beg et al., 1990, PNAS, 87:3473; Applebaum-Boden, 1996, JCI 97), in order to produce an expression vector designated pABCA5, pABCA6, pABCA9, and pABCA10.

The sequence of the cloned cDNA can be confirmed by sequencing on the two strands using the reaction set “ABI Prism Big Dye Terminator Cycle Sequencing ready” (marketed by Applied Biosystems, Foster City, Calif., USA) in a capillary sequencer of the ABI 310 type (Applied Biosystems, Foster City, Calif., USA).

Construction of a Recombinant Adenoviral Vector Containing the cDNA of the Human ABCA5, ABCA6, ABCA9, and ABCA10 Genes

Modification of the expression vector pCMV-β:

The β-galactosidase cDNA of the expression vector pCMV-β (Clontech, Palo Alto, Calif., USA, Gene Bank Accession No. UO2451) may be deleted by digestion with the restriction endonuclease NotI and replaced with a multiple cloning site containing, from the 5′ end to the 3′ end, the following sites: NotI, AscI, RsrII, AvrII, SwaI, and NotI, cloned at the region of the NotI restriction site. The sequence of this multiple cloning site is:

5′-CGGCCGCGGCGCGCCCGGACCGCCTAGGATTTAAATCGCGGCCCGCG-3′.

The DNA fragment between the EcoRI and SanI sites of the modified expression vector pCMV may be isolated and cloned into the modified Xbal site of the shuttle vector PXCXII (McKinnon et al., 1982, Gene, 19:33; McGrory et al., 1988, Virology, 163:614).

Modification of the shuttle vector pXCXII:

A multiple cloning site comprising, from the 5′ end to the 3 end the XbaI, EcoRI, SfiI, PmeI, NheI, SrfI, PacI, SalI and XbaI restriction sites having the sequence:

5′CTCTAGAATTCGGCCTCCGTGGCCGTTTAAACGCTAGCGCCCGGGCTTAATTAAGTCGACTCTAGAGC-3′, may be inserted at the level of the XbaI site (nucleotide at position 3329) of the vector pXCXII (McKinnon et al., 1982, Gene 19:33; McGrory et al., 1988, Virology, 163:614).

The EcoRI-SalI DNA fragment isolated from the modified vector pCMV-β containing the CMV promoter/enhancer, the donor and acceptor splicing sites of FV40 and the polyadenylation signal of FV40 may then be cloned into the EcoRI-SalI site of the modified shuttle vector pXCX, designated pCMV-11.

Preparation of the Shuttle Vector pAD12-ABCA

The human ABCA5, ABCA6, ABCA9, and ABCA10 cDNAs are obtained by an RT-PCR reaction, as described above, and cloned at the level of the NotI site into the vector pCMV-12, resulting in the obtaining of the vector pCMV-ABCA5, pCMV-ABCA6, pCMV-ABCA9, and pCMV-ABCA10.

Construction of the ABC1 Recombinant Adenovirus

The recombinant adenovirus containing the human ABCA5, ABCA6, ABCA9, and ABCA10 cDNAs may be constructed according to the technique described by McGrory et al. (1988, Virology, 163:614).

Briefly, the vector pAD12-ABCA is cotransfected with the vector tGM17 according to the technique of Chen and Okayama (1987, Mol Cell Biol., 7:2745-2752).

Likewise, the vector pAD12-Luciferase was constructed and cotransfected with the vector pJM17.

The recombinant adenoviruses are identified by PCR amplification and subjected to two purification cycles before a large-scale amplification in the human embryonic kidney cell line HEK 293 (American Type Culture Collection, Rockville, Md., USA).

The infected cells are collected 48 to 72 hours after their infection with the adenoviral vectors and subjected to five freeze-thaw lysing cycles.

The crude lysates are extracted with the aid of Freon (Halocarbone 113, Matheson Product, Scaucus, N.J. USA), sedimented twice in cesium chloride supplemented with 0.2% murine albumine (Sigma Chemical Co., St Louis, Mo., USA) and dialysed extensively against buffer composed of 150 nM NaCl, 10 mM Hepes (pH 7,4), 5 mM KCl, 1 mM MgCl₂, and 1 mM CaCl₂.

The recombinant adenoviruses are stored at −70° C. and titrated before their administration to animals or their incubation with cells in culture.

The absence of wild-type contaminating adenovirus is confirmed by screening with the aid of PCR amplification using oligonucleotide primers located in the structural portion of the deleted region.

Validation of the Expression of the Human ABCA5, ABCA6, ABCA9, and ABCA10 cDNAs

Polyclonal antibodies specific for a human ABCA5, ABCA6, ABCA9, and ABCA10 polypeptides may be prepared as described above in rabbits and chicks by injecting a synthetic polypeptide fragment derived from an ABC1 protein, comprising all or part of an amino acid sequence as described in SEQ ID NOS:5-8. These polyclonal antibodies are used to detect and/or quantify the expression of the human ABCA5, ABCA6, ABCA9, and ABCA10 genes in cells and animal models by immunoblotting and/or immunodetection.

The biological activity of ABCA5-6, 9-10 may be monitored by quantifying the cholesterol fluxes induced by apoA-I using cells transfected with the vector pCMV-ABCI which have been loaded with cholesterol (Remaley et al., 1997, ATVB, 17:1813).

Expression in Vitro of the Human ABCA5, ABCA6, ABCA9, and ABCA10 cDNAs in Cells

Cells of the HEK293 line and of the COS-7 line (American Tissue Culture Collection, Bethesda, Md., USA), as well as fibroblasts in primary culture derived from Tangier patients or from patients suffering from hypo-alphalipoproteinemia are transfected with the expression vector pCMV-ABCA5, pCMV-ABCA6, pCMV-ABCA9, and pCMV-ABCA10 (5-25 μg) using Lipofectamine (BRL, Gaithersburg, Md., USA) or by coprecipitation with the aid of calcium chloride (Chen et al., 1987, Mol Cell Biol., 7:2745-2752).

These cells may also be infected with the vector pABCA5-AdV, pABCA6-AdV, pABCA9-AdV, and pABCA10-AdV (Index of infection, MOI=10).

The expression of human ABCA5-6, 9-10 may be monitored by immunoblotting as well as by quantification of the efflux of cholesterol induced by apoA-1 using transfected and/or infected cells.

Expression in Vivo of the ABCA5, ABCA6, ABCA9, and ABCA10 Genes in Various Animal Models

An appropriate volume (100 to 300 μl) of a medium containing the purified recombinant adenovirus (pABCA-AdV or pLucif-AdV) containing from 10⁸ to 10⁹ lysis plaque-forming units (pfu) are infused into the Saphenous vein of mice (C57BL/6, both control mice and models of transgenic or knock-out mice) on day 0 of the experiment.

The evaluation of the physiological role of the ABCA5, ABCA6, ABCA9, and ABCA10 proteins in the transport of cholesterol or inflammatory lipid substances is carried out by determining the total quantity of cholesterol or appropriate inflammatory lipid substances before (day zero) and after (days 2, 4, 7, 10, 14) the administration of the adenovirus.

Kinetic studies with the aid of radioactively labelled products are carried out on day 5 after the administration of the vectors rLucif-AdV and rABCA-AdV in order to evaluate the effect of the expression of ABCA5, ABCA6, ABCA9, and ABCA10 on the transport of cholesterol and inflammatory lipid substances.

Furthermore, transgenic mice and rabbits overexpressing the ABCA5, ABCA6, ABCA9, and ABCA10 genes may be produced, in accordance with the teaching of Vaisman (1995) and Hoeg (1996) using constructs containing the human ABCA5, ABCA6, ABCA9, and ABCA10 cDNAs under the control of endogenous promoters such as ABCA5, ABCA6, ABCA9, and ABCA10, or CMV or apoE.

The evaluation of the long-term effect of the expression of ABCA5, ABCA6, ABCA9, and ABCA10 on the kinetics of the lipids involved in the mediation of the inflammation may be carried out as described above.

### Example 10

### Isotopic in Situ Hybridization Study of the ABCA9 Gene

In situ hybridization experiments were performed using a radiolabeled cRNA probe of 330 bp corresponding to nucleotides 1 to 330 of nucleotide sequence of SEQ ID NO: 3. The 330 bp insert was subcloned and transcribed in vitro with T7 (antisense) and SP6 (sense) RNA polymerases in the presence of ³⁵S-uridine 5′-triphosphate. After transcription, the probes were column-purified and separated by electrophoresis on a 5% polyacrylamide gel to confirm size and purity.

Serial artery and heart tissue sections were digested with Proteinase K and hybridized with the probes at a concentration of approximately 3.4×10⁷ dpm/ml for 18 hours at 55° C. Following hybridization, the slides were treated with RNAse A and washed stringently in 0.1×SSC at 60° C. for 2 hours. The slides were then coated with Kodak NTB-2 emulsion, exposed for 14 days at 4° C., and developed using Kodak D-19 Developer and Fixer. Slides were stained with hematoxylin and eosin (H&E) and imaged using a DVC 1310C camera coupled to a Nikon microscope.

Two control probes were used in these studies. All tissues were screened initially with a probe for beta-actin mRNA to ensure that RNAs were preserved within the archival paraffin samples. Adjacent serial sections were also hybridized with a sense control riboprobe that was derived from the same region of the gene as the antisense probe. At these hybridization and wash stringencies, the sense control probe tended to produce a background signal across all tissues that was not associated with particular cell types and that appeared to be due to nonspecific sticking of the sense probe to the tissues. The nonspecific background produced by the antisense probe was less than that observed with the sense probe, and the positive antisense signals described in the accompanying report were specifically cell-associated and higher than the background signals present in nonreactive cell types.

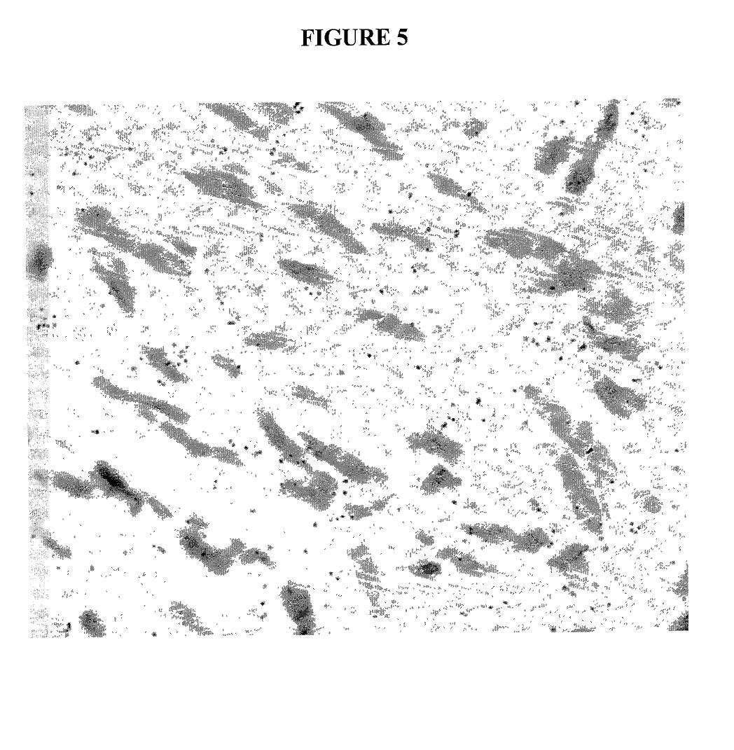

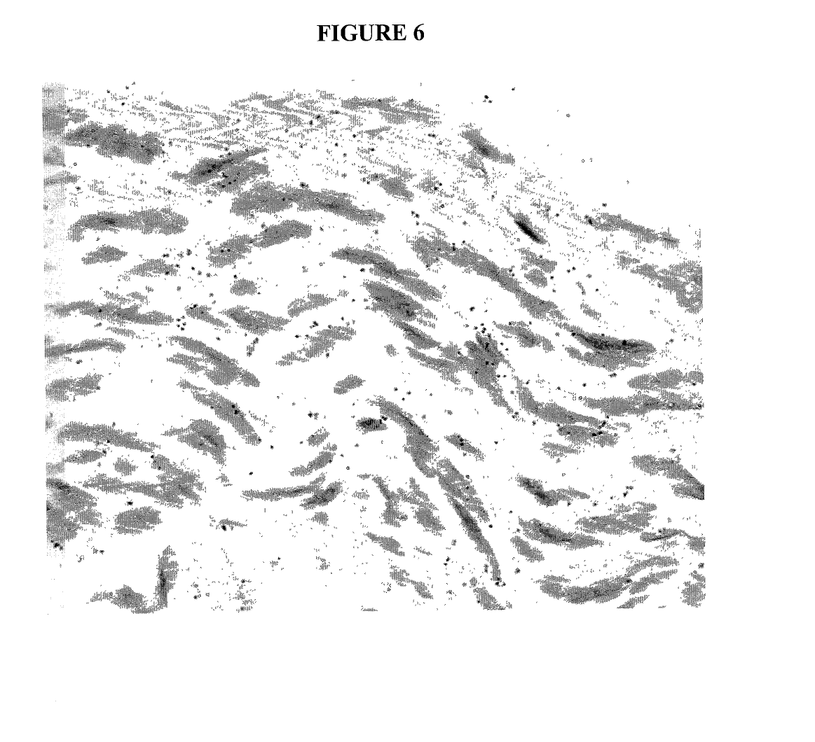

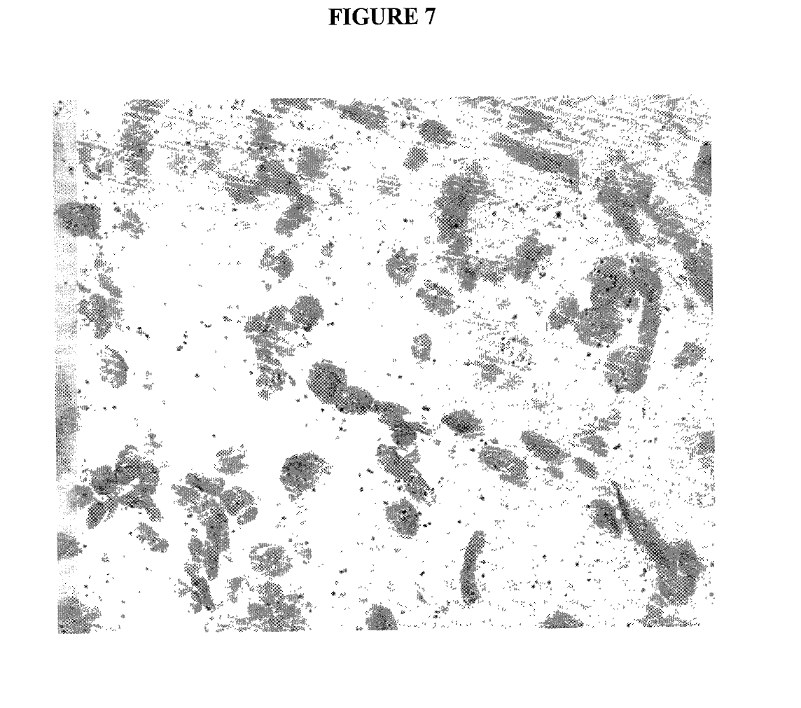

FIG. 5 was a section of normal renal artery obtained at nephrectomy from an 80-year-old female with renal cell carcinoma, and showed a faint hybridization of medial smooth muscle in both arteries and veins. Adventitial nerves showed occasional faint positivity in Schwann cells (FIG. 6). Also, in an adjacent ganglion, ganglion cells and Schwann cells were both faintly positive (FIG. 7).

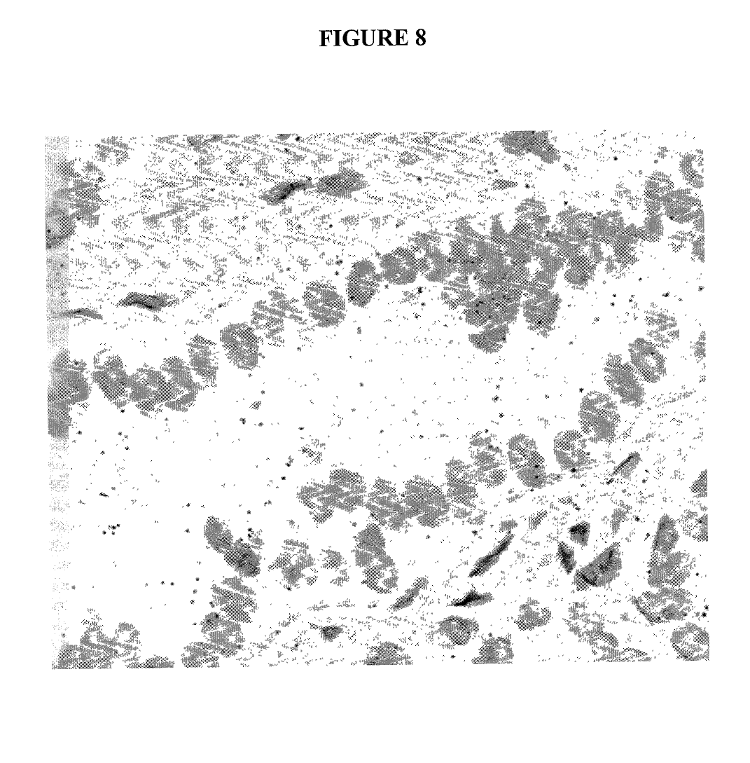

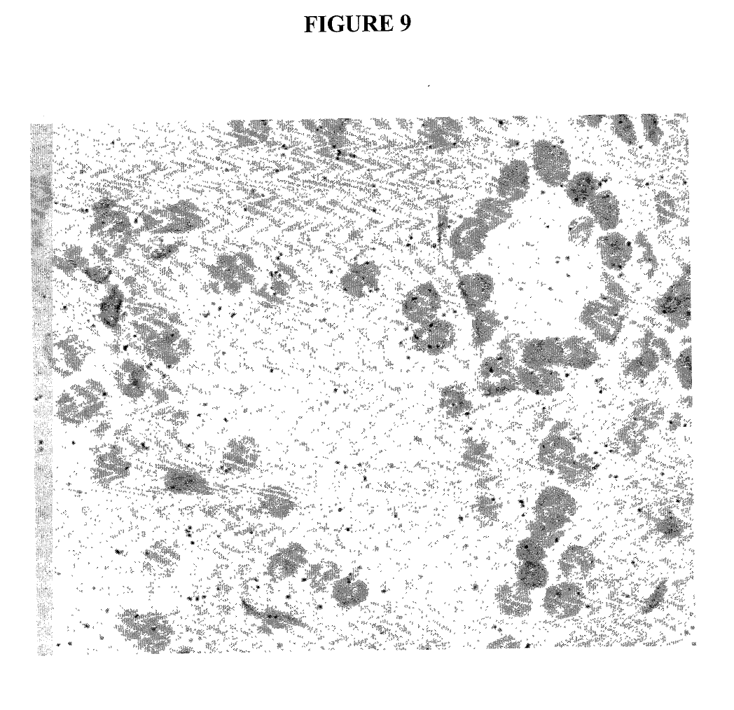

FIGS. 8 and 9 display sections of normal renal artery obtained at nephrectomy from a 24-year-old male with congenital stenosis of the ureteropelvic junction, and show hybridization in a collecting duct epithelium and a renal tubular epithelium, respectively.

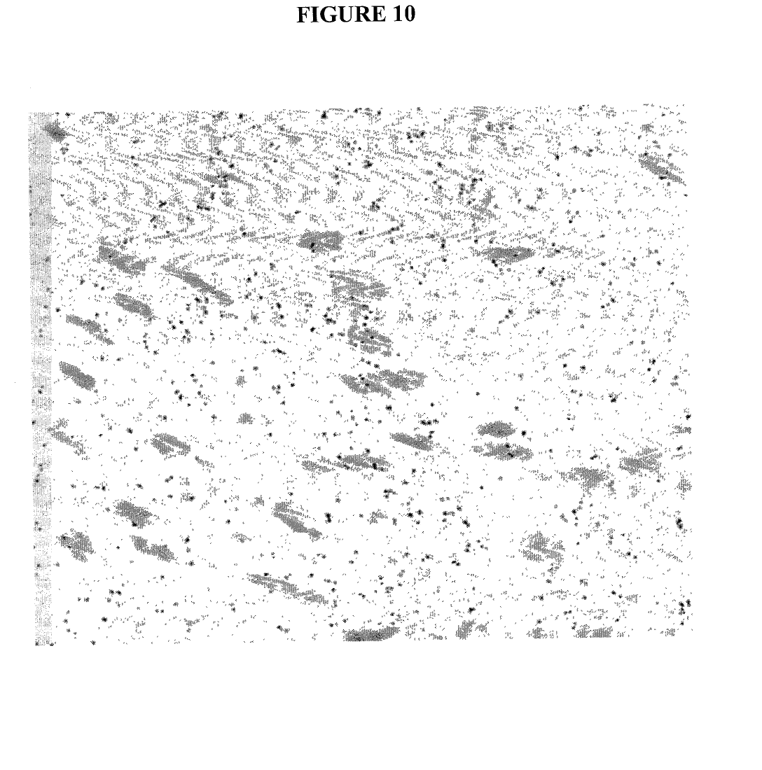

FIG. 10 was a section of a normal heart obtained from a 41-year-old female who died of carcinoma of the cervix, and showed a moderately positive hybridization of the cardiac myocytes.

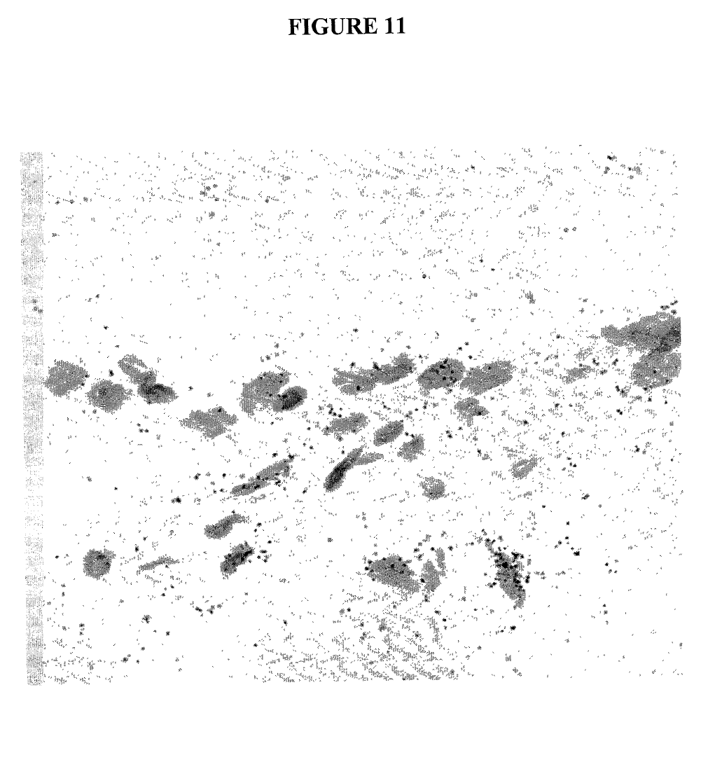

FIG. 11 was a section of a normal heart obtained from a 60-year-old male who died of non-small lung carcinoma, and showed that endothelium was occasionally positive in interstitial vessels.

### Example 11

### Isotopic in Situ Hybridization Study of the ABCA10 Gene

In situ hybridization experiments have been performed using serial arterial, myocardial, and skeletal tissue sections from archival paraffin samples.

Tissue sections were hybridized with radiolabeled cRNA probes of 405 bp corresponding to nucleotides 1383 to 1787 of nucleotide sequence SEQ ID NO: 4, which was then PCR-amplified. The PCR product was then transcribed in vitro with T7 (antisense) and SP6 (sense) RNA polymerases in the presence of ³⁵S-uridine 5′-triphosphate. After transcription, the probes were column-purified and separated by electrophoresis on a 5% polyacrylamide gel to confirm size and purity. Tissue sections were digested, and in situ hybridization were perforned as described in Example 10.

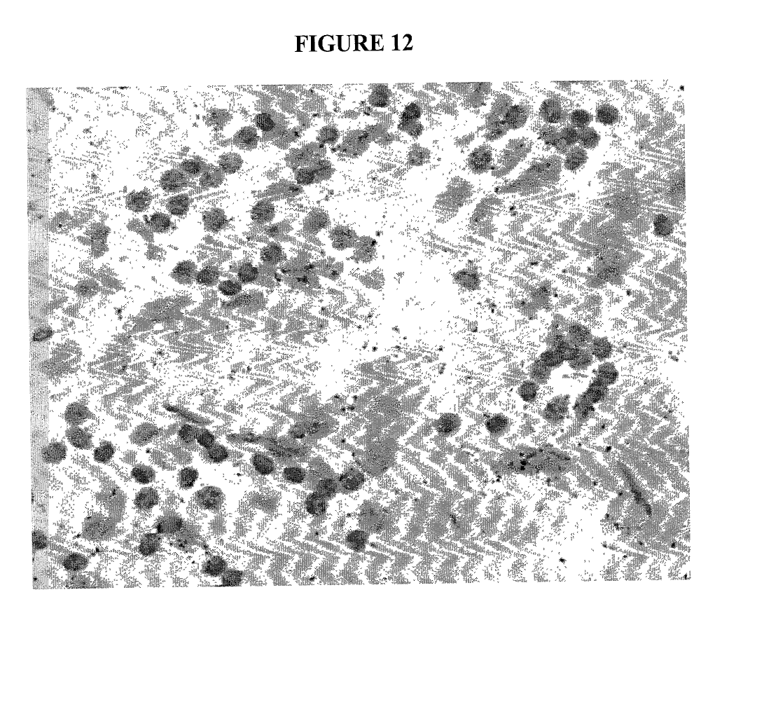

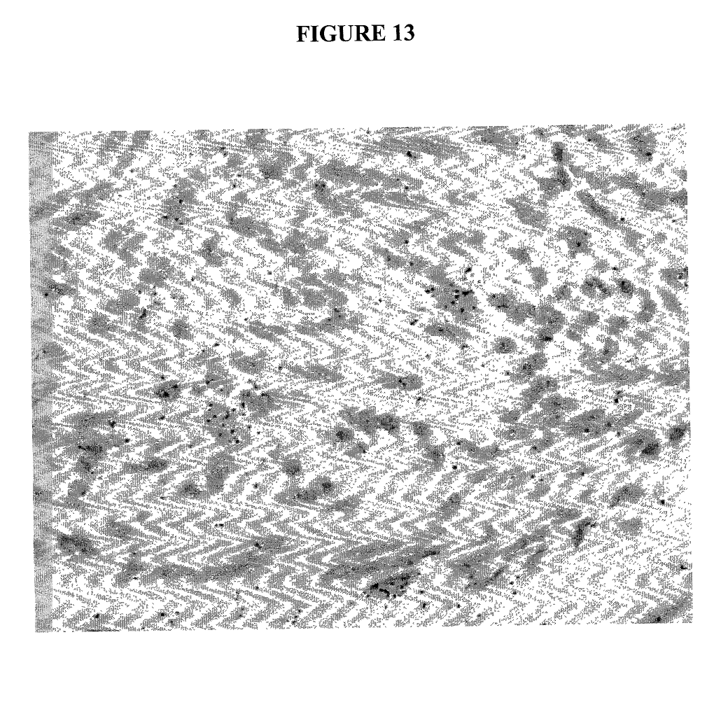

FIGS. 12 and 13 which displays in situ hybridization of arterial tissues showed that the strongest hybridization was identified consistently in macrophages, subsets of lymphocytes, and in Schwann cells of nerves.

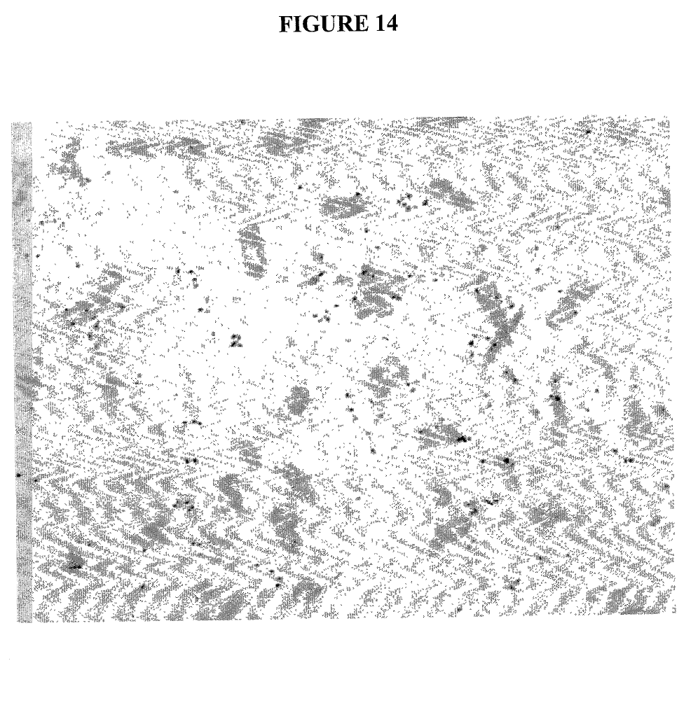

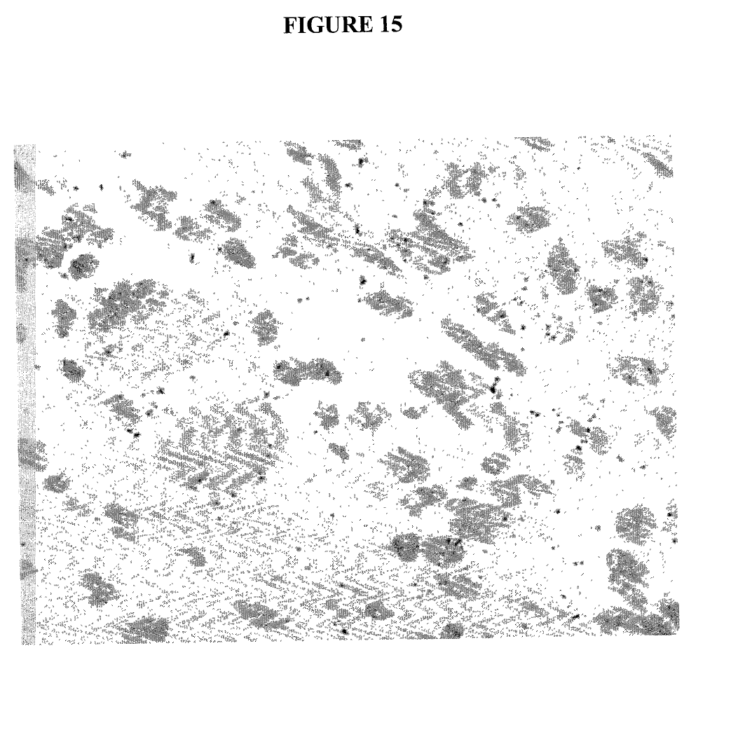

FIG. 14 which display a myocardial tissue section, showed positive signals of macrophages in the atheroma. In an adjacent section of tissue containing a ganglion, subsets of Schwann cells were moderately positive, and ganglion cells showed faint hybridization (FIG. 15).

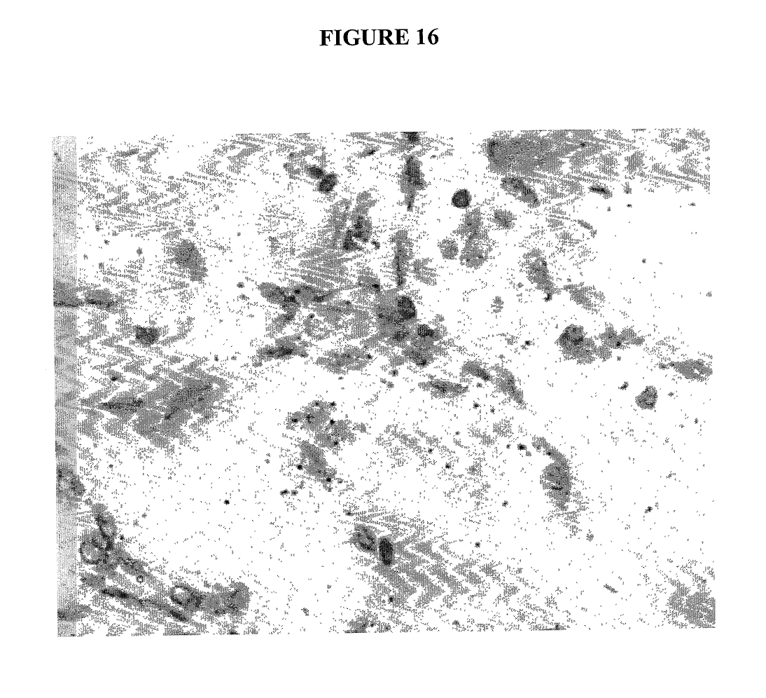

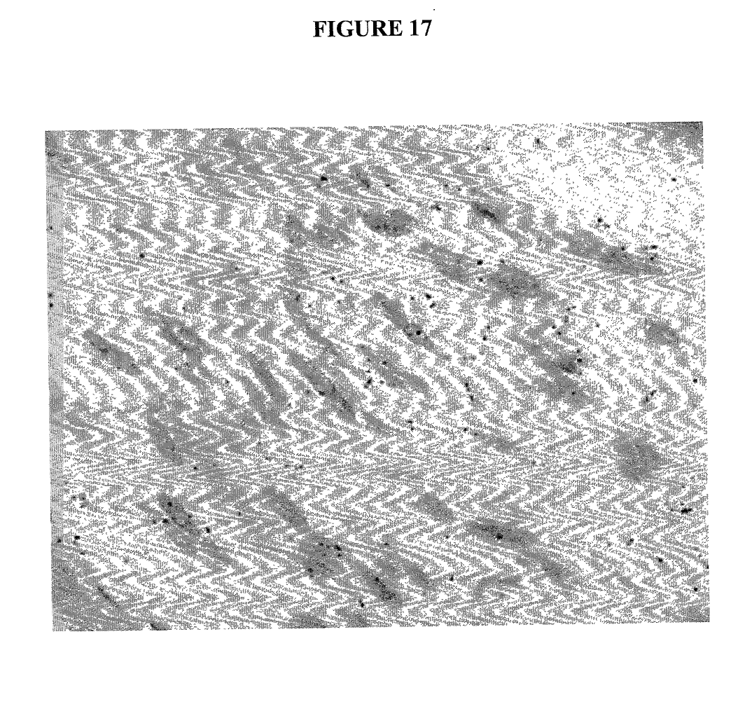

FIG. 16 displays section of skeletal tissue, wherein scattered macrophages were faintly to moderately positive. FIG. 17 showed moderate hybridization of Schwann cells in a nerve.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

We claim:

1. An isolated nucleic acid comprising any one of SEQ ID NOs: 1-4 and 9-126, or of a complementary nucleotide sequence.

2. An isolated nucleic acid comprising at least eight consecutive nucleotides of a nucleotide sequence of any one of SEQ ID NOs: 1-4 and 9-126, or of a complementary nucleotide sequence.

3. An isolated nucleic acid comprising at least 80% nucleotide identity with a nucleic acid comprising any one of SEQ ID NOs: 1-4 and 9-126, or of a complementary nucleotide sequence.

4. The isolated nucleic acid according to claim 3, wherein the nucleic acid has 85%, 90%, 95%, or 98% nucleotide identity with the nucleic acid comprising any one of SEQ ID NOs: 1-4 and 9-126, or of a complementary nucleotide sequence.

5. An isolated nucleic acid that hybridizes under high stringency conditions with a nucleic acid comprising any one of SEQ ID NOs: 1-4 and 9-126 or of a complementary nucleotide sequence.

6. An isolated nucleic acid comprising a nucleotide sequence as depicted in any one of SEQ ID NOs: 1-4 and 9-126 or of a complementary nucleotide sequence.

7. A nucleotide probe or primer specific for any one of ABCA5, ABCA6, ABCA9, and ABCA10 genes, wherein the nucleotide probe or primer comprises at least 15 consecutive nucleotides of a nucleotide sequence of any one of SEQ ID NOs: 1-4 and 9-126 or of a complementary nucleotide sequence.

8. A nucleotide probe or primer specific for an ABCA5 gene, wherein the nucleotide probe or primer comprises a nucleotide sequence of any one of SEQ ID NOS:127-144 or a complementary nucleotide sequence.

9. A nucleotide probe or primer specific for an ABCA6 gene, wherein the nucleotide probe or primer comprises a nucleotide sequence of any one of SEQ ID NOs: 145-172, or of a complementary nucleotide sequence.

10. A nucleotide probe or primer specific for an ABCA9 gene, wherein the nucleotide probe or primer comprises a nucleotide sequence of any one of SEQ ID NOs: 173-203, or of a complementary nucleotide sequence.

11. A nucleotide probe or primer specific for an ABCA10 gene, wherein the nucleotide probe or primer comprises a nucleotide sequence of any one of SEQ ID NOs: 204-217 or of a complementary nucleotide sequence.

12. A method of amplifying a region of the nucleic acid according to claim 1, wherein the method comprises:
a) contacting the nucleic acid with two nucleotide primers, wherein the first nucleotide primer hybridizes at a position 5′ of the region of the nucleic acid, and the second nucleotide primer hybridizes at a position 3′ of the region of the nucleic acid, in the presence of reagents necessary for an amplification reaction; and
b) detecting the amplified nucleic acid region.

13. A method of amplifying a region of the nucleic acid according to claim 12, wherein the two nucleotide primers are selected from the group consisting of
a) a nucleotide primer comprising at least 15 consecutive nucleotides of a nucleotide sequence of any one of SEQ ID NOs: 1-4 and 9-126 or of a complementary nucleotide sequence;
b) a nucleotide primer according to claim 7;
c) a nucleotide primer comprising a nucleotide sequence of any one of SEQ ID NOs: 127-217, or a nucleic acid having a complementary sequence.

14. A kit for amplifying the nucleic acid according to claim 1, wherein the kit comprises:
a) two nucleotide primers whose hybridization position is located respectively 5′ and 3′ of the region of the nucleic acid; and, optionally,
b) reagents necessary for an amplification reaction.

15. The kit according to claim 14, wherein the two nucleotide primers are selected from the group consisting of
a) a nucleotide primer comprising at least 15 consecutive nucleotides of a nucleotide sequence of any one of SEQ ID NOs: 1-4 and 9-126, or of a complementary nucleotide sequence;
b) nucleotide primer according to claim 7;
c) nucleotide primer comprising a nucleotide sequence of any one of SEQ ID NOs: 127-217, or a nucleic acid having a complementary sequence.

16. The nucleotide probe or primer according to claim 7, wherein the nucleotide probe or primer comprises a marker compound.

17. A method of detecting a nucleic acid according to claim 1, wherein the method comprises:
a) contacting the nucleic acid with a nucleotide probe selected from the group consisting of
1) a nucleotide probe comprising at least 15 consecutive nucleotides of a nucleotide sequence of any one of SEQ ID NOs: 1-4 and 9-126, or of a complementary nucleotide sequence;
2) a nucleotide primer according to claim 7;
3) a nucleotide probe comprising a nucleotide sequence of any one of SEQ ID NOs: 127-217, or of a complementary nucleotide sequence; and
b) detecting a complex formed between the nucleic acid and the probe.

18. The method of detection according to claim 17, wherein the probe is immobilized on a support.

19. A kit for detecting the nucleic acid according to claim 1, wherein the kit comprises
a) a nucleotide probe selected from the group consisting of
1) a nucleotide probe comprising at least 15 consecutive nucleotides of a nucleotide sequence of any one of SEQ ID NOs: 1-4 and 9-126, or of a complementary nucleotide sequence;
2) a nucleotide primer according to claim 7; and
3) a nucleotide probe comprising a nucleotide sequence of any one of SEQ ID NOs: 127-217, or of a complementary nucleotide sequence, and, optionally,
b) reagents necessary for a hybridization reaction.

20. The kit according to claim 19, wherein the probe is immobilized on a support.

21. A recombinant vector comprising the nucleic acid according claim 1.

22. The vector according to claim 21, wherein the vector is an adenovirus.

23. A recombinant host cell comprising the recombinant vector according to claim 21.

24. A recombinant host cell comprising the nucleic acid according claim 1.

25. An isolated nucleic acid encoding a polypeptide comprising an amino acid sequence of any one of SEQ ID NOS: 5-8.

26. A recombinant vector comprising the nucleic acid according to claim 25.

27. A recombinant host cell comprising the nucleic acid according to claim 25.

28. A recombinant host cell comprising the recombinant vector according to claim 26.

29. An isolated polypeptide selected from the group consisting of
a) a polypeptide comprising an amino acid sequence of any one of SEQ ID NOs: 5-8;
b) a polypeptide fragment or variant of a polypeptide comprising an amino acid sequence of any one of SEQ ID NOs: 5-8; and
c) a polypeptide homologous to a polypeptide comprising amino acid sequence of any one of SEQ ID NOS: 5-8.

30. An antibody directed against the isolated polypeptide according to claim 29.

31. The antibody according to claim 30, wherein the antibody comprises a detectable compound.

32. A method of detecting a polypeptide, wherein the method comprises
a) contacting the polypeptide with an antibody according to claim 31; and
b) detecting an antigen/antibody complex formed between the polypeptide and the antibody.

33. A diagnostic kit for detecting a polypeptide, wherein the kit comprises
a) the antibody according to claim 31; and
b) a reagent allowing detection of an antigen/antibody complex formed between the polypeptide and the antibody.

34. A composition comprising the nucleic acid according to claim 1 and a physiologically-compatible excipient.

35. A composition comprising the recombinant vector according to claim 21 and a physiologically-compatible excipient.

36. Use of the nucleic acid according to claim 1 for the manufacture of a medicament intended for the prevention and/or treatment of a subject affected by a dysfunction in the reverse transport of cholesterol.

37. Use of a recombinant vector according to claim 21 for the manufacture of a medicament for the prevention and/or treatment of subjects affected by a dysfunction in the lipophilic subtance transport.

38. Use of any one of isolated ABCA5, ABCA6, ABCA9, and ABCA10 polypeptides comprising an amino acid sequence of SEQ ID NOS: 5-8 for the manufacture of a medicament intended for the prevention and/or treatment of subjects affected by a dysfunction in the lipophilic subtance transport.

39. A composition comprising a polypeptide comprising an amino acid sequence of any one of SEQ ID NOs: 5-8, and a physiologically-compatible excipient.

40. Use of any one of isolated ABCA5, ABCA6, ABCA9, and ABCA10 polypeptides comprising an amino acid sequence of any one of SEQ ID NOs: 5-8 for screening an active ingredient for the prevention or treatment of a disease resulting from a dysfunction in the lipophilic subtance transport.

41. Use of a recombinant host cell expressing any one of the ABCA5, ABCA6, ABCA9, and ABCA10 polypeptides comprising an amino acid sequence of SEQ ID NOs: 5-8 for screening an active ingredient for the prevention or treatment of a disease resulting from a dysfunction in the lipophilic subtance transport.

42. A method of screening a compound active on cholesterol metabolism, an agonist, or an antagonist of any one of the ABCA5, ABCA6, ABCA9, and ABCA10 polypeptides, wherein the method comprises
a) preparing a membrane vesicle comprising at least one of the ABCA5, ABCA6, ABCA9, and ABCA10 polypeptides and a lipid substrate comprising a detectable marker;
b) incubating the vesicle obtained in step a) with an agonist or antagonist candidate compound;
c) qualitatively and/or quantitatively measuring a release of the lipid substrate comprising the detectable marker; and
d) comparing the release of the lipid substrate measured in step b) with a measurement of a release of a labeled lipid substrate by a membrane vesicle that has not been previously incubated with the agonist or antagonist candidate compound.

43. A method of screening a compound active on cholesterol metabolism, an agonist, or an antagonist of any one of ABCA5, ABCA6, ABCA9, and ABCA10 polypeptides, wherein the method comprises
a) incubating a cell that expresses at least one of the ABCA5, ABCA6, ABCA9, and ABCA10 polypeptides with an anion labeled with a detectable marker;
b) washing the cell of step a) whereby excess labeled anion that has not penetrated into the cell is removed;
c) incubating the cell obtained in step b) with an agonist or antagonist candidate compound for any one of the ABCA5, ABCA6, ABCA9, and ABCA10 polypeptide;
d) measuring efflux of the labeled anion from the cell; and
e) comparing the efflux of the labeled anion determined in step d) with efflux of a labeled anion measured with a cell that has not been previously incubated with the agonist or antagonist candidate compound.

44. An implant comprising the recombinant host cell according to claim 23.
